(19) United States
(12) Patent Application Publication
(10) Pub. No.: US 2021/0214607 A1
(43) Pub. Date: Jul. 15, 2021

(54) LONG WAVELENGTH EMITTING CHEMILUMINESCENT PROBES

(71) Applicants: NEMIS Technologies AG, Dübendorf (CH); Ramot at Tel-Aviv University Ltd., Tel aviv (IL)

(72) Inventors: Julian IHSSEN, St. Gallen (CH); Urs SPITZ, St. Gallen (CH); Doron SHABAT, Tel-Aviv (IL); Ori GREEN, Tel-Aviv (IL); Nir HANANYA, Tel-Aviv (IL)

(21) Appl. No.: 17/058,453

(22) PCT Filed: May 24, 2019
(86) PCT No.: PCT/EP2019/063419

§ 371 Date: Nov. 24, 2020

(30) Foreign Application Priority Data
May 25, 2018 (EP) 18174403.8
Nov. 13, 2018 (EP) 18205983.2
Apr. 25, 2019 (EP) 19171040.9

(51) Int. Cl. C09K 11/06; C07F 5/04; C07D 405/10; C07D 321/00; G01N 33/58; G01N 21/76

(52) U.S. Cl. CPC C09K 11/06; C07F 5/04; C07D 405/10; C09K 2211/1018; G01N 33/582; G01N 21/763; G01N 21/766; C07D 321/00

(57) ABSTRACT

Improved long wavelength-emitting chemiluminescent probes are easy to synthesize and are well-suited for both in vitro and in vivo applications, but are particularly well-suited for in vivo applications. The wavelengths of the emissions of the probes include those in the orange, red or NIR range. Dioxetane compounds and phenolic ester compounds are included.

### FIELD OF THE INVENTION

The present invention relates to long wavelength emitting probes, in particular to compounds of Formulae Ia, Ib and II, and their applications.

### BACKGROUND OF THE INVENTION

Optical imaging modalities have become powerful tools for noninvasive visualization of biomolecular systems and whole body (e.g. animals or human) in real-time with high spatial resolution. Moreover, imaging systems are relatively inexpensive, easy to use, portable, and adaptable to acquire physiological and functional information from microscopic to macroscopic levels.

There are several approaches in optical imaging, among them fluorescence is the most familiar. This technique is widely used for imaging and monitoring various biological processes in-vivo. However, in fluorescence techniques complications arises from auto-fluorescence and light interferences, which typically increases the background noise. One way to overcome this obstacle is by using bioluminescence techniques, which minimize light interference since light is produced from within the body without the use of external light sources.

Currently, bioluminescence techniques rely heavily on transgenic cells that express the enzyme luciferase. For example, in vivo bioluminescence imaging very often requires the use of luciferase-generating transgenic mice, which are then injected with luciferin, which limits the applicability of in vivo bioluminescence imaging techniques.

Chemiluminescence offers significant advantages over fluorescence and bioluminescence techniques since light is generated by a specific chemical reaction that initiates light emission without further enzymatic dependency. Chemiluminescnece has until very recently never been used for imaging in live animals. The examples known are based on Shabat dioxetanes.

Schaap's adamantylidene 1,2-dioxetane probes (Scheme 1, structure I) are the only known compounds that do not require an oxidation step, since the energetic peroxide ring is thermally stable. This grants them a modular activating mechanism. As depicted in Scheme 1, Schaap's adamantylidene-dioxetane based chemiluminescence probe (structure I) is equipped with an analyte-responsive protecting group used to mask the phenol moiety of the probe. Removal of the protecting group by the analyte of interest generates an unstable phenolate-dioxetane species II, which decomposes through a chemiexcitation process to produce the excited intermediate benzoate ester III and adamantanone. The excited intermediate decays to its ground-state (benzoate ester IV) through emission of a blue light photon.

Unfortunately, the chemiluminescent signal generated by Schaap's systems is not efficient under physiological conditions, and the blue photons released by these systems tend to be absorbed by organic tissues, in particular blood. The emission spectrum of a suitable substrate for live animal imaging must not fully overlap with the absorption spectrum of hemoglobin. Hence, in order to make Schaaps' dioxetane relevant to full body imaging, an increase of the light wavelength toward the long wavelength (in particular red/NIR) region is desired. Up until recently, in-vitro and in-vivo imaging assays could not be applied without the use of a surfactant or complex supramolecular systems. The limitation of Schaap dioxetanes arises for the very low quantum yield in hydrophilic environments. For this reason special sensitizers (typically of polymeric nature) are needed in all assays based on Shaap dioxetanes in order to get a useful signal. The need for such sensitizers severely limits the potential uses of substrates for imaging purposes since substrates are unlike to diffuse at similar rates in biological matrices especially if such senzitizers are made from large molecules such as polymers.

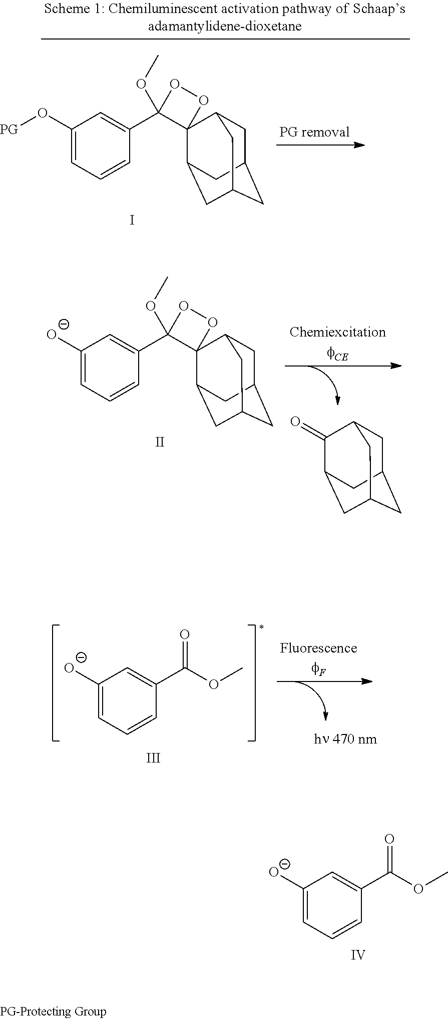

The chemiluminescent activation pathway of compounds of Formula Ia corresponds to the one shown in Scheme 1.

The compound of Formula Ib, which is a singlet oxygen sensitive prove, first reacts with singlet oxygen to form the dioxetane unit followed by the chemiluminescent activation pathway shown in Scheme 1.

International Publication No. WO2017/130191 discloses chemiluminescence probes based on the Schaap's adamantylidene-dioxetane probe, wherein chemiluminescence emission is amplified through a direct mode of action, more particularly wherein the Schaap's adamantylidene-dioxetane probe is substituted at the ortho position of the phenolic ring with a π* acceptor group such as an acrylate and acrylonitrile electron-withdrawing group so as to increase the emissive nature of the benzoate species (Scheme 2). As shown in this publication, luminophores as disclosed allow for the enzymatic hydrolysis and the chemiexcitation process to occur concurrently under physiological conditions, with remarkable chemiluminescence intensities. Those luminophores are extremely bright in aqueous solutions. However, the light that is emitted by them is green (about 530 nm), which is absorbed by tissue and thus, might cause difficulties when engaging whole body imaging.

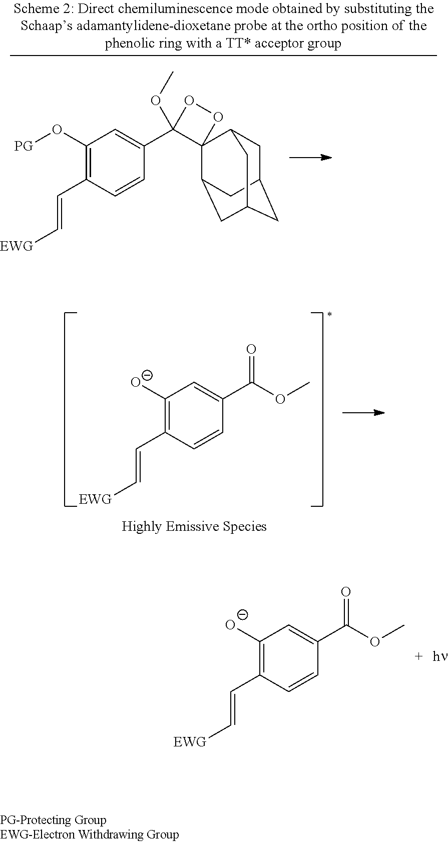

Therefore, NIR-emitting dioxetane probes have recently been developed and reported in international publication no. WO 2018/216013. These probes are based on 4-(dicyanomethylene)-4H-chromen-2-yl and 5,5-dimethyl-3-cyano-2-dicyanomethylene-2,5-dihydrofuran-4-yl substituents acting as π-acceptors and shifting the emission to long wavelengths, which, however renders their synthesis rather complex and cumbersome. Additionally, these substituents are rather hydrophobic such that these probes tend to suffer from solubility issues in aqueous media. Therefore, if used for in vitro or in vivo imaging, these probes further have to be provided with a solubility-enhancing substituent (e.g., an acrylic acid substituent), which, however, renders their synthesis even more complex.

### OBJECT OF THE INVENTION

Thus, it is an object of the present invention to provide improved long wavelength-emitting (in particular emission in the orange, red or NIR range) chemiluminescent probes that are easy to synthesize. In particular, it is an object of the present invention to provide long wavelength-emitting chemiluminescence probes that are easy to synthesize and that are well suitable for in vitro and in vivo applications, in particular for in vivo applications.

### SUMMARY OF THE INVENTION

The above object is achieved by compounds of Formula Ia and Ib defined in claim 1 of the present application. As set out in more detail below, it was surprisingly found that the compounds of Formula Ia and Ib show long wavelength emission (in particular an emission maximum at about 590 nm or more), are easy to synthesize and show good solubility in aqueous media.

In a first aspect, the present invention provides a compound of Formula Ia or Ib as generally defined in claim 1.

In a second aspect, the present invention provides a compound of Formula II as defined in claim 7.

In a third aspect, the present invention provides a composition comprising a compound of Formula Ia or Ib and a carrier.

In a fourth aspect, the present invention provides a ready-for-use injectable solution comprising a compound of Formula Ia or Ib.

In a fifth aspect, the present invention provides a compound of Formula Ia or Ib, a composition comprising a compound of Formula Ia or Ib and a carrier, or a ready-for-use injectable solution comprising a compound of Formula Ia or Ib for use in in vivo diagnostics or imaging.

In a sixth aspect, the present invention provides the use of a compound of Formula Ia or Ib for in vitro imaging.

In a seventh aspect, the present invention provides the use of a compound of Formula Ib in an in vitro assay for the detection of singlet oxygen.

In an eights aspect, the present invention provides the use of a compound of Formula Ia in any in vitro assay for the detection of a peroxide, reactive oxygen species, reactive nitrogen species, or of an enzyme.

In a ninth aspect, the present invention provides a method for determining the presence, or measuring the level, of an analyte in a sample.

In a tenth aspect, the present invention provides the use of a compound of Formula Ia or Ib as a label for a biomolecule.

In an elevenths aspect, the present invention provides a biomolecule, characterized in that it is bound to a compound of Formula Ia or Ib as a label.

In a twelvths aspect, the present invention provides a biomolecule of the elevenths aspect for use in diagnosis.

### DESCRIPTION OF THE FIGURES

FIG. 1 shows the chemiluminescent kinetic profile of compound Ia1.

FIG. 2 shows the total light emission with or without the presence of H₂O₂ of compound Ia1.

FIG. 3 shows the chemiluminescent response to various H₂O₂ concentrations of compound Ia1.

FIG. 4 shows the chemiluminescent emission spectrum of compound Ia2.

FIG. 5 shows the chemiluminescent kinetic profile of compound Ia3.

FIG. 6 shows the total light emission with or without the presence of H₂O₂ of compound Ia3.

FIG. 7 shows a comparison of the chemiluminescent kinetic profiles of compounds Ia1 and Ia3.

FIG. 8 shows the chemiluminescence kinetic profile (FIG. 8A) and the total light emission (FIG. 8B) of compounds SAG 2-173 and OG 5-160

FIG. 9 shows the chemiluminescent properties of compound CLHP-555.

FIG. 10 shows the chemiluminescent properties of compound CLHP-595.

### DETAILED DESCRIPTION OF THE INVENTION

Currently, bioluminescent imaging methods are restricted by the required expression of a luciferase enzyme. Hence, animals must be transgenic or suitable cells must be implanted, which however has a number of rather severe drawbacks. In contrast, chemiluminescence based methods disclosed herein may rely on the intrinsic biochemical profile of cells such as the over-expression of certain enzymes such as cathepsines or caspases or the elevated levels of metabolites species such as hydrogen peroxide or singlet oxygen in target cells. Further, other more robust reporter gene systems such as LacZ (expressing beta-D-galactosidaase) or GUS (expressing beta-D-glucuronidase) instead of the rather tedious luciferin/luciferase system may be used. Although long wavelength-emitting dioxetane probes have recently been developed, there is still room for improvement, in particular from a synthesis and solubility point of view. In this respect, the inventors of the present invention have surprisingly found that dioxetane compounds of Formulae Ia and Ib are highly efficient probes for such methods. In particular, it has been found that dioxetane compounds of Formulae Ia and Ib are highly efficient probes for in vivo and in vitro bioluminescence imaging. In particular, it has been found that compounds of Formulae Ia and Ib show long wavelength emission (in particular emission in the orange, red or NIR range), are easy to synthesize and show good solubility in aqueous media. Further, dioxetane compounds of Formulae Ia and Ib function without any auxiliary chemicals and can be triggered by a wide range of biochemical or chemical events or conditions. Chemiluminescence imaging systems must be single component in order to be applicable for imaging purposes, particularly in live animals. All of these properties make compounds of Formulae Ia and Ib particularly suitable for in vivo and in vitro bioluminescence imaging.

In a first aspect, the present invention relates to a compound of Formula Ia or Ib

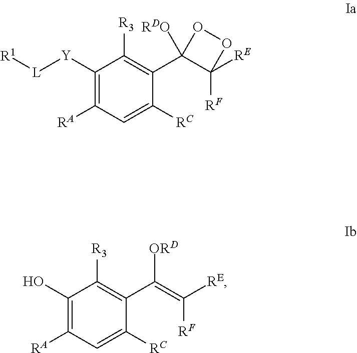

wherein the substituents are defined as follows:
R^{D} is selected from a linear or branched C1-C18 alkyl or C3-C7 cycloalkyl. Preferably, R^{D} is methyl or ethyl. More preferably, R^{D} is methyl.
R^{E} and R^{F} are independently selected from a branched C3-C18 alkyl or C3-C7 cycloalkyl, or R^{E} and R^{F} together with the carbon atom to which they are attached form an optionally substituted fused, spiro or bridged cyclic or polycyclic ring. Preferably, R^{E} and R^{F} together with the carbon atom to which they are attached form adamantyl, which may be substituted.
R³ is —H, —F, —Cl, —Br, —I, —CF₃, —NO₂, —CN, —COOR^{XX}, —C(O)R^{XX}, —SO₂R^{XX} or R². Preferably, R³ is Cl.
R^{A} and R^{C} are independently selected from —H, —F, —Cl, —Br, —I, —CF₃, —NO₂, —CN, —R^{x}COOR^{XX}, —COOR^{XX}, —C(O)R^{XX}, —SO₂R^{XX} and R².
R^{x} is linear or branched C1-C6 alkylene or linear or branched C1-C6 alkenylene, preferably —CH═CH—.
R^{XX} is linear or branched C1-18 alkyl, C2 to C8 alkenyl or C2 to C8 alkynyl chain, or —H.
At least one, preferably one, of R³, R^{A} and R^{C} is R². Preferably, R³ is as defined above and R^{A} is R² and R^{C} is H, or R³ is as defined above and R^{A} is H and R^{C} is R².
R² is selected from the group consisting of

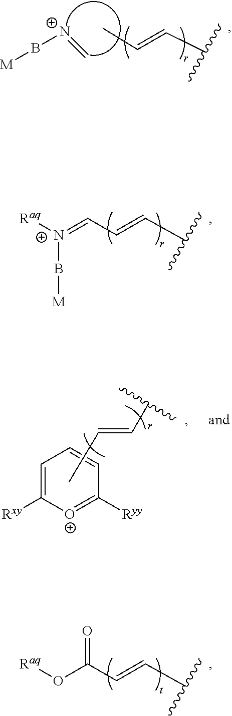

wherein

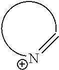

denotes a mono- or polycyclic, aromatic or nonaromatic ring system comprising the moiety

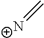

as a ring member,
wherein the moiety

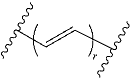

is connected to

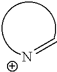

via an atom, which is a member of said mono- or polycyclic, aromatic or nonaromatic ring system, provided that a delocalized Tr-system extends from the positively charged nitrogen atom of

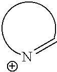

via moiety

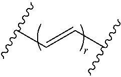

to the central aromatic ring of the compound of Formula Ia or Ib.

Each ring of said mono- or polycyclic, aromatic or nonaromatic ring system may be substituted with one or more groups selected from —OH, —CN, —SO₃⁻, linear or branched C1-C6 alkyl, linear or branched C2-C6 alkenyl, linear or branched C2-C6 alkynyl, a polyethylene glycol chain or a polypropylene glycol chain.

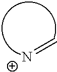

may be substituted with one or two negatively charged substituent(s) in ortho position to the positively charged nitrogen atom. Said negatively charged substituents are preferably selected from −COO⁻ and —SO₃⁻.
r is selected from the group consisting of 1, 2, 3, 4, 5, and 6. Preferably, r is 1.
R^{xy} and R^{yy} are independently selected from —H, linear or branched C1-C6 alkyl, linear or branched C2-C6 alkenyl, linear or branched C2-C6 alkynyl, and C3-C7 cycloalkyl groups. Preferably R^{xy} and R^{yy} are independently selected from methyl, ethyl, propyl, isopropyl, butyl, sec-butyl, and tert-butyl.
R^{aq} is a linear or branched C1 to C8 alkyl (preferably C2 to C6 alkyl), a linear or branched C2 to C8 alkenyl, a linear or branched C2 to C8 alkynyl, or a linear or branched C4 to C12 heteroalkyl, wherein the linear or branched C1 to C8 alkyl, the linear or branched C2 to C8 alkenyl, the linear or branched C2 to C8 alkynyl, or the linear or branched C4 to C12 heteroalkyl may be substituted with one or more groups selected from —OH, —COOH, halogen, preferably —Cl or —F, and —NH₂ and wherein the linear or branched C1 to C8 alkyl, the linear or branched C2 to C8 alkenyl or the linear or branched C2 to C8 alkynyl chain may comprise one or more —O— or —CO— groups within the chain.
M is an optionally present group, wherein
(i), if M is absent, B is —O^{⊖}, H, a linear or branched C1 to C8 alkyl, preferably a linear or branched C2 to C6 alkyl, a linear or branched C2 to C8 alkenyl or a linear or branched C2 to C8 alkynyl chain,
wherein the linear or branched C1 to C8 alkyl, C2 to C8 alkenyl or C2 to C8 alkynyl chain may be substituted with one or more groups selected from —OH, —COOH, halogen, preferably —Cl or —F, —NH₂ and a group capable of binding to a functional group of a peptide, endolysine, or protein, wherein said functional group of a peptide, endolysine, or protein is selected from an amino, carboxy, or mercapto group, thus allowing for binding said peptide, endolysine, or protein to B; and
wherein the linear or branched C1 to C8 alkyl, C2 to C8 alkenyl or C2 to C8 alkynyl chain may comprise one or more —O— or —CO— groups within the chain,
   l preferably B is —O^{⊖}, H, —CH₃, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —CH═CH₂, —CH═CHCH₃, —CH₂CH═CH₃, or a linear or branched C4-C8 alkenyl group,
   preferably, if M is absent and B is H,

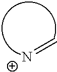

is substituted with one or two, preferably two, —COO⁻ groups in ortho position to the positively charged nitrogen atom, or
(ii) if M is present, B is a linear or branched C1 to C8 alkylene, preferably C2 to C6 alkylene, a linear or branched C2 to C8 alkenylene or linear or branched C2 to C8 alkynylene chain,
wherein the linear or branched C1 to C8 alkylene, C2 to C8 alkenylene or C2 to C8 alkynylene chain may be substituted with one or more groups selected from —OH, —COOH, halogen, preferably —Cl or —F, —NH₂ and a group capable of binding to a functional group of a peptide, endolysine, or protein, wherein said functional group of a peptide, endolysine, or protein is selected from an amino, carboxy, or mercapto group, thus allowing for binding said peptide, endolysine, or protein to B; and
wherein the linear or branched C1 to C8 alkylene, C2 to C8 alkenylene or C2 to C8 alkynylene chain may comprise one or more —O— or —CO— groups within the chain,
   preferably B is —CH₂—, —(CH₂)₂—, —(CH₂)₃—, —(CH₂)₄—, —(CH₂)₅—, —(CH₂)₆—, —(CH₂)₇—, —(CH₂)₈—, —CH═CH—, —CH₂CH═CHCH₂—, a linear or branched C6 alkenylene group with one or two double bonds or a linear or branched C8 alkenylene group with one, two or three double bonds.
   M is selected from the group consisting of cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, tetrazole, optionally substituted aryl, optionally substituted alkenyl,

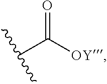

carbonyl having the structure

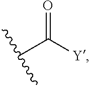

amide, an amide having the structure

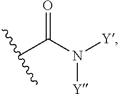

or M is a moiety including one or more groups selected from cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, and tetrazole, optionally substituted aryl, optionally substituted alkenyl,

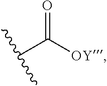

carbonyl having the structure

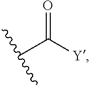

amide, an amide having the structure

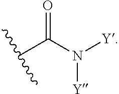

Y″ is —H, an optionally substituted C1-C8 alkyl, optionally substituted C2-C8 alkenyl, optionally substituted C2-C8 alkynyl, an alkali metal ion or a negative charge.
Y′ and Y″ are independently selected from —H, an optionally substituted C1-C8 alkyl, optionally substituted C2-C8 alkenyl, or an optionally substituted C2-C8 alkynyl, or Y′ and Y″ together with the nitrogen atom to which they are attached form an optionally substituted heterocyclic structure, preferably an optionally substituted maleimide group.
Preferably, M is —COOH, —SO₃⁻, a moiety derived from an amino acid, a moiety derived from a monosaccharide or a disaccharide, a moiety derived from a polycarboxylic acid, a moiety derived from polyethylene glycol or polypropylene glycol, or a moiety derived from a polyol. More preferably, M is —COOH or —SO₃⁻.
t is 2, 3, or 4.
R^{aa} is —H, a linear or branched C1-6 alkyl (preferably ethyl or methyl, more preferably methyl), a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group such as

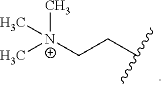 [SMILES: *CC[N+](C)(C)C]

Y is absent or is —O—, provided that Y is absent if R¹ is —B(Z)(Z′) or —B(Z″)3⁻Kat⁺ and L is absent.
Z and Z′ are independently selected from R^{ab} and OR^{ac}, wherein
R^{ab} is selected from the group consisting of —OH, —O⁻Kat⁺, optionally substituted C1-C4 alkyl, optionally substituted C2-C4 heteroalkyl, optionally substituted C2-C4 alkenyl, optionally substituted C2-C4 heteroalkenyl, optionally substituted C2-C4 alkynyl, optionally substituted C2-C4 heteroalkynyl, optionally substituted C5-C6 aryl, optionally substituted C5-C6 heteroaryl, optionally substituted C6-C10 aralykl, and optionally substituted C6-C10 heteroaralkyl, and
R^{ac} is selected from the group consisting of —H, optionally substituted C1-C4 alkyl, optionally substituted C2-C4 heteroalkyl, optionally substituted C2-C4 alkenyl, optionally substituted C2-C4 heteroalkenyl, optionally substituted C2-C4 alkynyl, optionally substituted C2-C4 heteroalkynyl, optionally substituted C5-C6 aryl, optionally substituted C5-C6 heteroaryl, optionally substituted C6-C10 aralykl, and optionally substituted C6-C10 heteroaralkyl, or wherein two R^{ab}, two R^{ac} or one R^{ab} and one R^{ac} together with their intervening atoms form a 5- to 7-membered optionally substituted heterocyclic ring, preferably a saturated optionally substituted heterocyclic ring.
Z″ is selected from —F, —Cl, —Br, and —I. Preferably, Z″ is —F.
Kat⁺ is an organic or inorganic cation. Preferably, Kat⁺ is an alkali metal cation.
L is absent or is a linker selected from the group consisting of moieties L1 to L8

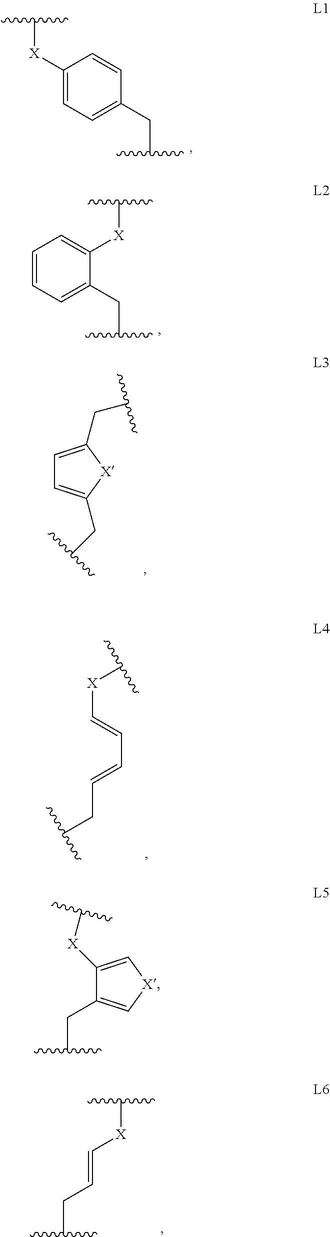

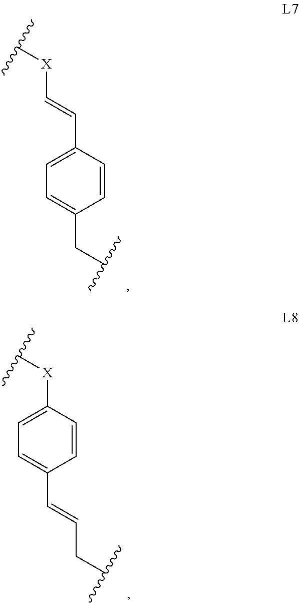

X is absent or is —O—, —NH—, —NR^{G}—, —S—, or —NH—COO— wherein the COO-moiety is bound to R¹, wherein R^{G} is selected from a substituted or unsubstituted C1-C12 alkyl. Preferably, X is absent or is —O— or —NH—.
X is absent if R¹ is —B(Z)(Z′), —B(Z″)₃⁻Kat⁺, —NO₂ or an azide group.
X′ is selected from —S—, —O—, —NH—, and —NR^{G}—, wherein R^{G} is selected from a substituted or unsubstituted C1-C12 alkyl.
X is connected to R¹.
Each of L1 to L8 is optionally functionalized with a group capable of binding to a functional group of a peptide, endolysine, or protein, or a cell membrane-permeable group, wherein said functional group of a peptide, endolysine, or protein is selected from an amino, carboxy, or mercapto group, thus allowing for binding said peptide, endolysine, or protein to L.
L is absent and R¹ is —B(Z)(Z′), —B(Z″)₃⁻Kat⁺ if Y is absent.
Y is —O— if L is present.
R¹ is an analyte-responsive group capable of reacting with an analyte, wherein if L is present and X is present, then X—R¹ is converted into a XH group upon reaction of R¹ with said analyte, or
if L is present and X is absent, then R¹ is converted into a π-donor group upon reaction of R¹ with said analyte, or
if L and Y are absent and R¹ is —B(Z)(Z′) or —B(Z″)3⁻Kat⁺, then R¹ is converted into a —OH group upon reaction of R¹ with said analyte, or
if L is absent and Y is —O—, then the —O—R¹ moiety is converted into a —OH group upon reaction of R¹ with said analyte.

The term “long wavelength range”, or the like, as used herein, refers to a wavelength of at least 550 nm, preferably at least 580 nm, more preferably at least 590 nm, in particular a range covering orange light (i.e. light having a wavelength of about 590 nm to about 625 nm), red light (i.e. light having a wavelength of about 625 nm to about 740 nm) and the NIR range.

The term “alkyl”, as used herein, refers to a linear or branched hydrocarbon radical and includes, for example, methyl, ethyl, n-propyl, isopropyl, n-butyl, sec-butyl, tert-butyl, n-pentyl and so on. In other words, an alkyl substituent is an alkane missing one hydrogen. For example, the term “C₁-C₁₂ alkyl” (or “C1-C12 alkyl” or the like), as used herein, refers to an “alkyl” having 1 to 12 carbon atoms. The “alkyl” may be substituted or unsubstituted.

The term “cycloalkyl”, as used herein, refers to a cyclic alkyl.

The term “alkenyl”, as used herein, refers to a linear or branched hydrocarbon radical having one or more carbon-carbon double bonds. The “alkenyl” may be substituted or unsubstituted.

The term “alkynyl”, as used herein, refers to a linear or branched hydrocarbon radical having one or more carbon-carbon triple bonds. The “alkynyl” may be substituted or unsubstituted.

The terms “heteroalkyl”, “heteroalkenyl”, and “heteroalkynyl”, as used herein, refer to the corresponding hydrocarbyl (alkyl, alkenyl, and alkynyl) group, which contains one or more O, S or N heteroatoms or combinations thereof within the backbone residue; thus, at least one carbon atom of a corresponding alkyl, alkenyl, or alkynyl group is replaced by one of the specified heteroatoms to form a heteroalkyl, heteroalkenyl, or heteroalkynyl group. The “heteroalkyl”, “heteroalkenyl” and “heteroalkynyl” may be substituted or unsubstituted.

The term “aryl”, as used herein, refers to an aromatic group consisting of a single ring or condensed multiple rings such as, but not limited to, phenyl, naphthyl, phenanthryl, and biphenyl. The “aryl” may be substituted or unsubstituted.

The term “heteroaryl”, as used herein, refers to an aromatic group containing at least one heteroatom (i.e. an atom different from carbon or hydrogen, e.g. N, S, O, P, Se, Te, preferably N, S, O, P) as a ring member. The “heteroaryl” may be substituted or unsubstituted.

The term, “aromatic group”, “aromatic moiety”, “aromatic ring system” or the like, as used herein, includes both aromatic hydrocarbon groups and heteroaromatic groups (i.e. aromatic groups containing a heteroatom (preferably, S, O, N, Te, Se, more preferably S, O or N) as ring member). Preferably, an aromatic group, aromatic moiety, aryl or the like, as referred to herein, is an aromatic hydrocarbon group.

The term “alkylene”, as used herein, refers to a bifunctional saturated linear or branched hydrocarbon chain and includes, for example, methylene (—CH₂—), ethylene (—CH₂—CH₂—), propylene (—CH₂—CH₂—CH₂—), 2-methylpropylene [—CH₂—CH(CH₃)—CH₂—], hexylene [—(CH₂)₆-] and the like. The “alkylene” may be substituted or unsubstituted.

The term “alkenylene”, as used herein, refers to a bifunctional linear or branched hydrocarbon chain including at least one carbon-carbon double bond, for example ethenylene (—CH═CH—), —CH₂—CH═CH—, and the like. The “alkenylene” may be substituted or unsubstituted.

The term “alkynylene”, as used herein, refers to a bifunctional linear or branched hydrocarbon chain including at least one carbon-carbon triple bond. The “alkynylene” may be substituted or unsubstituted.

Suitable substituents of an “optionally substituted” or “substituted” group are independently

(1) halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}—; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o}₂; or —(C₁₋₄ straight or branched)alkylene)C(O)O—N(R^{o})₂,
wherein each R^{o} is independently hydrogen, C₁₋₆ alkyl, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur; or
(2) ═O, ═S, ═NNR^{o}₂, ═NNHC(O)R^{o}, ═NNHC(O)OR^{o}, ═NNHS(O)₂R^{o}, ═NR^{o}, =NOR^{o}, —O(C(R^{o}₂))₂₋₃O—, or —S(C(R^{o}₂))₂₋₃S—, wherein each independent occurrence of R^{o} is selected from hydrogen, C₁₋₆ alkyl, or an unsubstituted 5-6-membered saturated, partially unsaturated, or aryl ring having 0-4 heteroatoms independently selected from nitrogen, oxygen, or sulfur.

The symbol “ [symbol] ” terminating a bond of a chemical moiety, as used herein, indicates the connection to another moiety. For example, a compound of Formula Ia, wherein R^{A} is

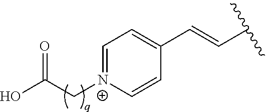

is a compound of the following structure:

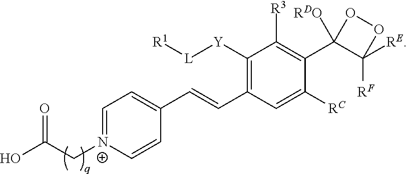

The term “moiety derived from an amino acid”, as used herein, refers to a moiety formed from an amino acid by binding said amino acid to another moiety (e.g., group B), e.g. by means of standard coupling reactions. For example, the amino acid may be bound by coupling its carboxylic acid group to an amine group or by coupling its amine group to a carboxylic acid group or by coupling its hydroxyl group, if present, to a carboxylic acid group.

The amino acid is preferably selected from arginine, histidine, lysine, aspartic acid, glutamic acid, serine, threonine, asparagine, glutamine, cysteine, glycine and proline. More preferably, the amino acid is selected from arginine, histidine, lysine, aspartic acid, and glutamic acid. These amino acids are present in a charged form under physiological conditions which leads to a particularly good solubility of the compound of Formula Ia or Ib in aqueous media. Even more preferably, the amino acid is aspartic acid.

The term “moiety derived from a monosaccharide or a disaccharide”, as used herein, refers to a moiety formed from a monosaccharide or a disaccharide by binding said monosaccharide or a disaccharide to another moiety (e.g., group B), e.g. by means of standard coupling reactions. For example, the monosaccharide may be bound by coupling its hydroxyl group to a carboxylic acid group. One or more hydroxyl groups of the monosaccharide or disaccharide may also be transferred into an amine group or coupled to an amine-comprising moiety thereby indirectly replacing the hydroxyl group by an amine group first, which is then coupled to a carboxylic acid group by means of standard coupling reactions. Alternatively, one or more hydroxyl groups may be oxidized into an aldehyde or a carboxylic acid group first, which is then coupled to an amine group by means of standard coupling reactions.

Preferably, the monosaccharide is selected from the group consisting of glucose, galactose, fructose, xylose, more preferably glucose. Preferably, the disaccharide is selected from the group consisting of sucrose, lactose, maltose, and trehalose.

The term “moiety derived from a polycarboxylic acid”, as used herein, refers to a moiety formed from a polycarboxylic acid by binding said polycarboxylic acid to another moiety (e.g., group B), e.g. by means of standard coupling reactions. For example, a carboxylic acid group of the polycarboxylic acid is coupled to a hydroxyl group by means of standard coupling reactions. The term “polycarboxylic acid”, as used herein, refers to a molecule, which comprises two or more, preferably three or more, carboxylic acid groups, which preferably does not contain atoms other than carbon, hydrogen, oxygen, sulfur, nitrogen, and phosphorous, and which has a ratio of the number of carboxylic acid groups to the total number of carbon atoms of more than 0.1, preferably more than 0.2, more preferably more than 0.3. It has been found that such moieties, due to the high amount of carboxylic acid groups with respect to the total number of carbon atoms, lead to a good solubility in aqueous media.

Polycarboxylic acids that are preferably used in the present invention are malic acid, 1,2,3,4-butanetetracarboxylic acid, citric acid, isocitric acid, succinic acid, methylsuccinic acid, itaconic acid, mesaconic acid, citraconic acid, tartaric acid, aconitic acid, propane-1,2,3-tricarboxylic acid, oxalic acid, malonic acid, glutaric acid, adipic acid, pimelic acid, suberic acid, azelaic acid, sebacic acid, maleic acid, fumaric acid, glutaconic acid, tartronic acid, mesoxalic acid, oxaloacetic acid, aspartic acid, α-hydroxy glutaric acid, arabinaric acid, acetonedicarboxylic acid, α-ketoglutaric acid, glutamic acid, diaminopimelic acid, saccharic acid, EDTA, nitrilotriacetic acid, EGTA, and ethylenediamine-N,N′-disuccinic acid (EDDS). Preferred polycarboxylic acids are polycarboxylic acids comprising 3 to 8 carbon atoms.

The term “moiety derived from polyethylene glycol or polypropylene glycol”, as used herein, refers to a moiety formed from polyethylene glycol or polypropylene glycol by binding said polyethylene glycol or polypropylene glycol molecule to another moiety (e.g. group B), e.g. by means of standard coupling reactions. For example, the terminal hydroxyl group of polyethylene glycol or polypropylene glycol may be coupled to a carboxylic acid group by means of standard coupling reactions.

The term “moiety derived from a polyol”, as used herein, refers to a moiety formed from a polyol by binding said polyol, preferably via one of its —OH groups, to another moiety (e.g., group B), e.g. by means of standard coupling reactions. The term “polyol” as used herein, refers to a compound containing more than one —OH groups.

Polyols that are preferably used in the present invention are selected from sugar alcohols such as ethylene glycol, glycerol, erythritol, threitol, arabitol, xylitol, ribitol, mannitol, sorbitol, galactitol, fucitol, iditol, inositol, volemitol, isomalt, maltitol, lactitol; pentaerythritol, 1,3-propanediol, 1,2,4-butanetriol, 1,2,3-butanetriol, and 1,1,1-Tris(hydroxymethyl)ethane,

Groups capable of binding to a functional group of a peptide, endolysine, or protein are known to the skilled person. Preferably, groups capable of binding to an amino functional group are selected from the group consisting of an aldehyde group; a dialdehyde group having the formula

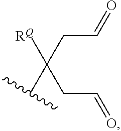

wherein R^{Q} is hydrogen or a 01-C6 alkyl, such as methyl; a carboxylic acid; an acid chloride; and a carboxylic acid NHS ester. Groups capable of binding to a carboxy functional group are preferably selected from the group consisting of an amino group, an alcohol and an acid chloride. Groups capable of binding to a mercapto functional group are preferably selected from the group consisting of a maleimide group.

The term “cell membrane-permeable group”, “cell-permeable group” or the like, as used herein, refers to a group that is capable of penetrating a bodily membrane, e.g., a cell membrane, a nucleus membrane and the like. Cell membrane-permeable groups therefore provide cell membrane-penetrative or cell membrane-permeability characteristics to compounds that incorporate same and enable the penetration of such compounds into cells, nuclei and the like. Such delivering groups therefore serve for delivering substances into cells and/or cellular compartments.

Preferably, the cell membrane-permeable group is a cell membrane-permeable peptide. Preferably, the cell membrane-permeable peptide comprises or consists of one or more amino acids selected from lysine, arginine, tryptophan, phenylalanine, leucine, and isoleucine. Alternatively, the cell membrane-permeable peptide comprises or consists of alternating polar and nonpolar amino acids. Exemplary cell membrane-permeable peptides that may be used in the present invention are penetratin, transportan, HIV1-Tat-Peptide₄₈₋₆₀, HIV1-Rev-Peptide₃₄₋₅₀, antennapedia₄₃₋₅₈ and octaarginine.

Another exemplary cell membrane-permeable group that may be used in the present invention is choline or a moiety bound to choline.

Another example of a cell membrane-permeable group that may be used in the present invention is an acetoxymethyl (AM) ester derivative of a carboxylic acid or a moiety comprising one or more acetoxymethyl (AM) ester derivatives of a carboxylic acid.

As described above, R¹ is an analyte-responsive group capable of reacting with an analyte, wherein

if L is present and X is present, then X—R¹ is converted into a XH group upon reaction of R¹ with said analyte, or
if L is present and X is absent, then R¹ is converted into a π-donor group upon reaction of R¹ with said analyte, or
if L and Y are absent and R¹ is —B(Z)(Z′) or —B(Z″)₃⁻Kat⁺, then R¹ is converted into a —OH group, or
if L is absent and Y is —O—, then the —O—R¹ moiety is converted into a —OH group.

As described above, analyte-responsive group R¹ protects (or masks) the phenol functionality of the luminophore. This means that, as shown in Scheme 1, the reaction of R¹ with an analyte leads to an —O⁻ group at Y position, whereupon an electron is transferred from that phenolate group to the peroxide bond of the dioxetane moiety, thereby leading to a cleave off of groups R^{E} and R^{F} and to an excited species. That excited species then returns to its ground state by emitting a photon. Finally, the phenolate group is protonated thereby leading to a compound of Formula II.

For example, if Y and L are absent and R¹ is

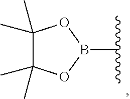 [SMILES: *B1OC(C)(C)C(C)(C)O1]

reaction of

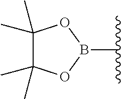 [SMILES: *B1OC(C)(C)C(C)(C)O1]

with a respective analyte (a peroxide, e.g. hydrogen peroxide, in this case), leads to a conversion of that boronate ester into a —OH group, i.e. to the formation of an —OH group at Y-position. That —OH group then undergoes deprotonation, electron transfer, cleave off of groups R^{E} and R^{F}, the formation of an excited species, which then return to its ground state by emitting a photon.
If, for example, Y is —O— and linker L is present, then a reaction of R¹ with an analyte leads to the conversion of X—R¹ (if X is present) or R¹ (if X is absent) into a π-donor (e.g., an —OH group), followed by a cleave off of linker L from the remainder part of the molecule and thereby to the formation of an —O⁻ group at Y-position, which then undergoes electron transfer and emissive return to its ground state by forming a compound of Formula II as described above. As one skilled in the art will recognize, moieties L1 to L8 are known self-immolative linker groups.
The compound of Formula Ib already comprises an —OH group at Y position (wherein L and R¹ are absent), because in case of the compound of Formula Ib, the carbon-carbon double bond represents the analyte-responsive part (more precisely a singlet oxygen-responsive part) of the compound.
A plethora of analyte responsive groups (e.g., enzyme-responsive groups, groups responsive to oxidation by peroxides, groups responsive to reduction) is known in the art and one skilled in the art will choose group R¹ according to his general knowledge depending on which analyte is to be detected.
Exemplary groups R¹, which may be used in the present invention are described in Table 1:

TABLE 1
sulfate, i.e.

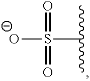 [SMILES: *S(=O)(=O)[O-]]

wherein preferably X is —O— if L is present, and Y is —O— if L is absent;
pyrophosphate diester disodium salt, i.e.

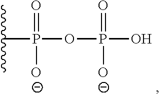 [SMILES: *P(=O)([O-])OP(=O)([O-])O]

wherein preferably X is —O— if L is present, and Y is —O— if L is absent;
phosphoethanolamine, i.e.

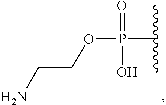 [SMILES: *P(=O)(O)OCCN]

wherein preferably X is —O— if L is present, and Y is —O— if L is absent;
elaidate, i.e.

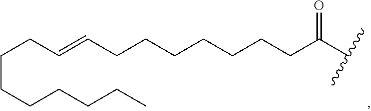 [SMILES: *C(=O)CCCCCCC/C=C/CCCCCCCC]

wherein preferably X is —O— if L is present, and Y is —O— if L is absent;
oleate, i.e.

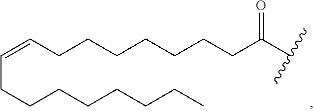 [SMILES: *C(=O)CCCCCCC/C=C\CCCCCCCC]

wherein preferably X is —O— if L is present, and Y is —O— if L is absent;
methyl ether;
ethyl ether;
benzyl ether;
2-deoxy-2-sulfamino-beta-D-glucopyranoside, i.e.,

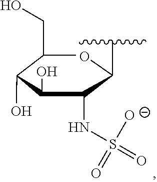 [SMILES: *[C@H](O)C(NS(=O)(=O)[O-])[C@H](O)[C@H](O)C(C)CO]

wherein preferably X is —O— if L is present, and Y is —O— if L is absent;
beta-D-glucoside-6-phosphoethanolamine, i.e.

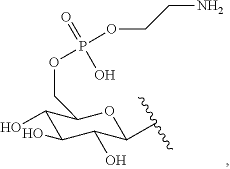 [SMILES: *C1OC(COP(=O)(O)OCCN)C(O)C(O)C1O]

wherein preferably X is —O— if L is present, and Y is —O— if L is absent;
N-acetyl-beta-D-glucosamine, i.e.

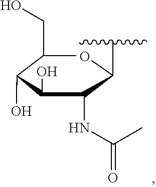 [SMILES: *[C@H](O)C(NC(C)=O)[C@H](O)[C@H](O)C(C)CO]

wherein preferably X is —O— if L is present, and Y is —O— if L is absent;
2-acetamido-2-deoxy-b-D-glucopyranoside-6-phosphocholine, i.e.,

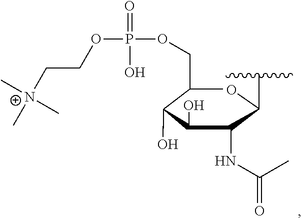 [SMILES: *[C@H](O)C(NC(C)=O)[C@H](O)[C@H](O)C(C)COP(=O)(O)OCC[N+](C)(C)C]

wherein preferably X is —O— if L is present, and Y is —O— if L is absent;
2-acetamido-2-deoxy-alpha-D-glucopyranoside-6-sulfate, i.e.

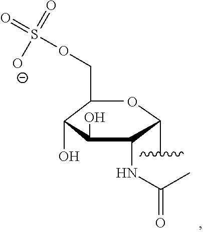 [SMILES: *[C@@H](O)C(NC(C)=O)[C@H](O)[C@H](O)C(C)COS(=O)(=O)[O-]]

wherein preferably X is —O— if L is present, and Y is —O— if L is absent;
2-acetamido-2-deoxy-4-O-(alpha-L-fucopyranosyl)-beta-D-glucopyranoside, i.e.

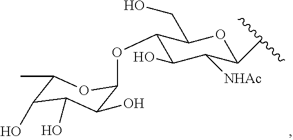 [SMILES: *C1OC(CO)C(OC2OC(C)C(O)C(O)C2O)C(O)C1NC(C)=O]

wherein preferably X is —O— if L is present, and Y is —O— if L is absent;
(6-thio-palmitoyl)-beta-D-glucopyranoside, i.e.,

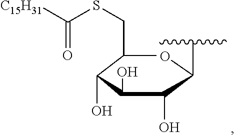

wherein preferably X is —O— if L is present, and Y is —O— if L is absent;
beta-D-lactoside, i.e.

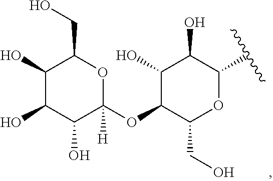 [SMILES: *[C@@H]1O[C@H](CO)[C@@H](O[C@@H]2O[C@H](CO)[C@H](O)[C@H](O)[C@H]2O)[C@H](O)[C@H]1O]

wherein preferably X is —O— if L is present, and Y is —O— if L is absent;
beta-D-galactopyranoside-6-sulfate, i.e.

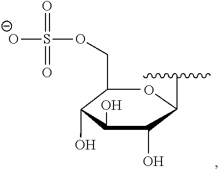 [SMILES: *[C@H](O)C(O)[C@H](O)[C@H](O)C(C)COS(=O)(=O)[O-]]

wherein preferably X is —O— if L is present, and Y is —O— if L is absent;
3-O-(alpha-L-fucopyranosyl)-beta-D-galactopyranoside, i.e.

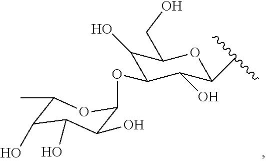 [SMILES: *C1OC(CO)C(O)C(OC2OC(C)C(O)C(O)C2O)C1O]

wherein preferably X is —O— if L is present, and Y is —O— if L is absent;
4-O-(alpha-L-fucopyranosyl)-beta-D-galactopyranoside, i.e.

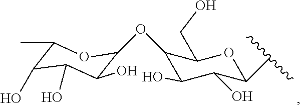 [SMILES: *C1OC(CO)C(OC2OC(C)C(O)C(O)C2O)C(O)C1O]

wherein preferably X is —O— if L is present, and Y is —O— if L is absent;
2-acetamido-2-deoxy-3,6-di-O-pivaloyl-beta-D-galactopyranoside, i.e.

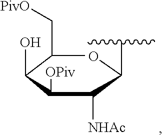

wherein preferably X is —O— if L is present, and Y is —O— if L is absent;
2-acetamido-2-deoxy-beta-D-galactopyranoside-4-sulfate, i.e.

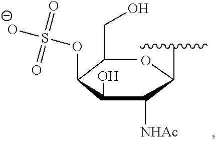 [SMILES: *[C@H](O)C(NC(C)=O)[C@H](O)[C@@H](OS(=O)(=O)[O-])C(C)CO]

wherein preferably X is —O— if L is present, and Y is —O— if L is absent;
alpha-D-mannopyranoside, i.e.

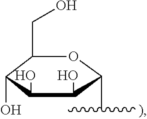

wherein preferably X is —O— if L is present, and Y is —O— if L is absent;
beta-D-mannopyranoside, i.e.

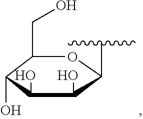

wherein preferably X is —O— if L is present, and Y is —O— if L is absent;
alpha-D-mannopyranoside 6-sulfate, i.e.

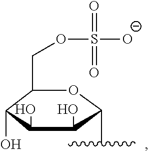

wherein preferably X is —O— if L is present, and Y is —O— if L is absent;
alpha-D-mannopyranoside-2-phosphoethanolamine, i.e.

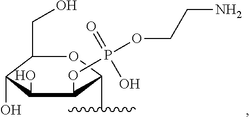

wherein preferably X is —O— if L is present, and Y is —O— if L is absent;
alpha-D-mannopyranoside-6-phosphoethanolamine, i.e.

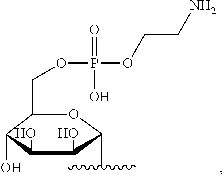

wherein preferably X is —O— if L is present, and Y is —O— if L is absent;
alpha-L-idopyranoside, i.e.

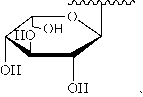

wherein preferably X is —O— if L is present, and Y is —O— if L is absent;
alpha-L-idopyranosiduronic acid, i.e.

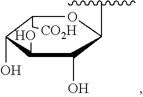

wherein preferably X is —O— if L is present, and Y is —O— if L is absent;
alpha-L-idopyranosiduronic acid 2-sulphate disodium salt, i.e.

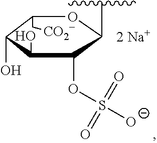

wherein preferably X is —O— if L is present, and Y is —O— if L is absent;
alpha-L-rhamnopyranoside, i.e.

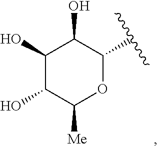 [SMILES: *[C@@H]1O[C@@H](C)[C@H](O)[C@@H](O)[C@H]1O]

wherein preferably X is —O— if L is present, and Y is —O— if L is absent;
a-D-N-glycolylneuraminic acid, i.e.

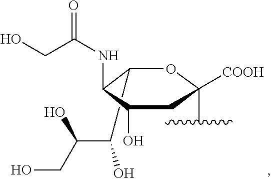

wherein preferably X is —O— if L is present, and Y is —O— if L is absent.
3-deoxy-D-glycero-a-D-galacto-2-nonulosonic acid, i.e.

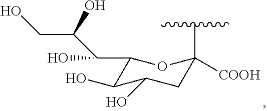 [SMILES: *C1(C(=O)O)CC(O)C(O)C([C@H](O)[C@H](O)CO)O1]

wherein preferably X is —O— if L is present, and Y is —O— if L is absent;
a-L-fucopyranoside, i.e.

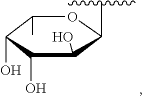

wherein preferably X is —O— if L is present, and Y is —O— if L is absent;
b-L-fucopyranoside, i.e.

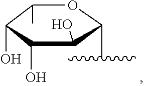

wherein preferably X is —O— if L is present, and Y is —O— if L is absent;
b-D-fucopyranoside, i.e.

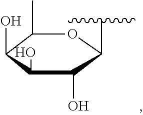

wherein preferably X is —O— if L is present, and Y is —O— if L is absent;
a-L-arabinofuranoside, i.e.

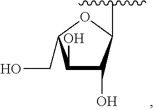

wherein preferably X is —O— if L is present, and Y is —O— if L is absent;
a-L-arabinopyranoside, i.e.

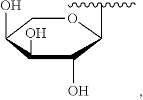

wherein preferably X is —O— if L is present, and Y is —O— if L is absent;
a-D-ribofuranoside, i.e.

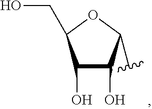

wherein preferably X is —O— if L is present, and Y is —O— if L is absent;
b-D-ribofuranoside, i.e.

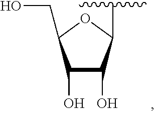

wherein preferably X is —O— if L is present, and Y is —O— if L is absent;
a-D-xylopyranoside, i.e.

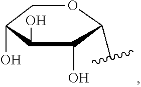

wherein preferably X is —O— if L is present, and Y is —O— if L is absent;
b-D-xylopyranoside, i.e.

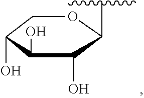

wherein preferably X is —O— if L is present, and Y is —O— if L is absent;
b-D-chitobioside, i.e.

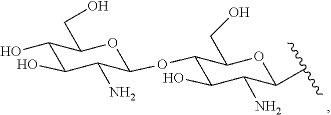 [SMILES: *C1OC(CO)C(OC2OC(CO)C(O)C(O)C2N)C(O)C1N]

wherein preferably X is —O— if L is present, and Y is —O— if L is absent;
4-deoxy-b-D-chitobioside, i.e.

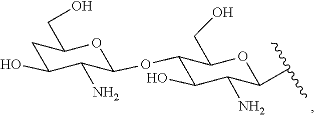 [SMILES: *C1OC(CO)C(OC2OC(CO)CC(O)C2N)C(O)C1N]

wherein preferably X is —O— if L is present, and Y is —O— if L is absent;
N,N-diacetyl-b-D-chitobioside, i.e.

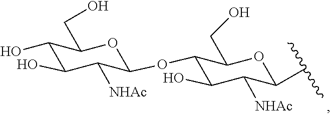 [SMILES: *C1OC(CO)C(OC2OC(CO)C(O)C(O)C2NC(C)=O)C(O)C1NC(C)=O]

wherein preferably X is —O— if L is present, and Y is —O— if L is absent;
N,N′,N″-triacetyl-b-D-chitotrioside, i.e.

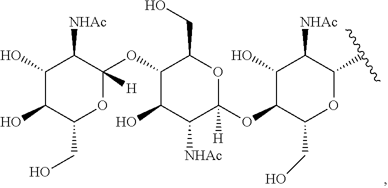 [SMILES: *[C@@H]1O[C@H](CO)[C@@H](O[C@@H]2O[C@H](CO)[C@@H](O[C@@H]3O[C@H](CO)[C@@H](O)[C@H](O)[C@H]3NC(C)=O)[C@H](O)[C@H]2NC(C)=O)[C@H](O)[C@H]1NC(C)=O]

wherein preferably X is —O— if L is present, and Y is —O— if L is absent;
N,N′,N″,N″-tetraacetyl-b-D-chitotetraoside, i.e.

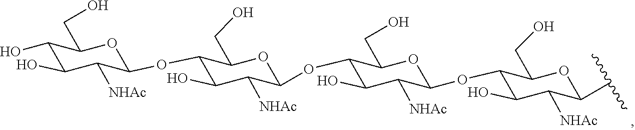 [SMILES: *C1OC(CO)C(OC2OC(CO)C(OC3OC(CO)C(OC4OC(CO)C(O)C(O)C4NC(C)=O)C(O)C3NC(C)=O)C(O)C2NC(C)=O)C(O)C1NC(C)=O]

wherein preferably X is —O— if L is present, and Y is —O— if L is absent;
b-D-cellotrioside, i.e.

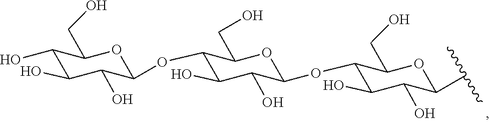 [SMILES: *C1OC(CO)C(OC2OC(CO)C(OC3OC(CO)C(O)C(O)C3O)C(O)C2O)C(O)C1O]

wherein preferably X is —O— if L is present, and Y is —O— if L is absent;
b-D-cellotetraoside, i.e.

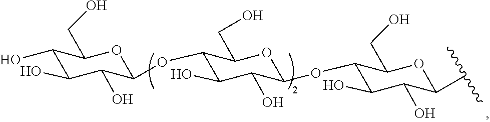

wherein preferably X is —O— if L is present, and Y is —O— if L is absent;
b-D-cellopentoside, i.e.

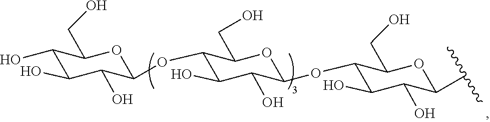

wherein preferably X is —O— if L is present, and Y is —O— if L is absent;
b-D-cellohexaoside, i.e.

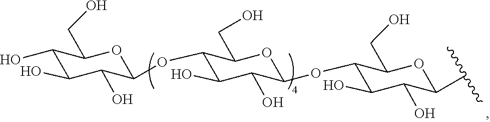

wherein preferably X is —O— if L is present, and Y is —O— if L is absent;
b-D-celloheptaoside, i.e.

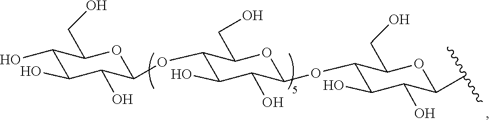

wherein preferably X is —O— if L is present, and Y is —O— if L is absent;
b-D-cellopolyoside, i.e.

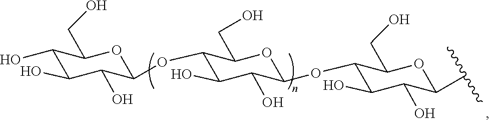

wherein n is 6, 7, 8, 9, 10, 11, 12, 13, 14, 15, or 16, and wherein preferably X
is —O— if L is present, and Y is —O— if L is absent;
b-D-gentiobioside, i.e.

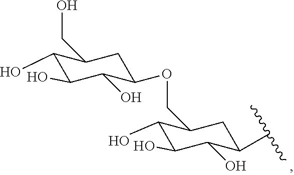 [SMILES: *C1CC(COC2CC(CO)C(O)C(O)C2O)C(O)C(O)C1O]

wherein preferably X is —O— if L is present, and
Y is —O— if L is absent;
b-D-gentiotrioside, i.e.

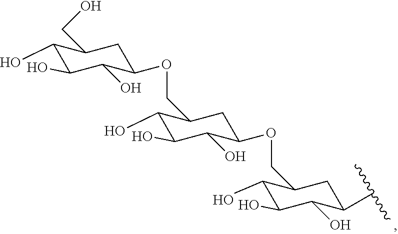 [SMILES: *C1CC(COC2CC(COC3CC(CO)C(O)C(O)C3O)C(O)C(O)C2O)C(O)C(O)C1O]

wherein preferably X is —O— if L is present, and Y is —O—
if L is absent;
Maltobioside, i.e.

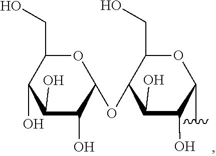

wherein preferably X is —O— if L is present, and Y is
—O— if L is absent;
Maltotrioside, i.e.

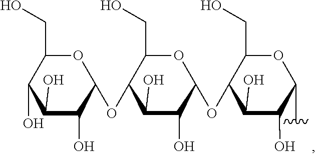

wherein preferably X is —O— if L is present, and
Y is —O— if L is absent;
Maltotetraoside, i.e.

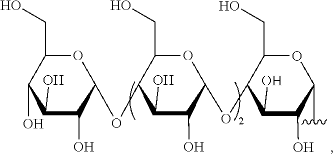

wherein preferably X is —O— if L is present, and
Y is —O— if L is absent;
Maltopentaoside, i.e.

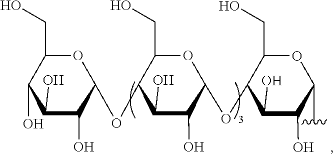

wherein preferably X is —O— if L is present, and
Y is —O— if L is absent;
Maltohexaoside, i.e.

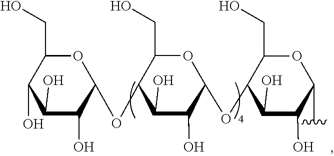

wherein preferably X is —O— if L is present, and
Y is —O— if L is absent;
Maltoheptaoside, i.e.

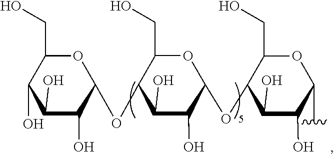

wherein preferably X is —O— if L is present, and
Y is —O— if L is absent;
Maltopolyoside, i.e.

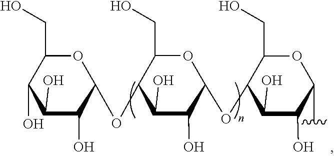

wherein n is 6, 7, 8, 9, 10, 11, 12, 13, 14, 15, or
16, and wherein preferably X is —O—
If L is present, and Y is —O— if L is absent;
b-D-xylobiosie, i.e.

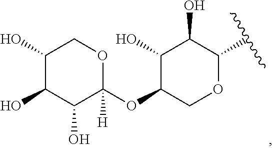 [SMILES: *[C@@H]1OC[C@@H](O[C@@H]2OC[C@@H](O)[C@H](O)[C@H]2O)[C@H](O)[C@H]1O]

wherein preferably X is —O— if L is present, and
Y is —O— if L is absent;
b-D-xylotrioside, i.e.

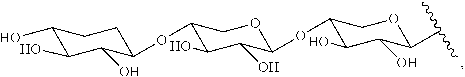 [SMILES: *C1OCC(OC2OCC(OC3CCC(O)C(O)C3O)C(O)C2O)C(O)C1O]

wherein preferably X is —O— if L is present, and
Y is —O— if L is absent;

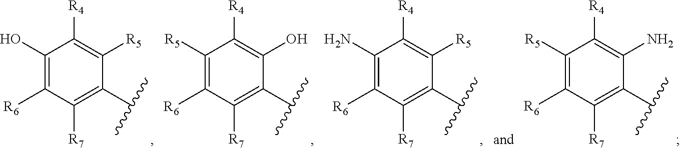

—B(Z)(Z′), —B(Z″)₃⁻ Kat⁺;
—NO₂;

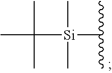 [SMILES: *[Si](C)(C)C(C)(C)C]

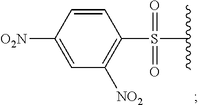 [SMILES: *S(=O)(=O)c1ccc([N+](=O)[O-])cc1[N+](=O)[O-]]

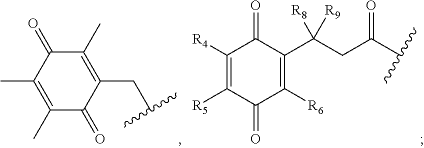

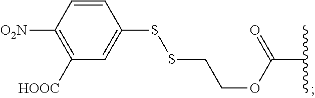 [SMILES: *C(=O)OCCSSc1ccc([N+](=O)[O-])c(C(=O)O)c1]

azide;

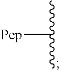

a group having the formula

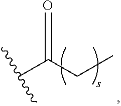

wherein s is 0 or an integer of from 1 to 18, preferably s is 0, 2, 6, 7, and wherein
preferably X is —O— if L is present, and Y is —O— if L is absent;
a group having the formula

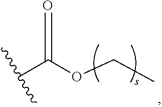

wherein s is 0 or an integer of from 1 to 18, preferably s is 1, and wherein preferably
is —NH— if L is present;
myo-inositol phosphoryl, wherein preferably X is —O— if L is present, and Y is
—O— if L is absent;
Phosphoryl, wherein preferably X is —O— if L is present, and Y is —O— if L is
absent;
amino acidyl having the formula

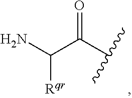

wherein R^{qr} is a side group depending on the respective amino acid,
wherein said amino acidyl is preferably selected from L-alaninyl,
L-leucinyl, and β-alanyl,and wherein X is preferably —NH— if L is present;
di-peptidyl having the formula

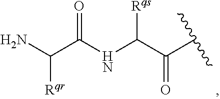

wherein R^{qr} and R^{qs} are side groups depending on the respective
amino acids of which the di-peptidyl group is composed of,
wherein X is preferably —NH— if L is present;
tri-peptidyl having the formula

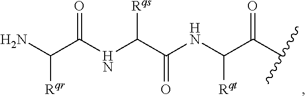

wherein R^{qr}, R^{qs}, and R^{qt} are side groups depending on the respective
amino acids of which the tri-peptidyl group is composed of,
wherein X is preferably —NH— if L is present;
L-pyroglutamic acidyl, i.e.

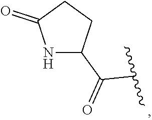 [SMILES: *C(=O)C1CCC(=O)N1]

wherein preferably X is —NH— if L is present;
glycosidyl;
di-saccharidyl;
an amino sugar moiety;
beta-D-galactopyranoside, i.e.

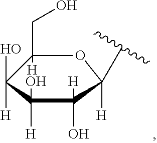

wherein preferably X is —O— if L is present, and Y is —O— if L is absent;
alpha-D-galactopyranoside, i.e.

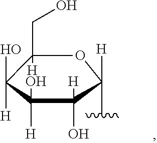

wherein preferably X is —O— if L is present, and Y is —O— if L is absent;
alpha-D-glucopyranoside, i.e.

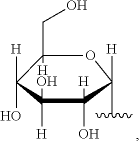

wherein preferably X is —O— if L is present, and Y is —O— if L is absent;
beta-D-glucopyranoside, i.e.

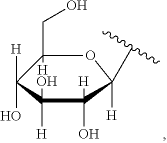

wherein preferably X is —O— if L is present, and Y is —O— if L is absent;
beta-D-glucuronyl, i.e.

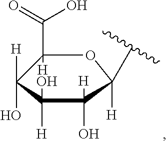

wherein preferably X is —O— if L is present, and Y is —O— if L is absent;
beta-D-glucuronyl sodium salt, i.e.

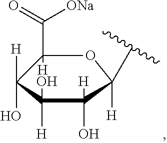

wherein preferably X is —O— if L is present, and Y is —O— if L is absent;
n-acetyl-beta-D-galactosaminidyl, i.e.

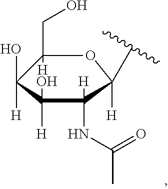

wherein preferably X is —O— if L is present, and Y is —O— if L is absent;
N-acetylneuraminidyl, i.e.

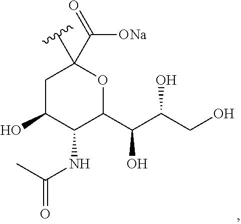

wherein preferably X is —O— if L is present, and Y is —O— if L is absent;
cellobioside, i.e.

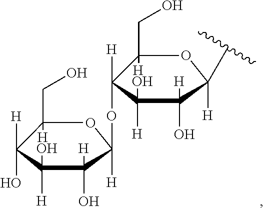

wherein preferably X is —O— if L is present;
choline phosphoryl, i.e.

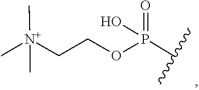 [SMILES: *P(=O)(O)OCC[N+](C)(C)C]

wherein preferably X is —O— if L is present;
oxalylester having the formula

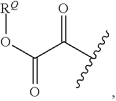

wherein R^{Q} is an optionally substituted C₁—C₁₂ alkyl group,
wherein X is preferably —NH— if L is present;
Boc-Val-Pro-Argininyl;
Boc-Asp(OBzl)-Pro-Argininyl;
SucOMe-Arg-Pro-Tyrosinyl (SucOMe-RPY-);
a beta-lactamase-labile group, preferably a beta-lactam antibiotic, more preferably a
penicillin, a cephalosporin of generation 1 to 5, a cephamycin, or a carbapenem;
Ac-QLQ-;
Ac-FQLQ-;
Ac-EFQLQ-;
Ac-DEFQLQ-;
amides of 5-substituted-o-antranilic acid methyl ester, wherein preferably X is
absent if L is present;
acrylic acid ester, wherein preferably X is —O— if L is present;
L-alanyl (A-);
L-leucinyl (L-);
β-alanyl;

Particularly preferred beta-lactamase-labile groups are selected from the group consisting of

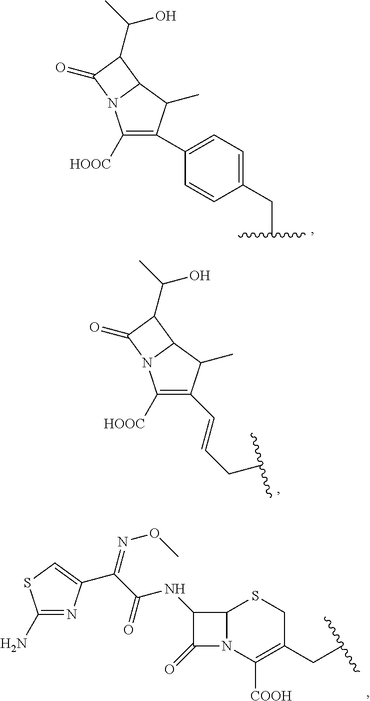 [SMILES: *Cc1ccc(C2=C(C(=O)O)N3C(=O)C(C(C)O)C3C2C)cc1 ; *C/C=C/C1=C(C(=O)O)N2C(=O)C(C(C)O)C2C1C ; *CC1=C(C(=O)O)N2C(=O)C(NC(=O)/C(=N\OC)c3csc(N)n3)C2SC1]

preferably

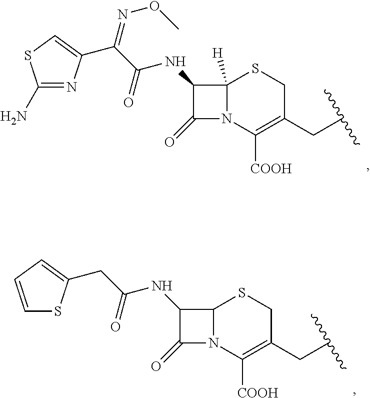 [SMILES: *CC1=C(C(=O)O)N2C(=O)[C@@H](NC(=O)/C(=N\OC)c3csc(N)n3)[C@H]2SC1 ; *CC1=C(C(=O)O)N2C(=O)C(NC(=O)Cc3cccs3)C2SC1]

preferably

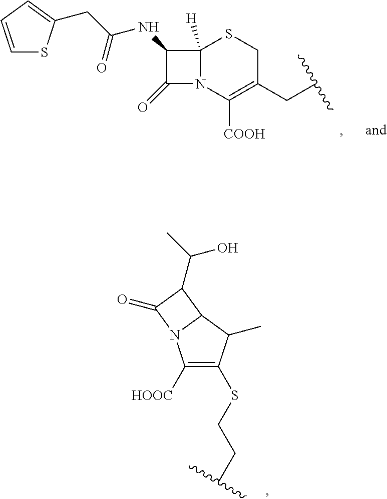 [SMILES: *CC1=C(C(=O)O)N2C(=O)[C@@H](NC(=O)Cc3cccs3)[C@H]2SC1 ; *CCSC1=C(C(=O)O)N2C(=O)C(C(C)O)C2C1C]

preferably

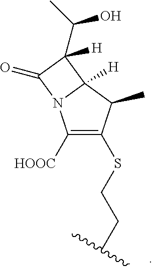 [SMILES: *CCSC1=C(C(=O)O)N2C(=O)[C@H]([C@@H](C)O)[C@H]2[C@H]1C]

When R¹ is

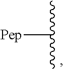

then L is present and X is —NH— or —NR^{G}—, preferably —NH—. Pep is a group comprising a peptide moiety consisting of at least two amino acid residues and linked to L via a carboxylic acid group of said peptide moiety.

R₄, R₅, R₆, and R₇ are independently selected from hydrogen; C1-C6 alkyl, preferably methyl; halogen, preferably fluorine and chlorine; alkoxy, preferably methoxy; and cyano. R₈ and R₉ are independently selected from C1-C4 alkyl, preferably methyl, or H, wherein R₈ and R₉ are preferably both methyl.

Preferably,

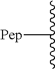

is

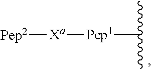

wherein
Pep¹ is a protease cleavable peptide moiety consisting of at least two amino acid residues and linked via a carboxylic group thereof to L, wherein said protease cleavable peptide moiety is optionally protected or linked through an amino group thereof to a PEG-containing group; X^{a} is absent, or is a linker linked to Pep¹ via an amide bond through either a carboxyl or amino group of Pep¹; and Pep² is absent, or a cell-penetrating peptide moiety linked to X^{a} either via an amide bond through an amino or carboxyl group thereof, or through a thiol group thereof, provided that X^{a} and Pep² are both either absent or present, and when Pep¹ is protected or linked to a PEG-containing group, X^{a} and Pep² are absent.

More preferably, Pep¹ is a peptide moiety comprising the amino acid sequence Val-Cit, Phe-Lys, Gly-Phe-Leu-Gly, Gly-Gly-Pro-Nle, Ala-Ala-Asn or His-Ser-Ser-Lys-Leu-Gln, wherein said amino acid sequence is linked via the carboxylic group of the citrulline, lysine, glycine, norleucine, asparagine or glutamine, respectively, to L; and optionally protected at an amino group thereof, or linked via an amide bond and through said amino group to a PEG-containing group, wherein preferably said PEG-containing group is a group of formula

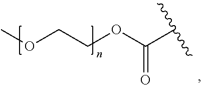

wherein n is an integer of 1 to 227

Even more preferably, Pep¹ is a peptide moiety comprising the amino acid sequence Val-Cit, Phe-Lys, Gly-Phe-Leu-Gly, Gly-Gly-Pro-Nle, Ala-Ala-Asn or His-Ser-Ser-Lys-Leu-Gln, linked via the carboxylic group of the citrulline, lysine, glycine, norleucine, asparagine or glutamine, respectively, to L; X^{a} is a linker linked to Pep¹ via an amide bond through either a carboxyl or amino group of Pep¹; and Pep² is a peptide moiety linked to X^{a} through a thiol group thereof, wherein preferably X^{a} is a linker of the formula

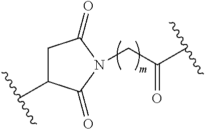

linked to Pep¹ via an amide bond through an amino group of Pep′, wherein m is an integer of 1-20, and the alkylene chain of X^{a} is optionally interrupted with one or more —O— groups; and Pep² is a peptide moiety of the sequence Cys-Gly-Lys-Arg-Lys, linked to X^{a} through the thiol group of the cysteine residue.

Preferably, R¹ is selected from the group consisting of

| —B(Z)(Z′), | —B(Z″)₃⁻ Kat⁺, | —NO₂, |
|---|---|---|
| azide (—N3), | 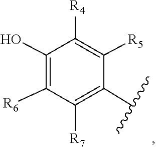 | 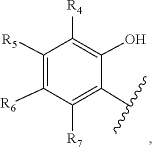 |
| 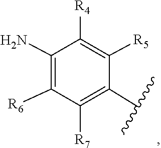 | 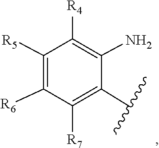 | 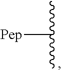 |
| amino acidyl having the | di-peptidyl having the | tri-peptidyl having the |
| formula | formula | formula |
| 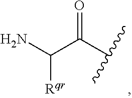 | 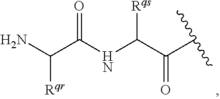 | 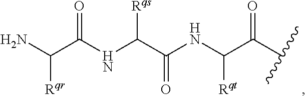 |
| wherein R^{qr} is a side group | wherein R^{qr} and R^{qs} are side | wherein R^{qr}, R^{qs}, and R^{qt} |
| depending on the | groups depending on the | are side groups depending |
| respective amino acid, | respective amino acids of | on the respective amino |
| wherein said amino acidyl | which the di-peptidyl group | acids of which the tri- |
| is preferably selected from | is composed of, wherein X | peptidyl group is |
| L-alaninyl, L-leucinyl, and | is preferably —NH— if L is | composed of, wherein X is |
| β-alanyl, and wherein X is | present, | preferably —NH— if L is |
| preferably —NH— if L is | | present, |
| present, | | |
| SucOMe-Arg-Pro-Tyrosinyl | L-alanyl (A-), | L-leucinyl (L-), |
| (SucOMe-RPY-), | | |
| β-alanyl, and | beta-D-galactopyranoside | |
| | 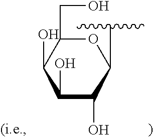 | |
| | (preferably X is —O— if L is | |
| | present, and Y is —O— if L is | |
| | absent) | |

As one skilled in the art will understand, a positive charge of the compound according to Formula Ia or Ib, e.g. the positive charge resulting from charged group R², is balanced by a counter anion. Thus, in a preferred embodiment, in case the compound of Formula Ia or Ib comprises a positive charge, the compound of Formula Ia or Ib further comprises an anion balancing the positive charge, wherein said anion is preferably selected from the group consisting of a fluoride, chloride, bromide, iodide, and CF₃SO₃⁻. However, as one skilled in the art will recognize, a specific counter anion cannot always be assigned to a specific positive charge. This is in particular the case when the compound of Formula Ia or Ib is used, e.g., for detecting the presence of an analyte. This is, because in this case, the compound of Formula Ia or Ib will be present in a liquid medium, e.g. a ready-to-use injectable solution, where the counter anion balancing the positive charge is solvated and located in random vicinity to the positive charge of group R². Even more, since the counter anion is solvated and located in random vicinity to the positive charge when the inventive compounds are actually used for detecting a specific analyte, the counter anion does not affect the performance of the inventive compounds. Therefore, it is not intended to limit the claimed invention by any specific counter anion.

The same applies, mutatis mutandis, for any net negative charge of a compound of Formula Ia or lb. That means that any net negative charge is balanced by a counter cation. Preferred counterions balancing a negative charge are ammonium, ammonium derivatives such as cyclohexyammonium, para-toluidinium, Li⁺, Na⁺, K⁺, Ca²⁺, and Mg⁺. It is also to be understood that a counterion balancing a positive or negative charge does not have to be an additional compound/ion that is different from the compound of Formula Ia or Ib but may also be part of the compound of Formula Ia or lb. Thus, the compound of Formula Ia or Ib may also be present in zwitterionic form. In the context of the present invention a “zwitterion” is a molecule with two or more functional groups, of which at least one has a positive and one has a negative electrical charge and the net charge of the entire molecule is zero.

Group R¹ may also be present in charged form. In this case one skilled in the art will understand that also this charge is balanced by a respective counterion. For example, if R¹ is a negatively charged group, this charge may be balanced by the positive charge of charged group R². Or in other words, if R¹ is negatively charged, the compound of Formula Ia or Ib may be preferably present as a zwitterion. It is, however, also within the scope of the present invention that the charge of a charged group R¹ is balanced by a counterion that is different from charged group R². However, also this counterion will be solvated and located in random vicinity to charged group R¹ in aqueous media and, therefore, also this counterion does not affect the overall performance of the inventive compounds. Hence, it is also not intended to limit the claimed invention by any specific counter ion of group R¹. Nonetheless, preferred counterions balancing the charge of a negatively charged group R¹ are ammonium, ammonium derivatives such as cyclohexyammonium, para-toluidinium, Li⁺, Na⁺, K⁺, Ca²⁺, and Mg²⁺, and particularly preferred counterions balancing the charge of a positively charged group R¹ are fluoride, chloride, bromide, and iodide.

Preferably, M is present.

As regards R² and the definition that

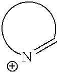

denotes a mono- or polycyclic, aromatic or nonaromatic ring system comprising the moiety

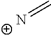

as a ring member, one skilled in the art understands that although moiety —B-M is omitted in

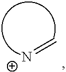

said moiety —B-M is connected to the positively chargen nitrogen atom in actual group R². Further, one skilled understands that moiety

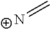

is an excerpt from the ring system as defined in claim 1, wherein, in the actual ring system, N is connected to —B-M and another ring member and the carbon atom on the right hand side of that moiety is connected to other ring member(s).

Preferably,

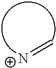

denotes a mono-, bi- or tricyclic, aromatic or nonaromatic ring system comprising the moiety

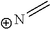

as a ring member. More preferably,

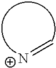

denotes a monocyclic, bicyclic, or tricyclic aromatic ring comprising the moiety

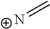

as a ring member.

Preferably, R² is selected from the group consisting of

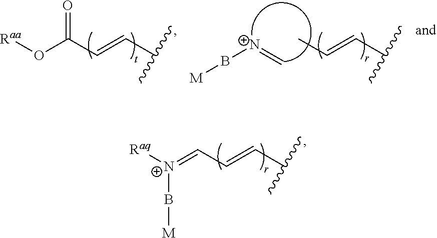

more preferably R² is selected from

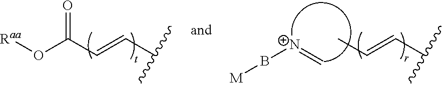

wherein any of the above-mentioned definitions of R^{aa}, R^{aq}, B, M, t and r may be applied. According to a preferred embodiment, R² is

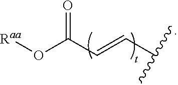

According to another preferred embodiment, R² is

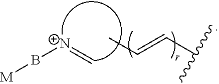

As set out above,

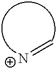

may be substituted with one or two negatively charged substituent(s), preferably selected from —COO⁻ and —SO₃⁻, in ortho position to the positively charged nitrogen atom.

It has been found that the positively charged nitrogen atom can be stabilized by introducing one or two negatively charged substituents, in particular —COO⁻ and —SO₃⁻, in ortho position to the positively charged nitrogen atom, thereby leading to increased luminescence intensities.

Further, if M is absent, B is —H and one or two ortho positions relative to the positively charged nitrogen ring atom are substituted with a —COO⁻ moiety, the respective moiety R² can function as a ligand for forming chelate complexes thereby stabilizing the positively charged nitrogen atom thereby leading to increased luminescence intensities.

Thus, in one preferred embodiment, M is absent and B is —H and R² comprises one or more negatively charged substituents in ortho position to the positively charged nitrogen atom, wherein said negatively charged substituents are preferably selected from the group consisting of —COO⁻ and —SO₃⁻.

Even more preferably, R² is selected from the group consisting of

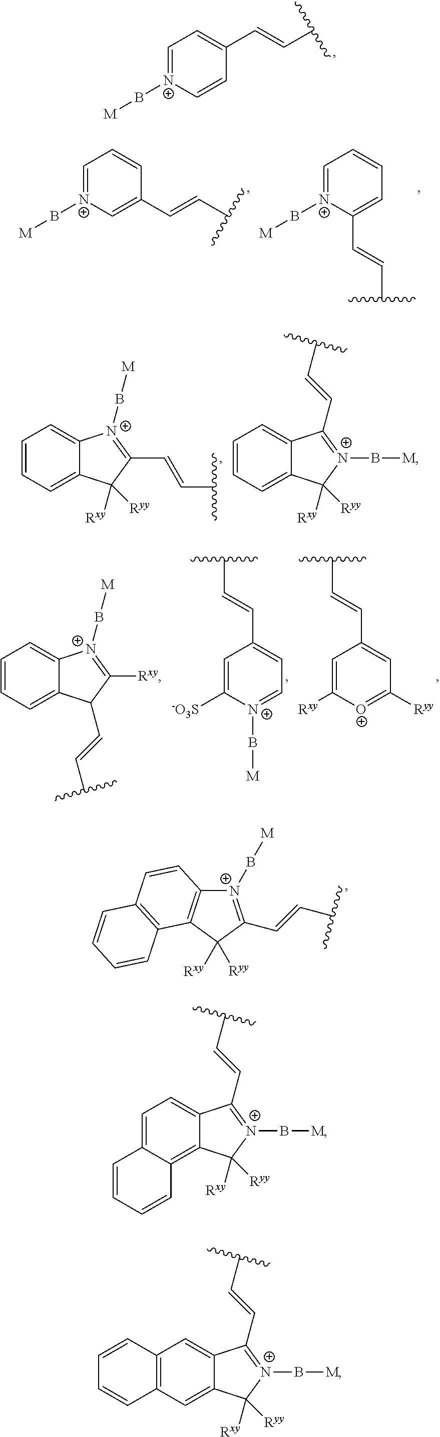

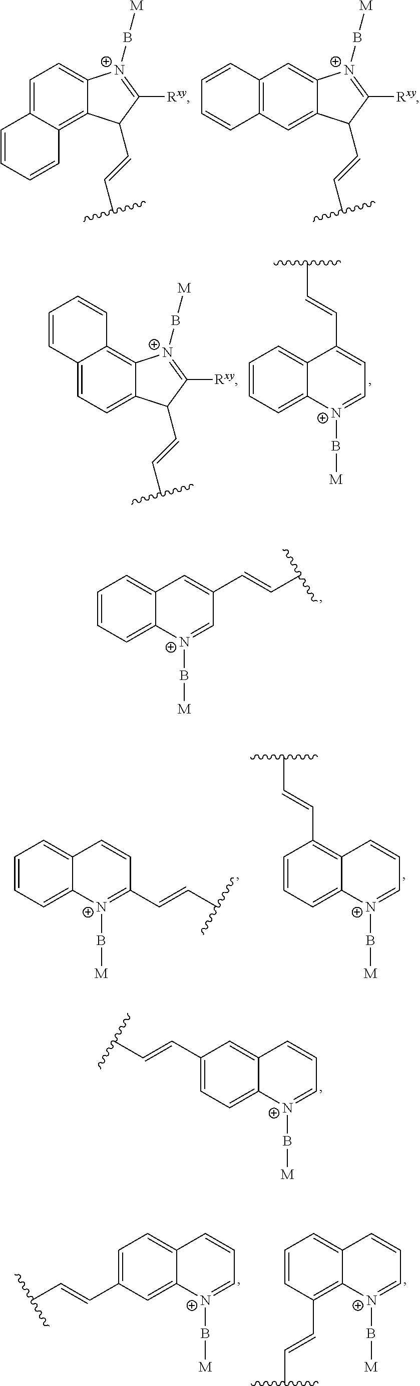

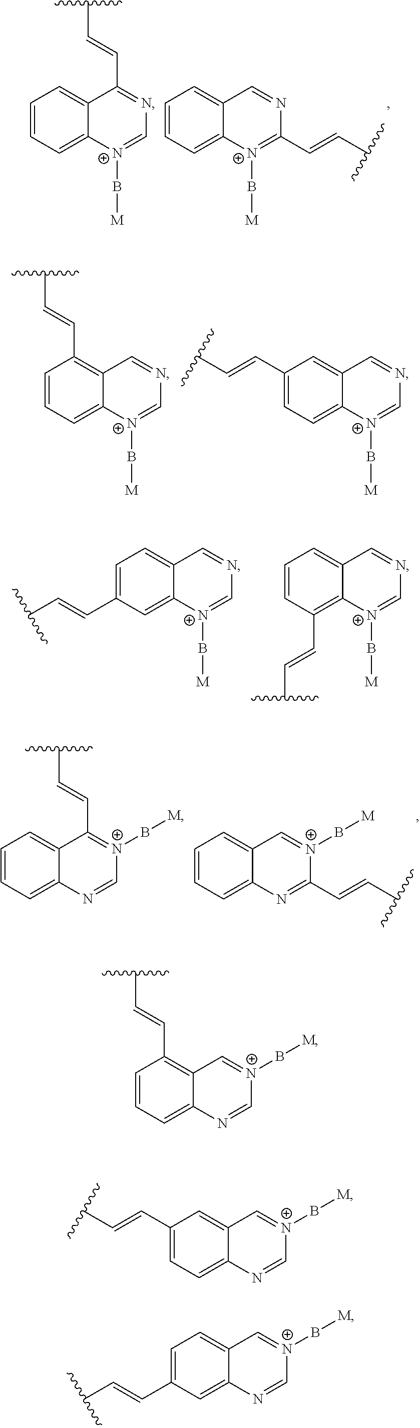

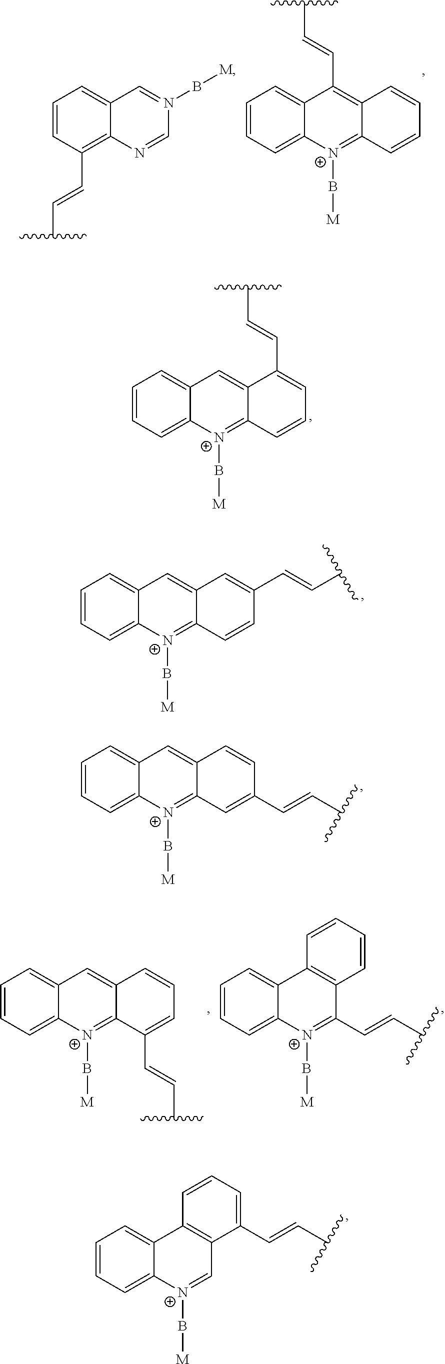

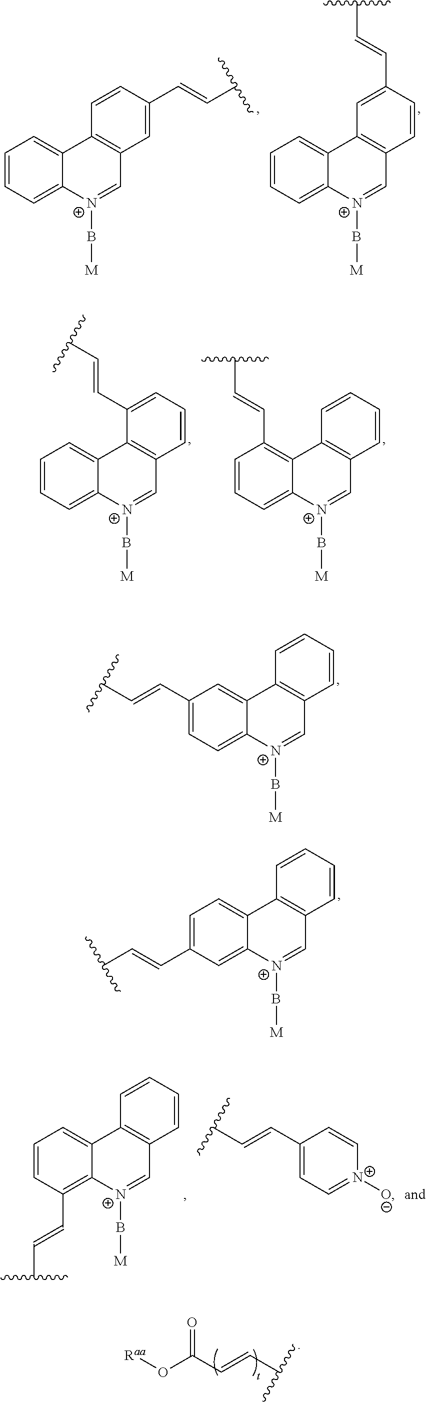

If possible, that means if the respective position is available for substitution, the aromatic ring is optionally substituted with one or two negatively charged substituent(s), preferably selected from −COO⁻ and —SO₃⁻, in ortho position to the positively charged nitrogen atom.

Preferably, R² is selected from the group consisting of

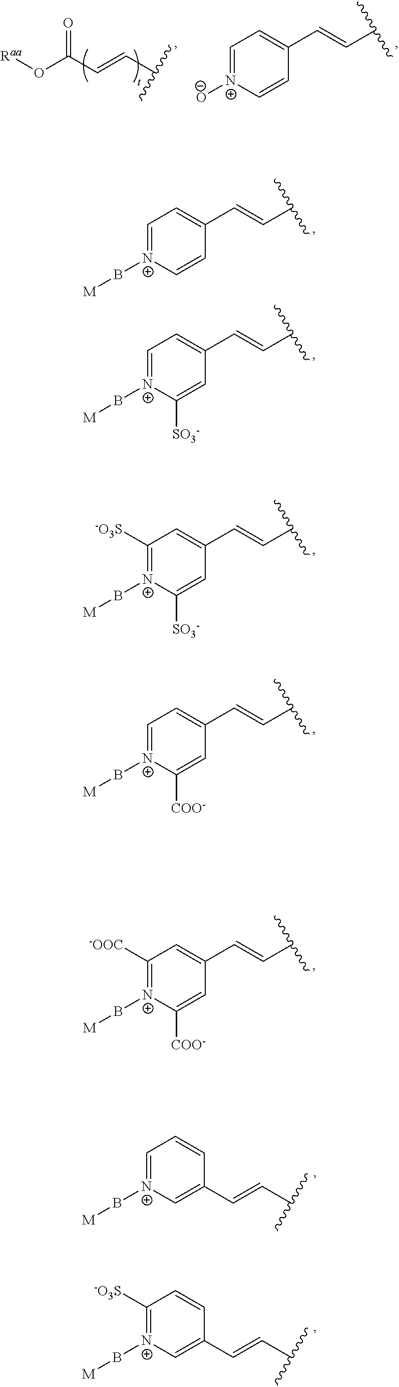

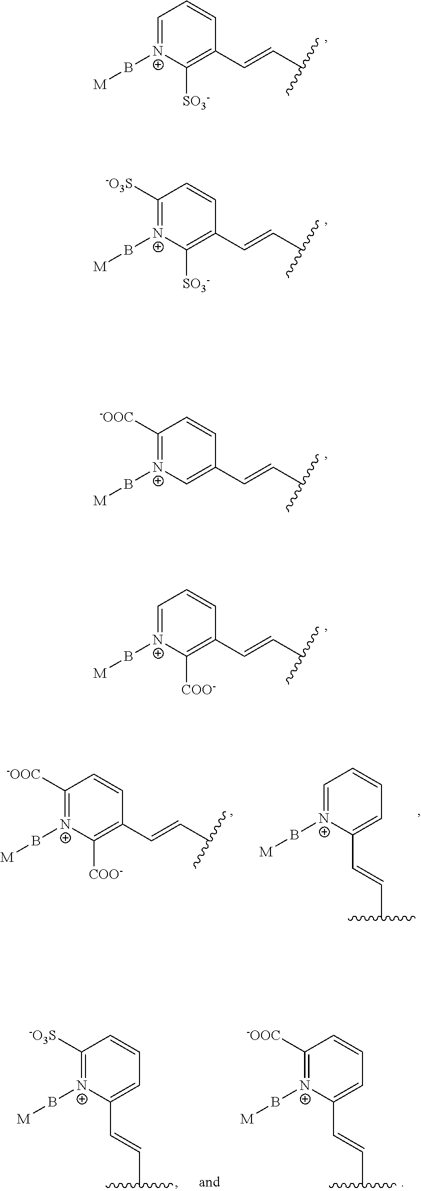

wherein R^{aa}, t, M and B are as defined above. Preferably R² is

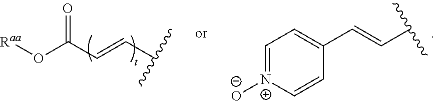

According to another preferred embodiment, R² is selected from the group consisting of

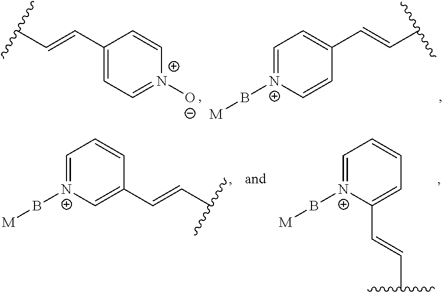

preferably

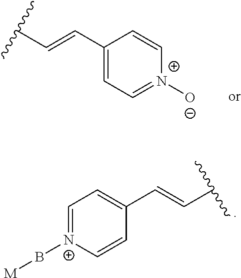

In a particularly preferred embodiment, R² is

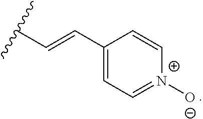 [SMILES: */C=C/c1cc[n+]([O-])cc1]

In another preferred embodiment, R² is

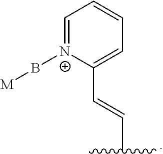

In another preferred embodiment, R² is

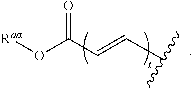

In another preferred embodiment, R² is selected from the group consisting of

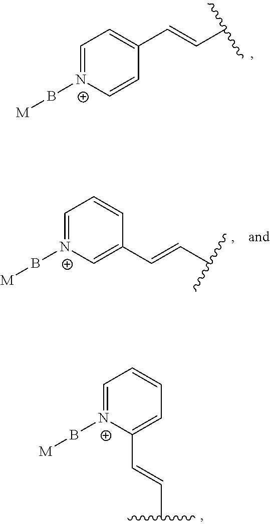

preferably

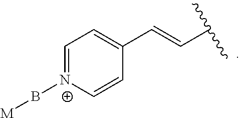

In another preferred embodiment, R² is

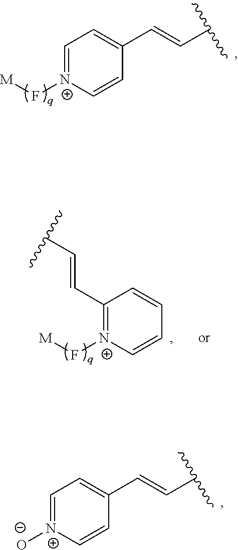

preferably

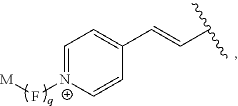

wherein F is a linear or branched C1 to C8 alkyl, preferably C2 to C6 alkyl, a linear or branched C2 to C8 alkenyl or a linear or branched C2 to C8 alkynyl chain, and wherein q is 2, 3, 4, 5, 6, 7, 8, 9, 10, or 11, preferably 3, 4, 5, 6, or 7, more preferably 5.
More preferably, R² is

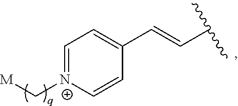

wherein q is 2, 3, 4, 5, 6, 7, 8, 9, 10, or 11, preferably 3, 4, 5, 6, or 7, more preferably 5.

In another preferred embodiment, R² is selected from the group consisting of

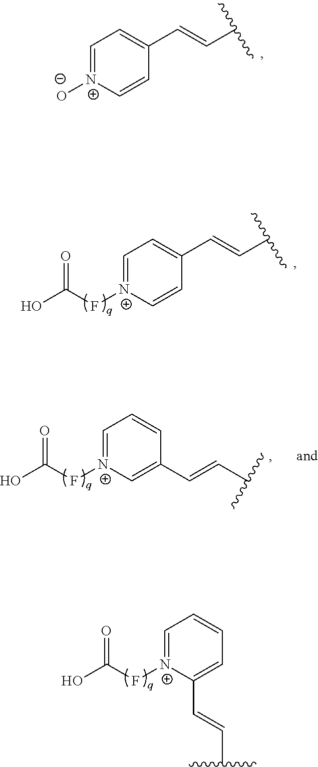

preferably from the group consisting of

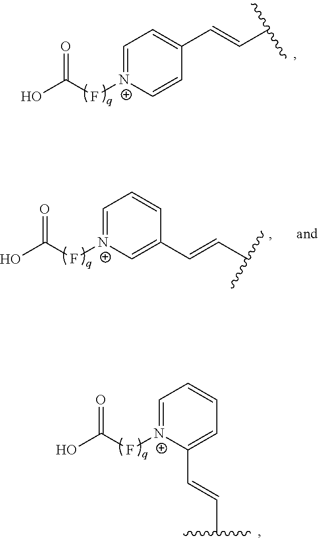

wherein F is a linear or branched C1 to C8 alkyl, preferably C2 to C6 alkyl, a linear or branched C2 to C8 alkenyl or a linear or branched C2 to C8 alkynyl chain, and wherein q is 2, 3, 4, 5, 6, 7, 8, 9, 10, or 11, preferably 3, 4, 5, 6, or 7, more preferably 5, and wherein, if possible, the aromatic ring is optionally substituted with one or two negatively charged substituent(s), preferably selected from −COO⁻ and —SO₃⁻, in ortho position to the positively charged nitrogen atom.

In a more preferred embodiment, R² is selected from the group consisting of

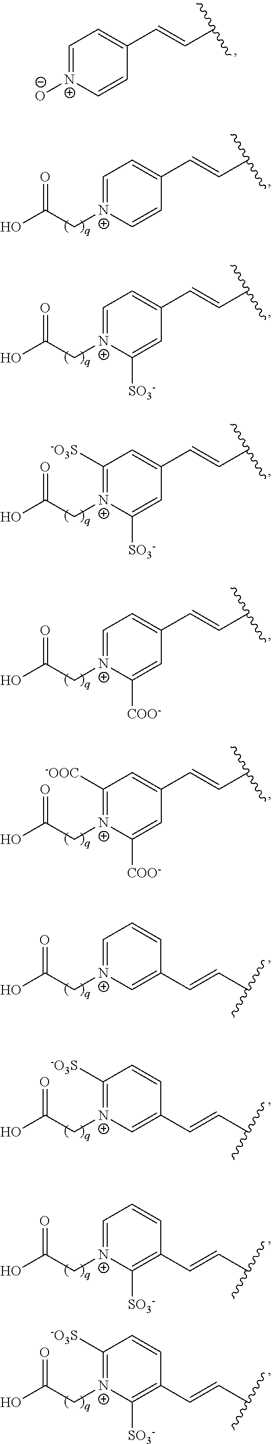

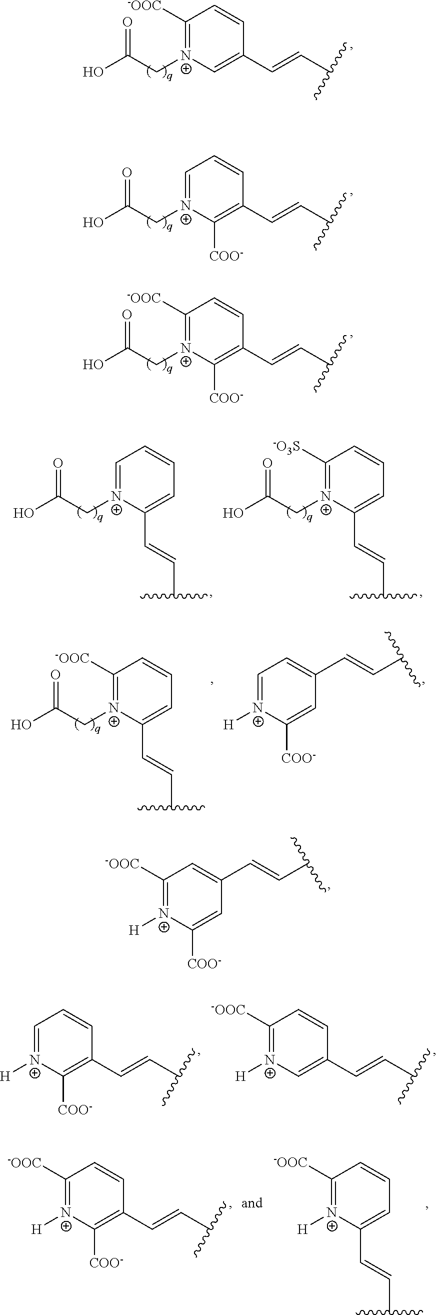

wherein q is 2, 3, 4, 5, 6, 7, 8, 9, 10, or 11, preferably 3, 4, 5, 6, or 7, more preferably 5

In an even more preferred embodiment, R² is selected from the group consisting of

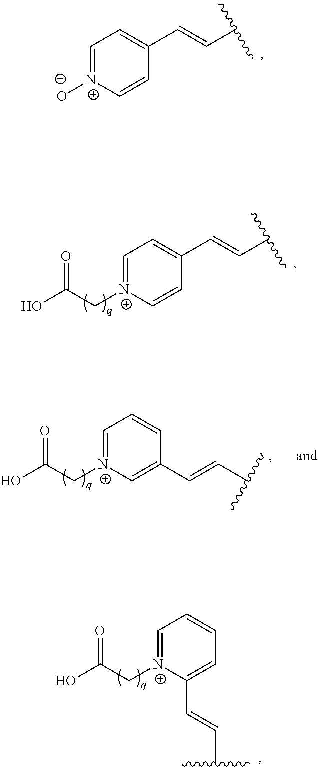

preferably from the group consisting of

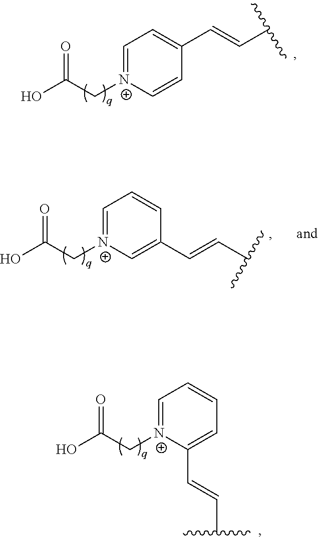

wherein q is 2, 3, 4, 5, 6, 7, 8, 9, 10, or 11, preferably 3, 4, 5, 6, or 7, more preferably 5.

It is preferred that M is present and B is —(CH₂)_{z}—, wherein z is 1-6, preferably 3-5, more preferably, 4 or 5, even more preferably 5. More preferably, B is —(CH₂)₂₋₆— and M is —COOH, even more preferably, B is —(CH₂)₅— and M is —COOH.

In each instance of R² defined above, the aromatic ring(s) of R² may be substituted with one or more groups selected from —OH, —CN, —SO₃⁻, linear or branched C1-C6 alkyl, linear or branched C2-C6 alkenyl, and linear or branched C2-C6 alkynyl, a polyethylene glycol chain or a polypropylene glycol chain.

In one embodiment, however, the aromatic ring(s) of R² are unsubstituted.

According to a preferred embodiment, R² is

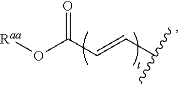

wherein t is 2, 3, or 4; and R^{aa} is —H, a linear or branched C1-6 alkyl, preferably methyl or ethyl, more preferably methyl, a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group such as

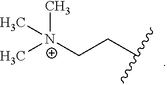 [SMILES: *CC[N+](C)(C)C]

Preferred moieties derived from an amino acid, a monosaccharide, a disaccharide, a polycarboxylic acid, polyethylene glycol, polypropylene glycol, or a polyol are described above. Preferred cell membrane-permeable groups that may be used in the present invention are also described above.

The moiety derived from an amino acid is preferably derived from arginine, histidine, lysine, aspartic acid, glutamic acid, serine, threonine, asparagine, glutamine, cysteine, glycine and proline. More preferably, the amino acid is selected from arginine, histidine, lysine, aspartic acid, and glutamic acid, more preferably from aspartic acid.

The moiety derived from a monosaccharide or a disaccharide is preferably derived from glucose, galactose, fructose, xylose, sucrose, lactose, maltose, and trehalose.

The moiety derived from a polycarboxylic acid is preferably derived from malic acid, 1,2,3,4-butanetetracarboxylic acid, citric acid, isocitric acid, succinic acid, methylsuccinic acid, itaconic acid, mesaconic acid, citraconic acid, tartaric acid, aconitic acid, propane-1,2,3-tricarboxylic acid, oxalic acid, malonic acid, glutaric acid, adipic acid, pimelic acid, suberic acid, azelaic acid, sebacic acid, maleic acid, fumaric acid, glutaconic acid, tartronic acid, mesoxalic acid, oxaloacetic acid, aspartic acid, α-hydroxy glutaric acid, arabinaric acid, acetonedicarboxylic acid, α-ketoglutaric acid, glutamic acid, diaminopimelic acid, saccharic acid, EDTA, nitrilotriacetic acid, EGTA, and ethylenediamine-N,N′-disuccinic acid (EDDS).

The moiety derived from a polyol is preferably derived from a sugar alcohol such as ethylene glycol, glycerol, erythritol, threitol, arabitol, xylitol, ribitol, mannitol, sorbitol, galactitol, fucitol, iditol, inositol, volemitol, isomalt, maltitol, lactitol; pentaerythritol, 1,3-propanediol, 1,2,4-butanetriol, 1,2,3-butanetriol, and 1,1,1-Tris(hydroxymethyl)ethane.

It is understood that the moieties derived from an amino acid, a monosaccharide or a disaccharide, a polycarboxylic acid, polyethylene glycol or polypropylene glycol, or a polyol form an ester functional group together with the —COO-part of said group R². That is, the atom attached to the following highlighted oxygen atom

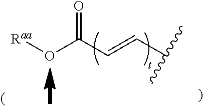

is a carbon atom.

It has surprisingly been found that group R² of formula

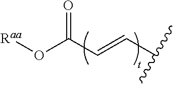

leads to extremely strong long-wavelength emission, depending on the number of conjugated double bonds.
Specifically, it has been found that group R² of formula

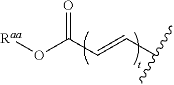

wherein t is 2 and R^{aa} is H leads to an emission with a maximum located at about 555 nm and that this emission maximum can be shiftet about 55 nm to longer wavelengths by converting the free carboxylic acid (i.e., R^{aa} is H) into an ester group, e.g. a methyl ester or a malic acid ester. Thus, if t is 2, R^{aa} is preferably not H, more preferably a moiety forming an ester group together with the —COO— part of said group R².

Further, it has been shown that the emission is shifted about 40 nm to longer wavelengths by introducing a further double bond (i.e. increasing t from 2 to 3). Thus, if t is 3 and R^{aa} is H, the emission maximum is located at about 595 nm. If t is 4 and R^{aa} is H, the emission maximum is expected to be located at about 635 nm. Again, the emission maximum can be further shiftet about 55 nm to longer wavelengths by converting the free carboxylic acid (i.e., R^{aa} is H) into an ester group.

However, since double bonds are of hydrophobic nature, compounds comprising a high number of said double bonds may suffer from solubility issues. Thus, if t is 3 or 4, it is preferred that R^{aa} is a hydrophilic group such as —H (in this case, the free carboxylic acid is referred to as the hydrophilic group), a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group such as

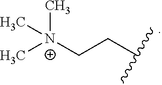 [SMILES: *CC[N+](C)(C)C]

A specifically preferred group R^{aa} is

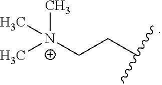 [SMILES: *CC[N+](C)(C)C]

It has been found that this group leads to good solubility in aqueous media and provides cell membrane-permeability while at the same time leading to strong long-wavelength emission.
In particular, it has surprisingly been found that group R² of formula

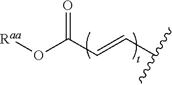

provides for remarkably strong luminescence intensity. For example, it has been found that a compound of the present invention bearing group R² of formula

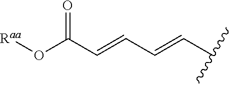

is about 47 times more emissive than a compound of the present invention bearing group R² of formula

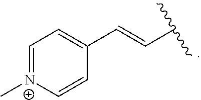 [SMILES: */C=C/c1cc[n+](C)cc1]

According to a preferred embodiment, t is 2. According to another preferred embodiment, t is 3. According to another preferred embodiment, t is 4.

Preferably, R³ is H, F, Cl, Br, I, CF₃, or R².

In one preferred embodiment, R^{A} and R^{C} are selected from H, F, Cl, Br, I, CF₃, and R².

In another preferred embodiment, R¹ is —B(Z)(Z′) or —B(Z″)₃⁻Kat⁺. More preferably R¹ is —B(Z)(Z′).

Preferably, —B(Z)(Z′) is —B(OH)₂ or

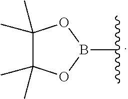 [SMILES: *B1OC(C)(C)C(C)(C)O1]

Preferably, R¹ is —B(OH)₂ or

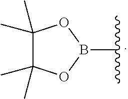 [SMILES: *B1OC(C)(C)C(C)(C)O1]

Preferably, L is present. Even more preferably, L is

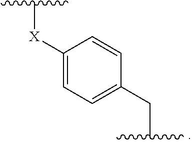

Preferably, Y is —O—.

Preferably, R^{A} or R^{C} is R², more preferably R^{A} is R².

Preferably, R³ is Cl, R^{A} is R², and R^{C} is H.

Preferably, M is —COOH.

Preferably, R^{C} is H and R^{A} is

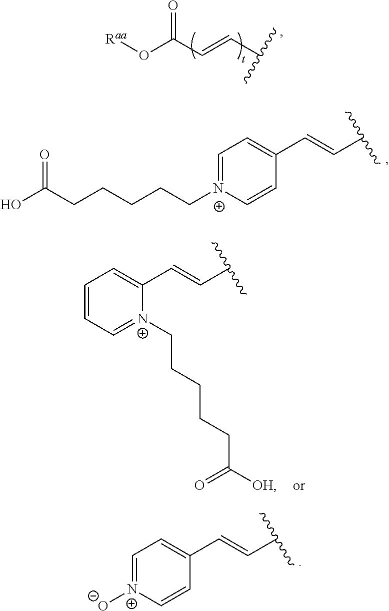

More preferably, R^{C} is H, R^{A} is

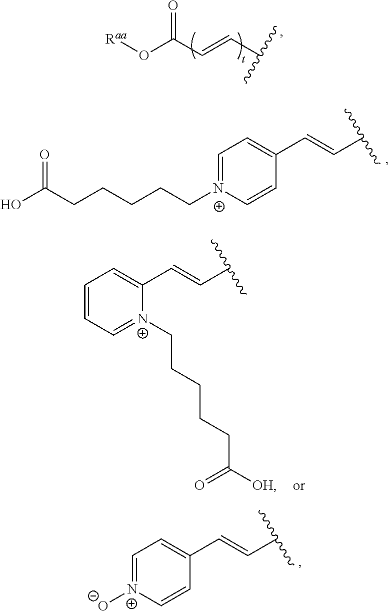

preferably

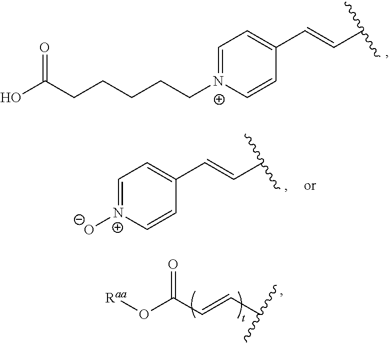

and R^{E} and R^{F} together with the carbon atom to which they are attached form optionally substituted adamantyl.

According to one particularly preferred embodiment, the compound of Formula Ia has the structure

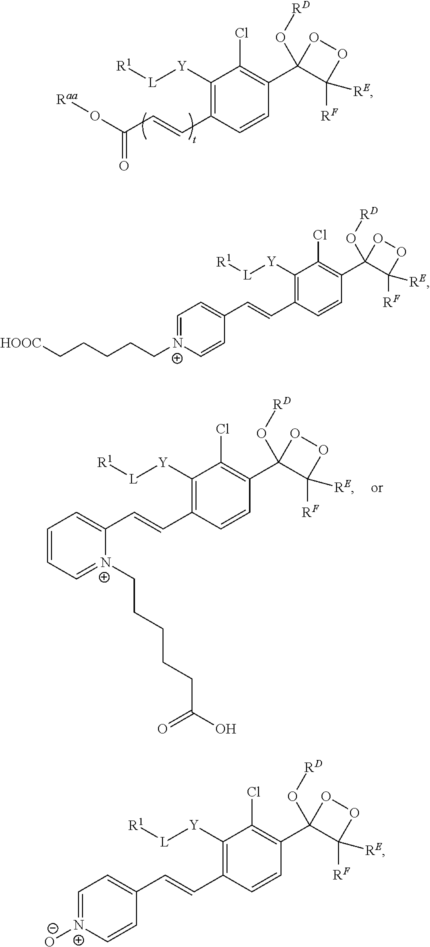

preferably

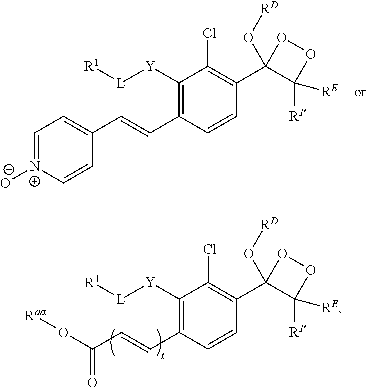

and the compound of Formula Ib has the structure

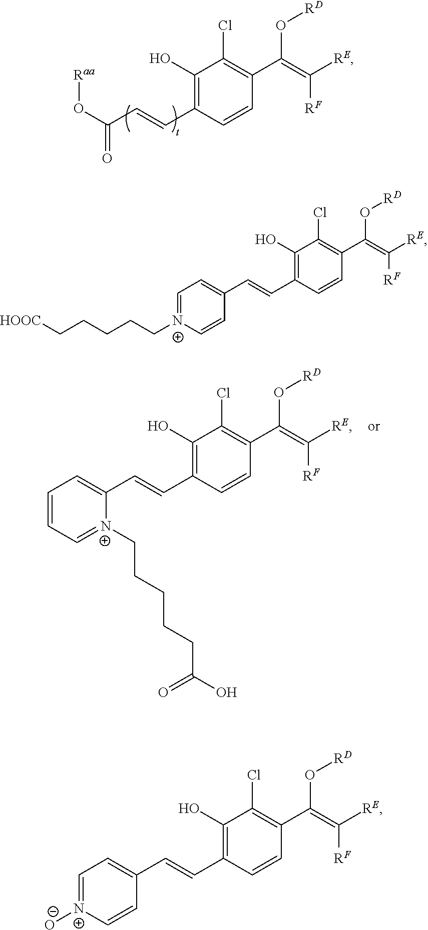

preferably

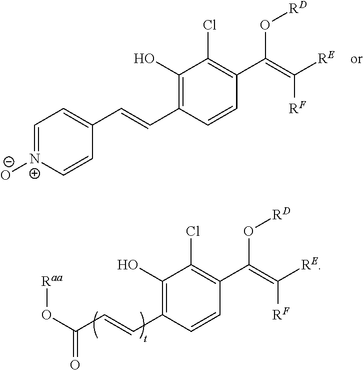

According to another particularly preferred embodiment, the compound of Formula Ia has the structure

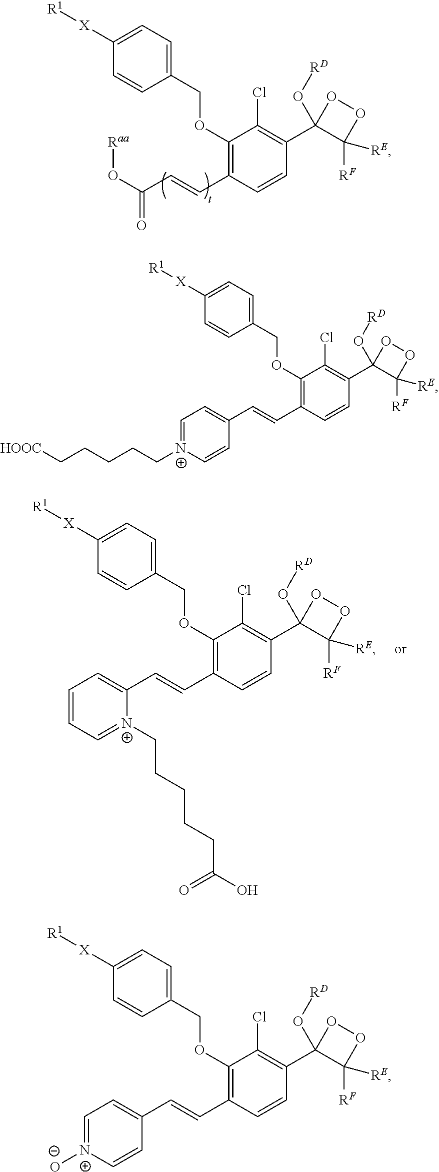

preferably

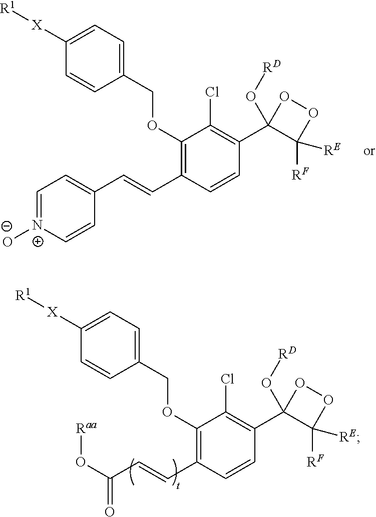

and the compound of Formula Ib has the structure

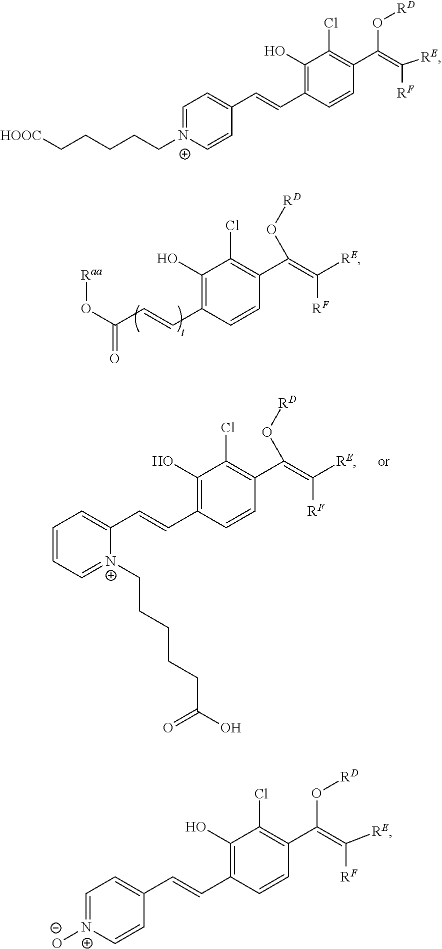

preferably

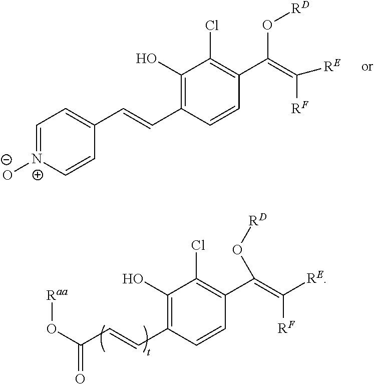

Accordingly to another particularly preferred embodiment, the compound of Formula Ia has the structure

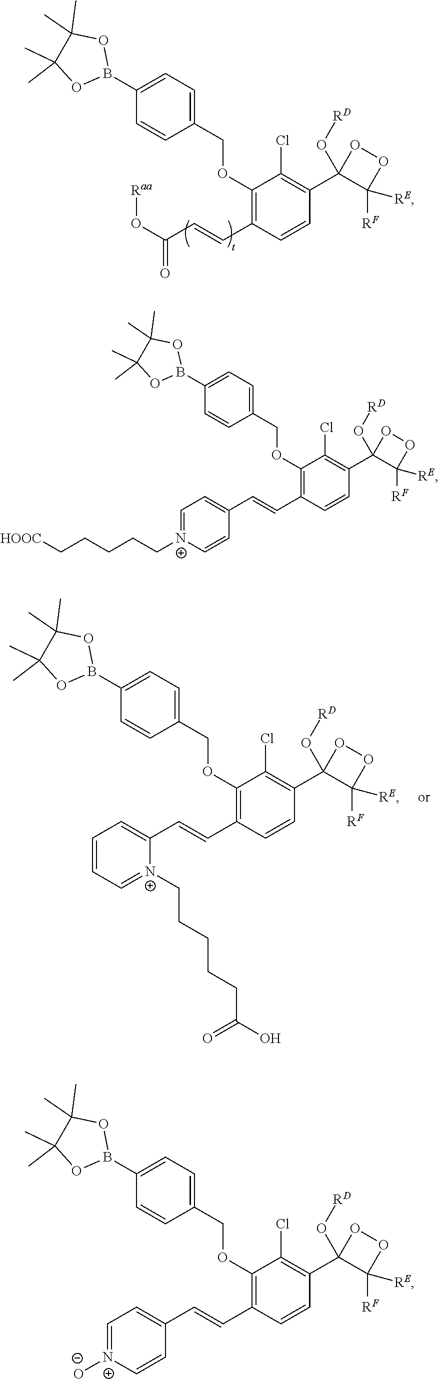

preferably

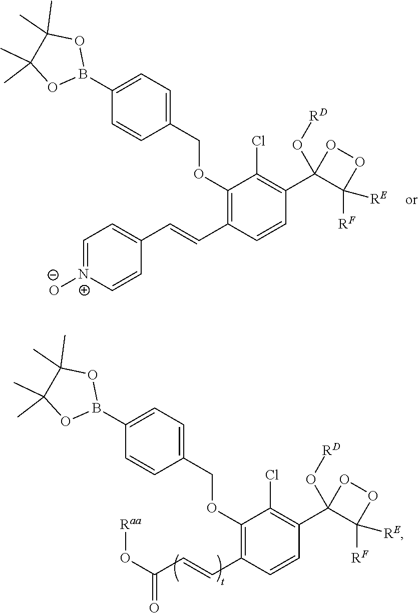

and the compound of Formula Ib has the structure

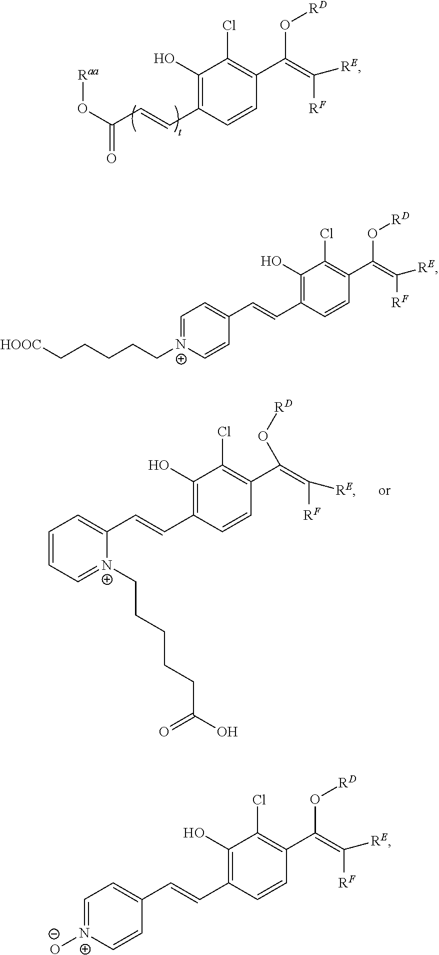

preferably

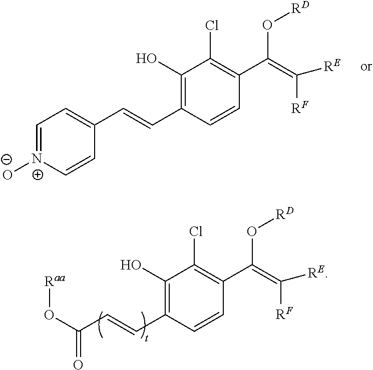

Accordingly to another particularly preferred embodiment, the compound of Formula Ia has the structure

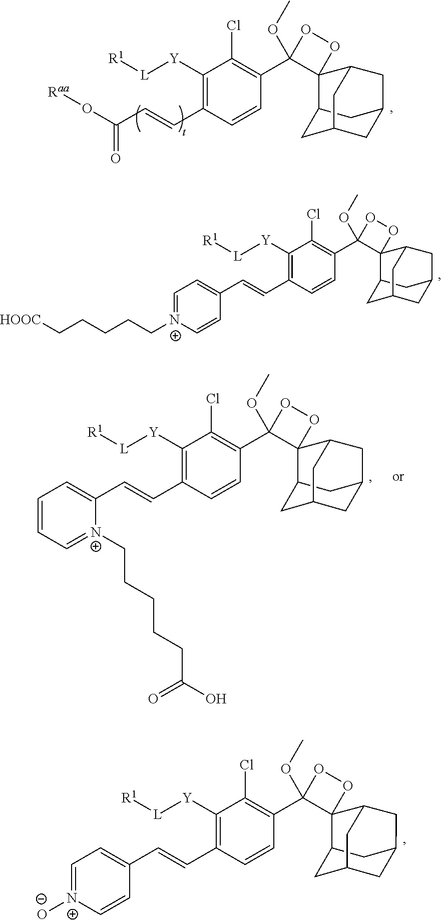

preferably

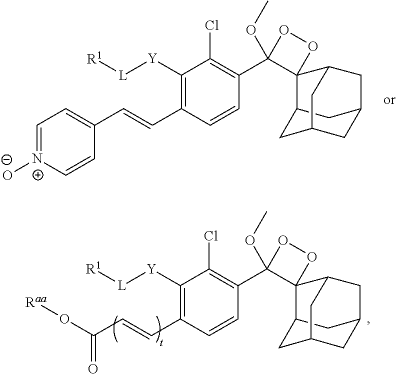

wherein the adamantyl moiety is optionally substituted;
and the compound of Formula Ib has the structure

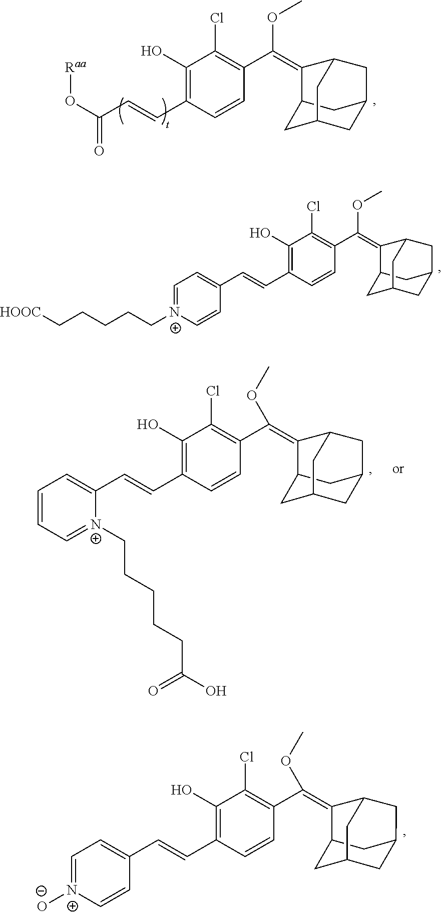

preferably

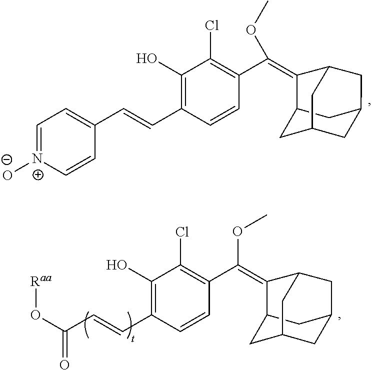

wherein the adamantyl moiety is optionally substituted.

Accordingly to another particularly preferred embodiment, the compound of Formula Ia has the structure

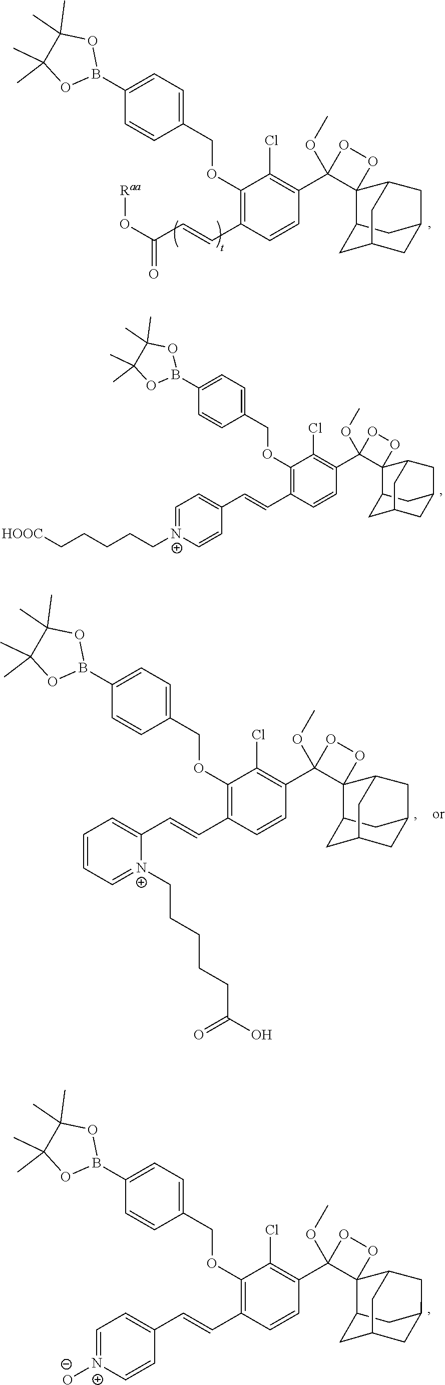

preferably

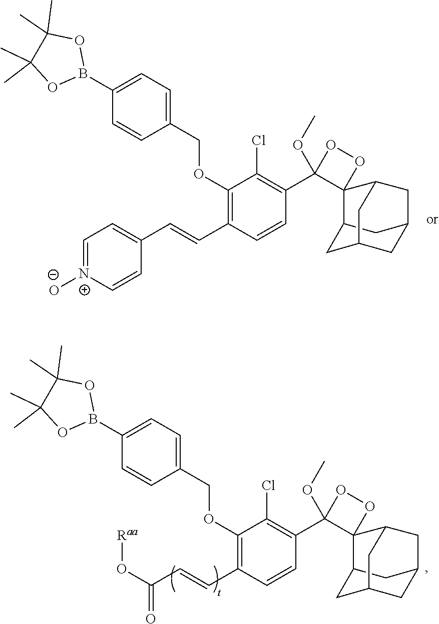

wherein the adamantyl moiety is optionally substituted; and the compound of Formula Ib has the structure

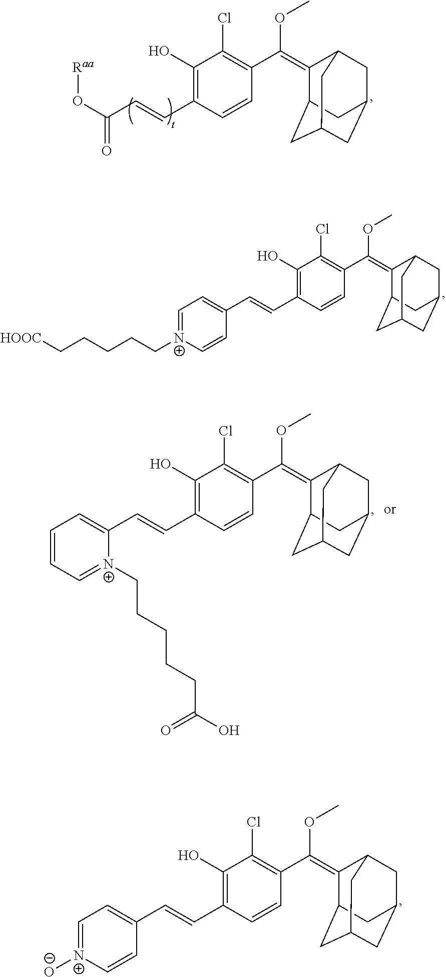

preferably

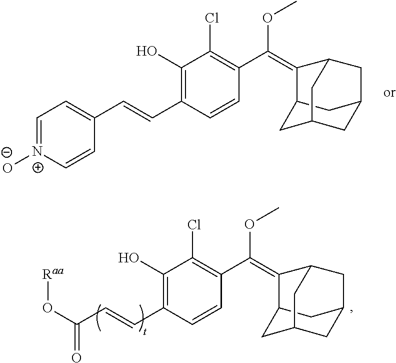

wherein the adamantyl moiety is optionally substituted.

In a second aspect, the present invention relates to a compound of Formula II

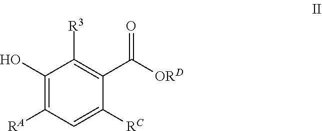

Substituents R³, R^{A}, R^{C}, and R^{D} are as defined in the first aspect, including all preferred embodiments thereof.

In a particular preferred embodiment of a compound of Formula II, R³ is —Cl. It is also preferred that R^{C} is H and R^{A} is R². It is also preferred that R^{D} is methyl. In a particular preferred embodiment, a compound of Formula II has the following structure:

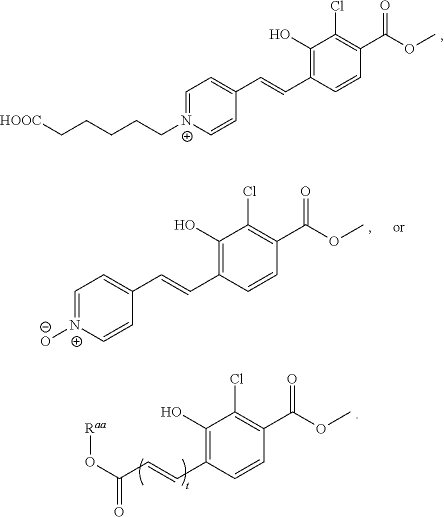

As set out above, upon reaction with an analyte and luminescence triggered thereby, compounds of Formula Ia and Ib are ultimately converted into a compound of Formula II.

Although a compound of Formula II formed from a compound of Formula Ia or Ib by means of reaction with an analyte and subsequent chemiluminescence is not chemiluminescent (anymore), said compound has still valuable properties, in particular it is a very good fluorescent dye with an emission maximum in the long wavelength range, depending on substituent R².

Hence, one particular preferred and useful application of a compound of Formula Ia or Ib is to bind it (preferably covalently) to a biomolecule, e.g., an antibody, a nucleic acid, or a protein, where it can on the one hand be used a chemiluminescent analyte-specific label and, on the other hand, after reaction with an analyte and chemiluminescence, it can still be used as fluorescent label.

In a third aspect, the present invention relates to a composition comprising a compound of Formula Ia or Ib and a carrier.

The carrier is preferably a pharmaceutically acceptable carrier.

In a fourth aspect, the present invention relates to a ready-for-use injectable solution comprising a compound of Formula Ia or Ib.

In a fifth aspect, the present invention relates to a compound of Formula Ia or Ib, a composition comprising a compound of Formula Ia or Ib and a carrier, or a ready-for-use injectable solution comprising a compound of Formula Ia or Ib for use in in vivo diagnostics or imaging.

In particular, it has been shown that the compounds of Formulae Ia and Ib are particularly suitable for imaging/detecting inflammatory processes and tumors. For example, if R¹ is —B(Z″)₃⁻Kat⁺ or —B(Z)(Z′) including the preferred embodiments thereof set out above, the compound of Formula Ia is useful for visualizing/detecting the presence/overexpression of peroxides. If R¹ is selected from the group consisting

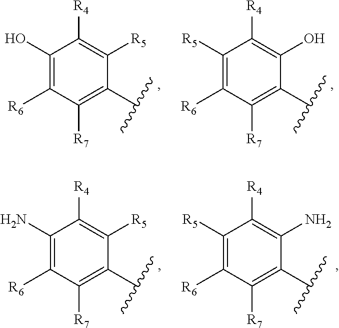

including the preferred embodiments thereof set out above, the compound of Formula Ia is useful for visualizing/detecting the presence/overexpression of reactive oxygen species (ROS or ROX) and reactive nitrogen species (RNS or RNX). If the compound of Formula Ia or Ib is a compound of Formula Ib, said compound is suitable for visualizing/detecting the presence/overexpression of singlet oxygen. If R¹ is selected from —NO₂, or azide, the compound of Formula Ia is useful for visualizing/detecting reductases, e.g. nitroreductase or cytochrome P450, which is able to reduce an azide group in an oxygen-dependent manner, which may be used for detecting hypoxia. If R¹ is responsive towards a peptidase, the compound of Formula Ia is useful for visualizing/detecting the overexpression of peptidases (e.g. cathepsin).

In a sixth aspect, the present invention relates to the use of a compound of Formula Ia or Ib for in vitro imaging.

It has been shown that the compound is not only highly advantageous for in vivo imaging, but also shows particularly good properties for in vitro imaging.

In a seventh aspect, the present invention relates to the use of a compound of Formula Ia or Ib in an in vitro assay for the detection of singlet oxygen.

In a eights aspect, the present invention relates to the use of a compound of Formula Ia in any in vitro assay for the detection of a peroxide or an enzyme.

Preferably, the peroxide is hydrogen peroxide, a reactive oxygen species, or a reactive nitrogen species. Exemplary enzymes and respective groups R¹ are set out in the first aspect. For example, the enzyme may be a reductase, e.g. a nitroreductase or cytochrome P450, and R¹ is —NO₂, or azide, or the enzyme may be a peptidase (e.g. cathepsin) and R¹ is responsive towars a reductase. Exemplary groups R¹ that are responsive towards reductases are shown in the first aspect.

In a ninth aspect, the present invention relates to a method for determining the presence, or measuring the level, of an analyte in a sample.

the method comprises the following steps:

(a) contacting the sample with a compound of Formula Ia or Ib thereby converting said compound into an emissive species; and
(b) detecting the emission of said emissive species.

Preferably, the analyte is an enzyme and R¹ is a group responsive towards/cleavable by said enzyme.

In one preferred embodiment, the analyte is hydrogen peroxide and R¹ is —B(Z″)₃⁻Kat⁺ or —B(Z)(Z′), preferably —B(Z)(Z′), more preferably —B(OH)₂ or

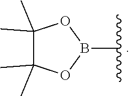 [SMILES: *B1OC(C)(C)C(C)(C)O1]

In another preferred embodiment, the analyte is singlet oxygen and the compound is a compound of Formula Ia. In another preferred embodiment, the analyte is a reactive oxygen species or a reactive nitrogen species and R¹ is selected from the group consisting of

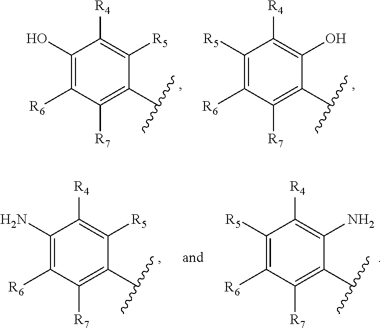

In another preferred embodiment, the analyte is a reductase, e.g. a nitroreductase or cytochrome P450, and R¹ is —NO₂, or azide. In another preferred embodiment, the analyte is a peptidase and R¹ is selected from the group consisting of

| | | |
|---|---|---|
| 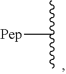 | amino acid having the formula | di-peptidyl having the formula |
| | 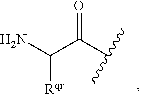 | 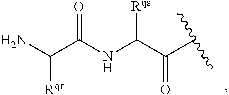 |
| | wherein R^{qr} is a side | wherein R^{qr} and R^{qs} |
| | group depending on the | are side groups |
| | respective amino acid, | depending on the |
| | wherein said amino acidyl | respective amino acids |
| | is preferably selected | of which the di-peptidyl |
| | from L-alaninyl, L- | group is composed of, |
| | leucinyl, and β-alanyl, | wherein X is preferably |
| | and wherein X is | —NH— if L is present, |
| | preferably —NH— if L is | |
| | present, | |
| | SucOMe-Arg-Pro- | L-alanyl (A-), |
| tri-peptidyl having the formula | Tyrosinyl (SucOMe-RPY-), | |
| 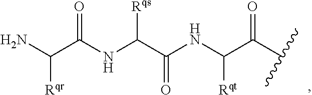 | | |
| wherein R^{qr}, R^{qs}, and R^{qt} are | | |
| side groups depending on the | | |
| respective amino acids of | | |
| which the tri-peptidyl group is | | |
| composed of, wherein X is | | |
| preferably —NH— if L is | | |
| present, | | |
| L-leucinyl (L-), and | β-alanyl, | |

In another preferred embodiment, the analyte is LacZ and R¹ is beta-D-galactopyranoside.

Preferably, the sample is a biological sample. Preferably, the biological sample is a bodily fluid, a bodily fluid-based solution, or a tissue biopsy sample. Preferably, the method of the ninth aspect is an in vitro method.

In a tenth aspect, the present invention relates to the use of a compound of Formula Ia or Ib as a label for a biomolecule, preferably an antibody, a nucleic acid, or a protein.

In an eleventh aspect, the present invention relates to a biomolecule, preferably an antibody, a nucleic acid, or a protein, characterized in that it is bound to a compound of Formula Ia or Ib as a label.

Or in other words, the present invention also relates to a labelled biomolecule, wherein the label is a compound of Formula Ia or lb.

Preferably, the compound of Formula Ia or Ib is covalently bound to the biomolecule.

A biomolecule labelled with a compound of Formula Ia or Ib may be used, e.g., in immunohistochemical applications in cancer diagnosis.

In an eleventh aspect, the present invention relates to a biomolecule of the eleventh aspect, preferably an antibody, for use in cancer diagnosis.

The present invention is now described in more detail by means of items 1 to 65:

Item 1:

A compound of Formula Ia or Ib

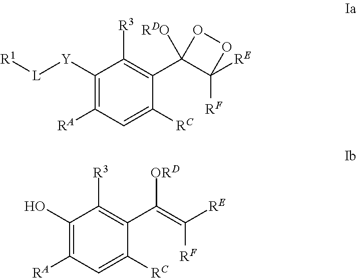

wherein
R^{D} is selected from a linear or branched C1-C18 alkyl or C3-C7 cycloalkyl, preferably R^{D} is methyl or ethyl, more preferably methyl;
R^{E} and R^{F} are independently selected from a branched C3-C18 alkyl or C3-C7 cycloalkyl, or R^{E} and R^{F} together with the carbon atom to which they are attached form an optionally substituted fused, spiro or bridged cyclic or polycyclic ring, preferably adamantyl, wherein the adamantyl may be substituted;
R³ is —H, —F, —Cl, —Br, —I, —CF₃, —NO₂, —CN, —COOR^{XX}, —C(O)R^{XX}, —SO₂R^{XX} or R², preferably R³ is —Cl;
R^{A} and R^{D} are independently selected from —H, —F, —Cl, —Br, —I, —CF₃, —NO₂, —CN, —R^{x}COOR^{XX}, —COOR^{XX}, —C(O)R^{XX}, —SO₂R^{XX} and R²;
R^{x} is linear or branched C1-C6 alkylene or linear or branched C1-C6 alkenylene, preferably —CH═CH—;
R^{XX} is linear or branched C1-18 alkyl, C2 to C8 alkenyl or C2 to C8 alkynyl chain or —H; provided that at least one, preferably one, of R³, R^{A} and R^{C} is R², and more preferably that R³ is as defined above and R^{A} is R² and R^{C} is H, or that R³ is as defined above and R^{A} is H and R^{C} is R²;
R² is selected from the group consisting of

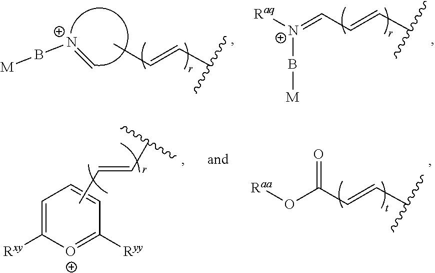

wherein

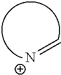

denotes a mono- or polycyclic, aromatic or nonaromatic ring system comprising the moiety

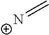

as a ring member,
wherein the moiety

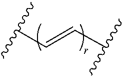

is connected to

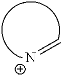

via an atom, which is a member of said mono- or polycyclic, aromatic or nonaromatic ring system, provided that a delocalized π-system extends from the positively charged nitrogen atom of

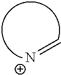

via moiety

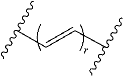

to the central aromatic ring of the compound of Formula Ia or Ib,
wherein each ring of said mono- or polycyclic, aromatic or nonaromatic ring system may be substituted with one or more groups selected from —OH, —CN, —SO₃⁻, linear or branched C1-C6 alkyl, linear or branched C2-C6 alkenyl, linear or branched C2-C6 alkynyl, a polyethylene glycol chain or a polypropylene glycol chain,
wherein

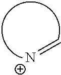

is optionally substituted with one or two negatively charged substituent(s), preferably selected from −COO⁻ and —SO₃⁻, in ortho position to the positively charged nitrogen atom,
r is selected from the group consisting of 1, 2, 3, 4, 5, and 6, preferably r is 1,
R^{xy} and R^{yy} are independently selected from H, linear or branched C1-C6 alkyl, linear or branched C2-C6 alkenyl, linear or branched C2-C6 alkynyl, and C3-C7 cycloalkyl groups, preferably R^{xy} and R^{yy} are independently selected from methyl, ethyl, propyl, isopropyl, butyl, sec-butyl, and tert-butyl,
R^{aq} is a linear or branched C1 to C8 alkyl, preferably C2 to C6 alkyl, a linear or branched C2 to C8 alkenyl, a linear or branched C2 to C8 alkynyl, or a linear or branched C4 to C12 heteroalkyl, wherein the linear or branched C1 to C8 alkyl, C2 to C8 alkenyl, C2 to C8 alkynyl, or C4 to C12 heteroalkyl may be substituted with one or more groups selected from —OH, —COOH, halogen, preferably —Cl or —F, and —NH₂ and wherein the linear or branched C1 to C8 alkyl, C2 to C8 alkenyl or C2 to C8 alkynyl chain may comprise one or more —O— or —CO— groups within the chain.
M is an optionally present group,
wherein, if M is absent, B is —O^{⊖}, H, a linear or branched C1 to C8 alkyl, preferably a linear or branched C2 to C6 alkyl, a linear or branched C2 to C8 alkenyl or a linear or branched C2 to C8 alkynyl chain,
wherein the linear or branched C1 to C8 alkyl, C2 to C8 alkenyl or C2 to C8 alkynyl chain may be substituted with one or more groups selected from —OH, —COOH, halogen, preferably —Cl or —F, —NH₂ and a group capable of binding to a functional group of a peptide, endolysine, or protein, wherein said functional group of a peptide, endolysine, or protein is selected from an amino, carboxy, or mercapto group, thus allowing for binding said peptide, endolysine, or protein to B; and
wherein the linear or branched C1 to C8 alkyl, C2 to C8 alkenyl or C2 to C8 alkynyl chain may comprise one or more —O— or —CO— groups within the chain,
preferably B is —O^{⊖}, H, —CH₃, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —CH═CH₂, —CH═CHCH₃, —CH₂CH═CH₃, or a linear or branched C4-C8 alkenyl group,
preferably, if M is absent and B is H,

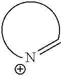

is substituted with one or two, preferably two, —COO⁻ groups in ortho position to the positively charged nitrogen atom,
or wherein, if M is present, B is a linear or branched C1 to C8 alkylene, preferably C2 to C6 alkylene, a linear or branched C2 to C8 alkenylene or linear or branched C2 to C8 alkynylene chain,
wherein the linear or branched C1 to C8 alkylene, C2 to C8 alkenylene or C2 to C8 alkynylene chain may be substituted with one or more groups selected from —OH, —COOH, halogen, preferably —Cl or —F, —NH₂ and a group capable of binding to a functional group of a peptide, endolysine, or protein, wherein said functional group of a peptide, endolysine, or protein is selected from an amino, carboxy, or mercapto group, thus allowing for binding said peptide, endolysine, or protein to B; and
wherein the linear or branched C1 to C8 alkylene, C2 to C8 alkenylene or C2 to C8 alkynylene chain may comprise one or more —O— or —CO— groups within the chain, preferably B is —CH₂—, —(CH₂)₂—, —(CH₂)₃—, —(CH₂)₄—, —(CH₂)₅—, —(CH₂)₆—, —(CH₂)₇—, —(CH₂)₈—, —CH═CH—, —CH₂CH═CHCH₂—, a linear or branched C6 alkenylene group with one or two double bonds or a linear or branched C8 alkenylene group with one, two or three double bonds, and
M is selected from the group consisting of cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, tetrazole, optionally substituted aryl, optionally substituted alkenyl,

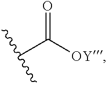

carbonyl having the structure

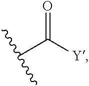

amide, an amide having the structure

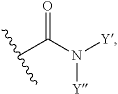

or M is a moiety including one or more groups selected from cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, and tetrazole, optionally substituted aryl, optionally substituted alkenyl,

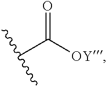

carbonyl having the structure

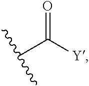

amide, an amide having the structure

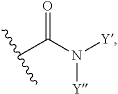

wherein Y′″ is —H, an optionally substituted C1-C8 alkyl, optionally substituted C2-C8 alkenyl, optionally substituted C2-C8 alkynyl, an alkali metal ion or a negative charge, wherein Y′ and Y″ are independently selected from —H, an optionally substituted C1-C8 alkyl, optionally substituted C2-C8 alkenyl, or an optionally substituted C2-C8 alkynyl, or Y′ and Y″ together with the nitrogen atom to which they are attached form an optionally substituted heterocyclic structure, preferably an optionally substituted maleimide group;
preferably M is —COOH, —SO₃⁻, a moiety derived from an amino acid, a moiety derived from a monosaccharide or a disaccharide, a moiety derived from a polycarboxylic acid, a moiety derived from polyethylene glycol or polypropylene glycol, or a moiety derived from a polyol;
more preferably M is —COOH or —SO₃⁻,
t is 2, 3, or 4;
R^{aa} is —H, a linear or branched C1-6 alkyl, preferably methyl or ethyl, more preferably methyl, a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group such as

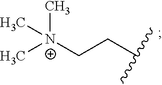 [SMILES: *CC[N+](C)(C)C]

Y is absent or is —O—, provided that Y is absent if R¹ is —B(Z)(Z′) or —B(Z″)3⁻Kat⁺ and L is absent,
wherein Z and Z′ are independently selected from R^{ab} and OR^{ac},
wherein R^{ab} is selected from the group consisting of —OH, —O⁻Kat⁺, optionally substituted C1-C4 alkyl, optionally substituted C2-C4 heteroalkyl, optionally substituted C2-C4 alkenyl, optionally substituted C2-C4 heteroalkenyl, optionally substituted C2-C4 alkynyl, optionally substituted C2-C4 heteroalkynyl, optionally substituted C5-C6 aryl, optionally substituted C5-C6 heteroaryl, optionally substituted C6-C10 aralykl, and optionally substituted C6-C10 heteroaralkyl,
and R^{ac} is selected from the group consisting of —H, optionally substituted C1-C4 alkyl, optionally substituted C2-C4 heteroalkyl, optionally substituted C2-C4 alkenyl, optionally substituted C2-C4 heteroalkenyl, optionally substituted C2-C4 alkynyl, optionally substituted C2-C4 heteroalkynyl, optionally substituted C5-C6 aryl, optionally substituted C5-C6 heteroaryl, optionally substituted C6-C10 aralykl, and optionally substituted C6-C10 heteroaralkyl, or
wherein two R^{ab}, two R^{ac} or one R^{ab} and one R^{ac} together with their intervening atoms form a 5- to 7-membered optionally substituted heterocyclic ring, preferably a saturated optionally substituted heterocyclic ring;
Z″ is selected from —F, —Cl, —Br, —I, preferably Z″ is —F;
Kat⁺ is an organic or inorganic cation, preferably an alkali metal cation;
L is absent or is a linker selected from the group consisting of moieties L1 to L8

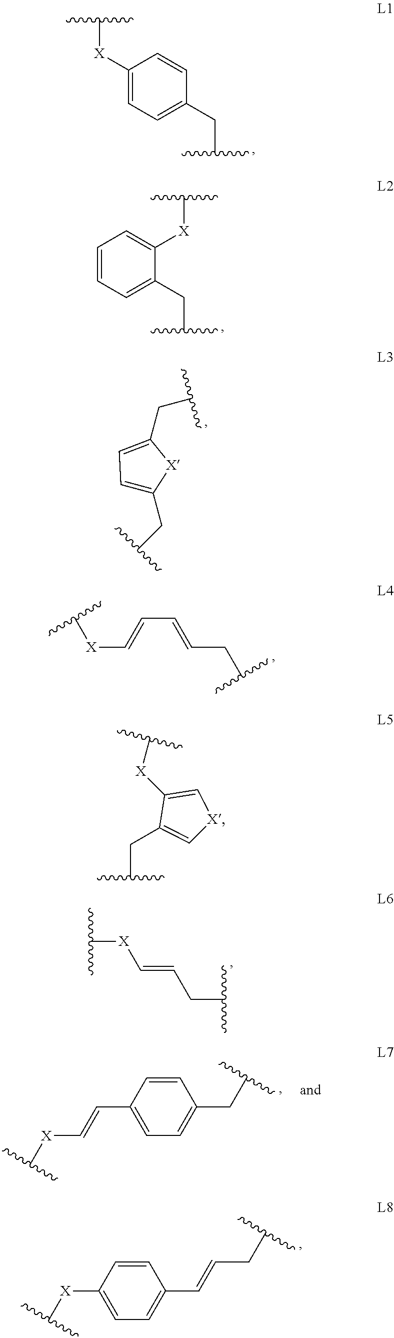

wherein
X is absent or is —O—, —NH—, —NR^{G}—, —S—, or —NH—COO— wherein the COO-moiety is bound to R¹, wherein R^{G} is selected from a substituted or unsubstituted C1-C12 alkyl, preferably
X is absent or is —O— or —NH—, provided that X is absent if R¹ is —B(Z)(Z′), —B(Z″)3⁻Kat⁺, —NO₂ or an azide group,
X′ is selected from —S—, —O—, —NH—, and —NR^{G}—, wherein R^{G} is selected from a substituted or unsubstituted C1-C12 alkyl,
X is connected to R¹,
wherein each of L1 to L8 is optionally functionalized with a group capable of binding to a functional group of a peptide, endolysine, or protein, or a cell membrane-permeable group, wherein said functional group of a peptide, endolysine, or protein is selected from an amino, carboxy, or mercapto group, thus allowing for binding said peptide, endolysine, or protein to L,
provided that L is absent and R¹ is —B(Z)(Z′), —B(Z″)₃⁻Kat⁺ if Y is absent, and provided that Y is —O— if L is present,
R¹ is an analyte-responsive group capable of reacting with an analyte, wherein
if L is present and X is present, then X—R¹ is converted into a XH group upon reaction of R¹ with said analyte, or
if L is present and X is absent, then R¹ is converted into a π-donor group upon reaction of R¹ with said analyte, or
if L and Y are absent and R¹ is —B(Z)(Z′) or —B(Z″)₃⁻Kat⁺, then R¹ is converted into a —OH group, or
if L is absent and Y is —O—, then the —O—R¹ moiety is converted into a —OH group.

Item 2: The compound according to item 1, wherein

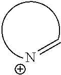

denotes a mono-, bi- or tricyclic, aromatic or nonaromatic ring system comprising the moiety

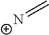

as a ring member.

Item 3: The compound according to item 2, wherein

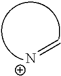

denotes a monocyclic aromatic ring comprising the moiety

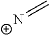

as a ring member.

Item 4: The compound according to item 2, wherein

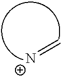

denotes a bicyclic aromatic ring comprising the moiety

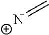

as a ring member.

Item 5: The compound according to item 2, wherein

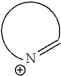

denotes a tricyclic aromatic ring comprising the moiety

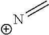

as a ring member.

Item 6: The compound according to any one of the preceding items, wherein R² is selected from the group consisting of

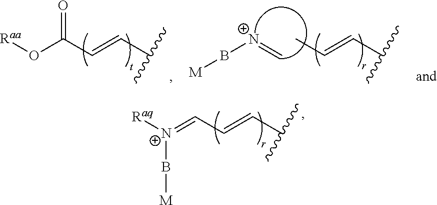

preferably R² is

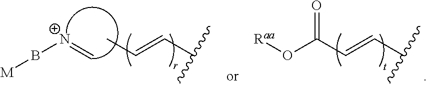

Item 7: The compound according to any one of the preceding items, wherein R² is selected from the group consisting of

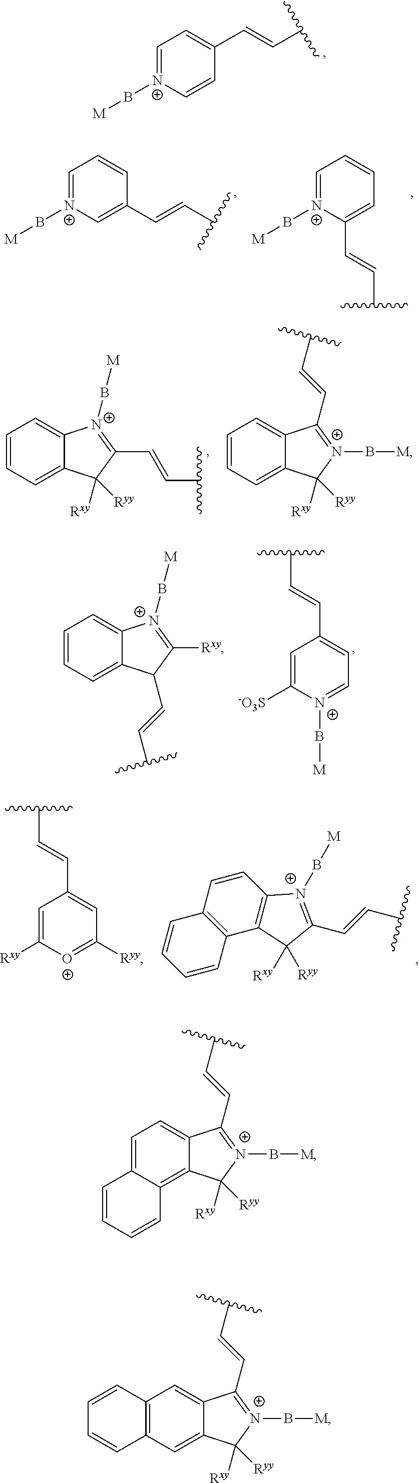

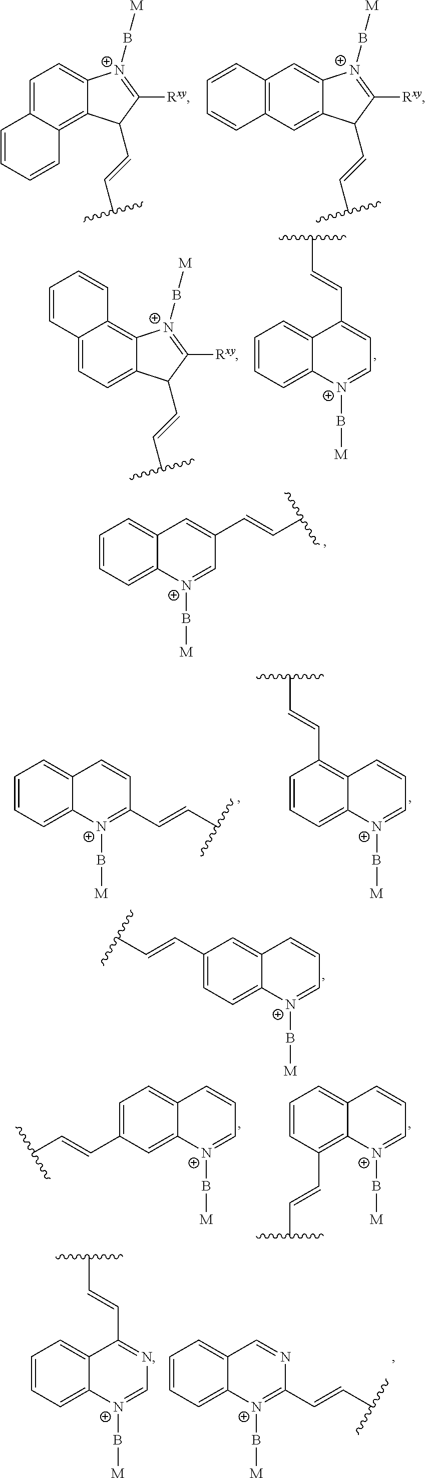

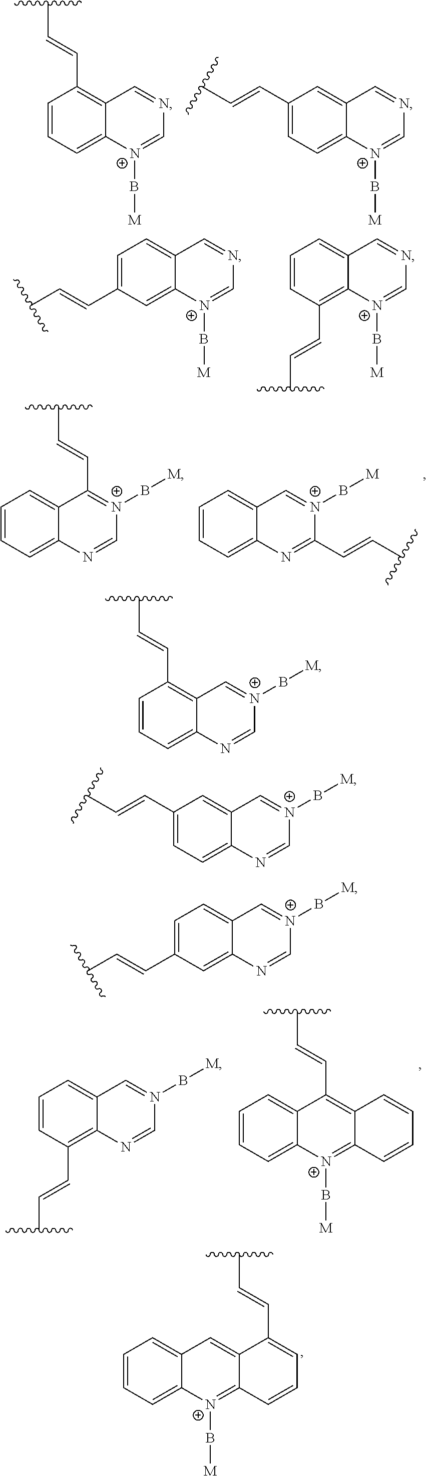

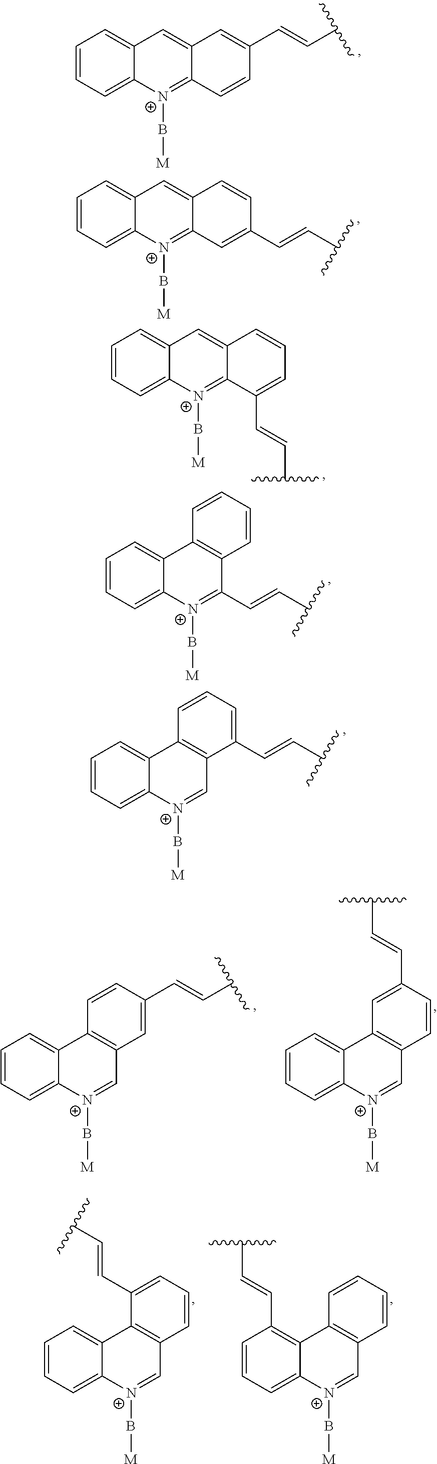

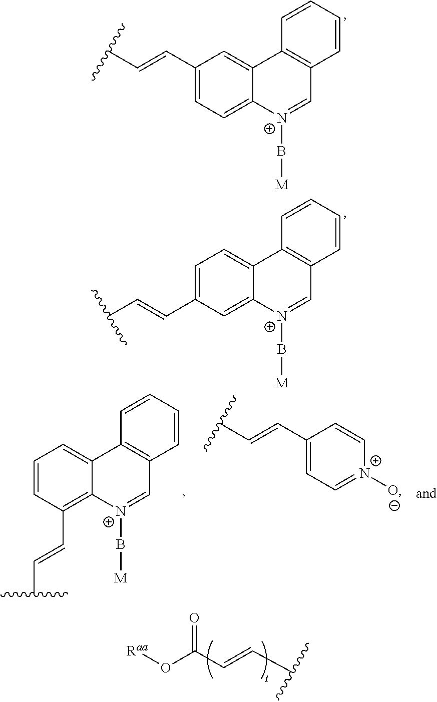

wherein the aromatic ring(s) of R² may be substituted with one or more groups selected from —OH, —CN, —SO₃⁻, linear or branched C1-C6 alkyl, linear or branched C2-C6 alkenyl, and linear or branched C2-C6 alkynyl, a polyethylene glycol chain or a polypropylene glycol chain,
wherein, if the respective position is available for substitution, the aromatic ring is optionally substituted with one or two negatively charged substituent(s), preferably selected from −COO⁻ and —SO₃⁻, in ortho position to the positively charged nitrogen atom,
and R^{xy}, R^{yy}, M and B are as defined in item 1.

Item 8: The compound according to any one of the preceding items, wherein R² is selected from the group consisting of

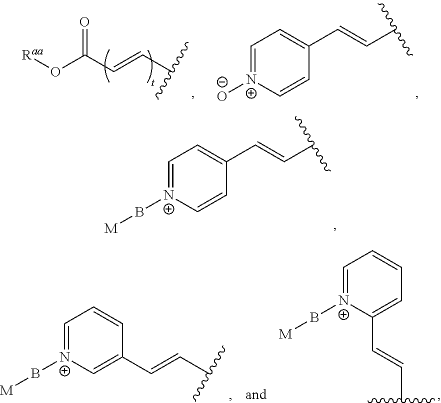

wherein M and B are as defined before, preferably from the group consisting of

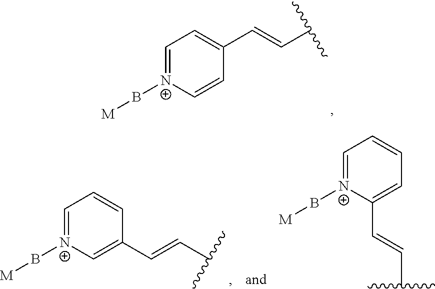

wherein, if the respective position is available for substitution, the aromatic ring is optionally substituted with one or two negatively charged substituent(s), preferably selected from −COO⁻ and —SO₃⁻, in ortho position to the positively charged nitrogen atom.

Item 9: The compound according to any one of the preceding items, wherein R² is

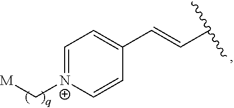

wherein q is 2, 3, 4, 5, 6, 7, 8, 9, 10, or 11, preferably 3, 4, 5, 6, or 7, more preferably 5, or wherein R² is

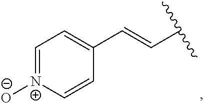 [SMILES: */C=C/c1cc[n+]([O-])cc1]

or wherein R² is

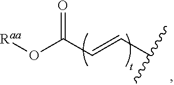

preferably wherein R² is

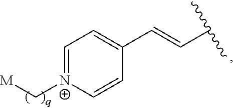

wherein q is 2, 3, 4, 5, 6, 7, 8, 9, 10, or 11, preferably 3, 4, 5, 6, or 7, more preferably 5,
wherein, if the respective position is available for substitution, the aromatic ring is optionally substituted with one or two negatively charged substituent(s), preferably selected from −COO⁻ and —SO₃⁻, in ortho position to the positively charged nitrogen atom.

Item 10: The compound according to item 9, wherein R² is selected from the group consisting of

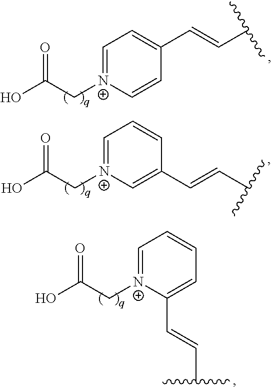

wherein q is 2, 3, 4, 5, 6, 7, 8, 9, 10, or 11, preferably 3, 4, 5, 6, or 7, more preferably 5, and

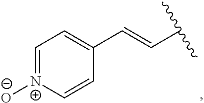

wherein, if the respective position is available for substitution, the aromatic ring is optionally substituted with one or two negatively charged substituent(s), preferably selected from −COO⁻ and —SO₃⁻, in ortho position to the positively charged nitrogen atom.

Item 11: The compound according to any one of the preceding items, wherein M is present and B is —(CH₂)_{z}—, wherein z is 1-6, preferably 3-5, more preferably, 4 or 5, even more preferably 5.

Item 12: The compound according to item 11, wherein B is —(CH₂)₁₋₆— and M is —COOH, preferably B is —(CH₂)₂₋₆— and M is —COOH, more preferably, B is —(CH₂)₅— and M is —COOH.

Item 13: The compound according to any one of items 1-8, wherein R² is

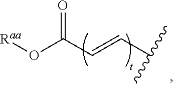

Item 14: The compound of any one of items 1-8 and 13, wherein R^{aa} is —H, a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group such as

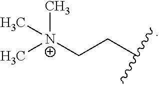 [SMILES: *CC[N+](C)(C)C]

Item 15: The compound of any one of items 1-8, 13 and 14, wherein t is greater than 2 and R^{aa} is not methyl.

Item 16: The compound according to any one of the preceding items, wherein R^{E} and R^{F} together with the carbon atom to which they are attached form a fused spiro or bridged cyclic or polycyclic ring.

Item 17: The compound according to item 16, wherein R^{E} and R^{F} together with the carbon atom to which they are attached form optionally substituted adamantyl.

Item 18: The compound according to any one of the preceding items, wherein R¹ is selected from the group shown in Table 1, wherein Pep is a group comprising a peptide moiety consisting of at least two amino acid residues and linked to L via a carboxylic acid group of said peptide moiety; provided that when R¹ is

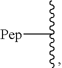

then L is present and X is —NH— or —NR^{G}—, preferably —NH—;
R₄, R₅, R₆, and R₇ are independently selected from hydrogen; C1-C6 alkyl, preferably methyl; halogen, preferably fluorine and chlorine; alkoxy, preferably methoxy; and cyano;
R₈ and R₉ are independently selected from C1-C4 alkyl, preferably methyl, or H, wherein R₈ and R₉ are preferably both methyl.

Item 19: The compound according to any one of the preceding items, wherein

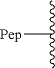

is

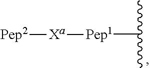

wherein
Pep¹ is a protease cleavable peptide moiety consisting of at least two amino acid residues and linked via a carboxylic group thereof to L, wherein said protease cleavable peptide moiety is optionally protected or linked through an amino group thereof to a PEG-containing group; X^{a} is absent, or is a linker linked to Pep¹ via an amide bond through either a carboxyl or amino group of Pep¹; and Pep² is absent, or a cell-penetrating peptide moiety linked to X^{a} either via an amide bond through an amino or carboxyl group thereof, or through a thiol group thereof, provided that X^{a} and Pep² are both either absent or present, and when Pep¹ is protected or linked to a PEG-containing group, X^{a} and Pep² are absent.

Item 20: The compound according to item 19, wherein Pep¹ is a peptide moiety comprising the amino acid sequence Val-Cit, Phe-Lys, Gly-Phe-Leu-Gly, Gly-Gly-Pro-Nle, Ala-Ala-Asn or His-Ser-Ser-Lys-Leu-Gln, wherein said amino acid sequence is linked via the carboxylic group of the citrulline, lysine, glycine, norleucine, asparagine or glutamine, respectively, to L; and optionally protected at an amino group thereof, or linked via an amide bond and through said amino group to a PEG-containing group.

Item 21: The compound according to item 20, wherein said PEG-containing group is of the formula

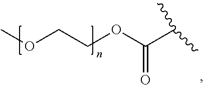

wherein n is an integer of 1 to 227.

Item 22: The compound according to item 19, wherein Pep¹ is a peptide moiety comprising the amino acid sequence Val-Cit, Phe-Lys, Gly-Phe-Leu-Gly, Gly-Gly-Pro-Nle, Ala-Ala-Asn or His-Ser-Ser-Lys-Leu-Gln, linked via the carboxylic group of the citrulline, lysine, glycine, norleucine, asparagine or glutamine, respectively, to L; X^{a} is a linker linked to Pep¹ via an amide bond through either a carboxyl or amino group of Pep′; and Pep² is a peptide moiety linked to X^{a} through a thiol group thereof.

Item 23: The compound according to item 22, wherein X^{a} is a linker of the formula

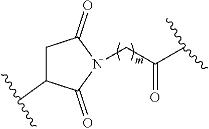

linked to Pep¹ via an amide bond through an amino group of Pep′, wherein m is an integer of 1-20, and the alkylene chain of X^{a} is optionally interrupted with one or more —O— groups; and Pep² is a peptide moiety of the sequence Cys-Gly-Lys-Arg-Lys, linked to X^{a} through the thiol group of the cysteine residue.

Item 24: The compound according to any one of the preceding items, wherein R¹ is selected from the group consisting of

| —B(Z)(Z′), | —B(Z″)₃⁻ Kat⁺, | —NO₂, |
|---|---|---|
| azide (—N₃), | 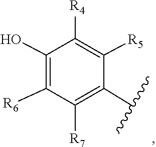 | 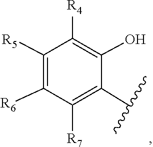 |
| 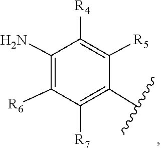 | 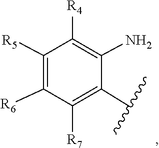 | 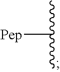 |
| amino acidyl having the | di-peptidyl having the | tri-peptidyl having the |
| formula | formula | formula |
| 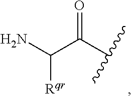 | 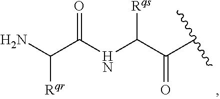 | 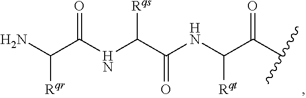 |
| wherein R^{qr} is side group | wherein R^{qr} and R^{qs} are side | wherein R^{qr}, R^{qs}, and |
| depending on the respective | groups depending on the | R^{qt} are side groups |
| amino acid, wherein said | respective amino acids of | depending on the |
| amino acidyl is preferably | which the di-peptidyl group | respective amino acids |
| selected from L-alaninyl, L- | is composed of, wherein X | of which the tri-peptidyl |
| leucinyl, and P-alanyl, and | is preferably —NH— if L is | group is composed of, |
| wherein X is preferably | present, | wherein X is preferably |
| —NH— if L is present, | | —NH— if L is present, |
| SucOMe-Arg-Pro-Tyrosinyl | L-alanyl (A-), | L-leucinyl (L-), |
| (SucOMe-RPY-), | | |
| β-alanyl, and | beta-D-galactopyranoside | |
| | 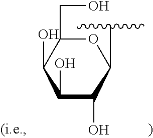 | |
| | (preferably X is —O— if L is | |
| | present, and Y is —O— if L is | |
| | absent) | |

Item 25: The compound according to any one of the preceding items, further comprising an anion balancing a positive charge on said compound, preferably a positive charge of group R², if R² comprises a positive charge, wherein said anion is preferably selected from the group consisting of a fluoride, chloride, bromide, iodide and CF₃SO₃⁻.

Item 26: The compound according to any one of the preceding items, wherein M is present.

Item 27: The compound according to any one of the preceding items, wherein R³ is —H, —F, —Cl, —Br, —I, —CF₃, or —R².

Item 28: The compound according to any one of the preceding items, wherein R^{A} and R^{C} are selected from —H, —F, —Cl, —Br, —I, —CF₃, and —R².

Item 29: The compound according to any one of the preceding items, wherein R¹ is —B(Z)(Z′) or —B(Z″)₃⁻Kat⁺, preferably —B(Z)(Z′).

Item 30: The compound according to any one of the preceding items, wherein —B(Z)(Z′) is —B(OH)₂ or

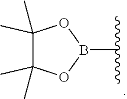 [SMILES: *B1OC(C)(C)C(C)(C)O1]

Item 31: The compound according to any one of the preceding items, wherein R¹ is —B(OH)₂ or

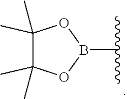 [SMILES: *B1OC(C)(C)C(C)(C)O1]

Item 32: The compound according to any one of the preceding items, wherein L is present.

Item 33: The compound according to any one of the preceding items, wherein L is

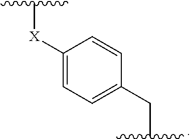

Item 34: The compound according to any one of the preceding items, wherein Y is —O—.

Item 35: The compound according to any one of the preceding items, wherein R^{A} or R^{C} is R².

Item 36: The compound according to any one of the preceding items, wherein R^{A} is R².

Item 37: The compound according to any one of the preceding items, wherein R³ is Cl, R^{A} is R², and R^{C} is H.

Item 38: The compound according to any one of the preceding items, wherein M is —COOH.

Item 39: The compound according to any one of the preceding items, wherein

R^{C} is H, and R^{A} is

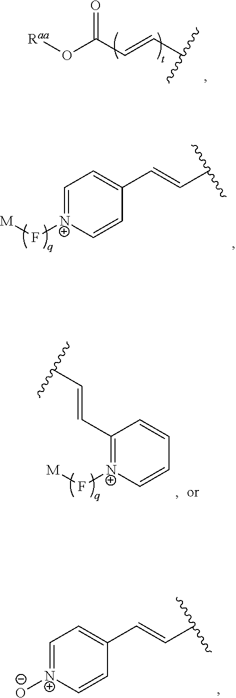

preferably

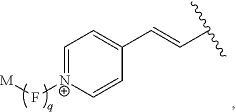

wherein F is a linear or branched C1 to C8 alkyl, preferably C2 to C6 alkyl, a linear or branched C2 to C8 alkenyl or a linear or branched C2 to C8 alkynyl chain, and wherein q is 2, 3, 4, 5, 6, 7, 8, 9, 10, or 11, preferably 3, 4, 5, 6, or 7, more preferably 5, wherein preferably R^{A} is

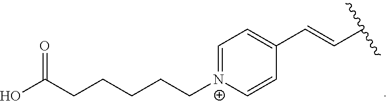 [SMILES: */C=C/c1cc[n+](CCCCCC(=O)O)cc1]

Item 40: The compound according to any one of the preceding items, wherein R^{C} is H, R^{A} is

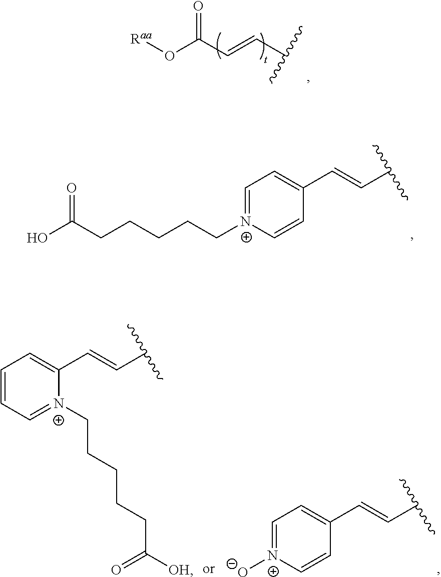

preferably

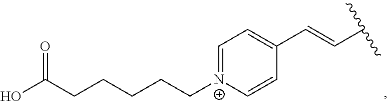 [SMILES: */C=C/c1cc[n+](CCCCCC(=O)O)cc1]

and R^{E} and R^{F} together with the carbon atom to which they are attached form optionally substituted adamantyl.

Item 41: The compound according to any one of the preceding items, wherein the compound of Formula Ia has the structure

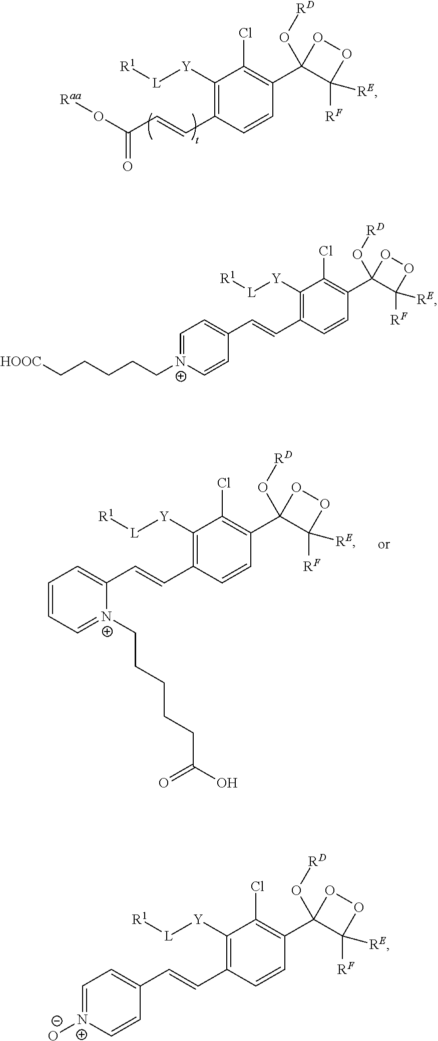

preferably

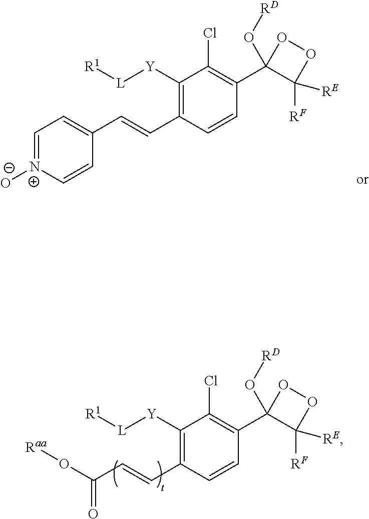

and the compound of Formula Ib has the structure

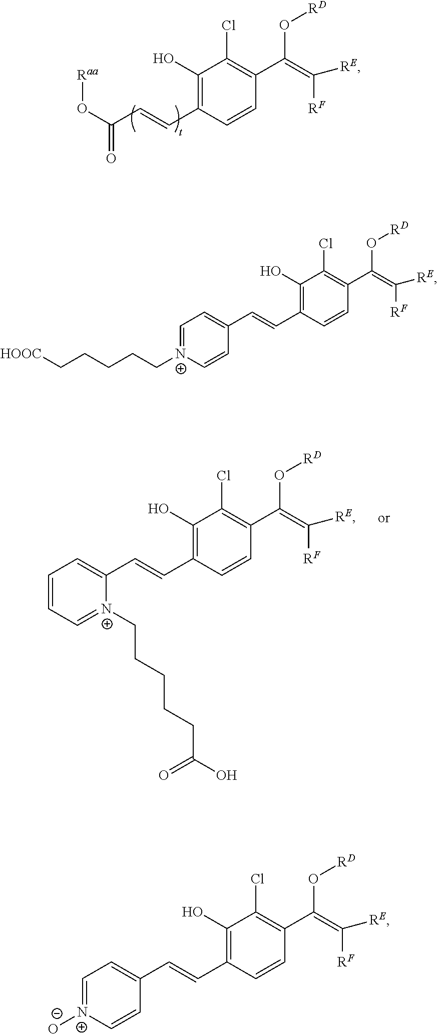

preferably

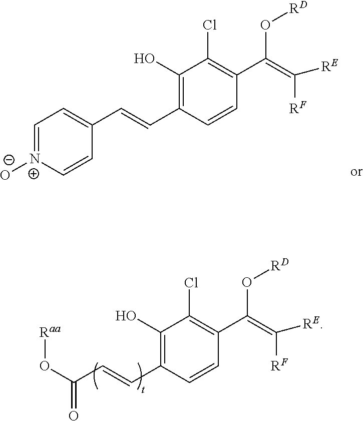

Item 42: The compound according to any one of the preceding items, wherein the compound of Formula Ia has the structure

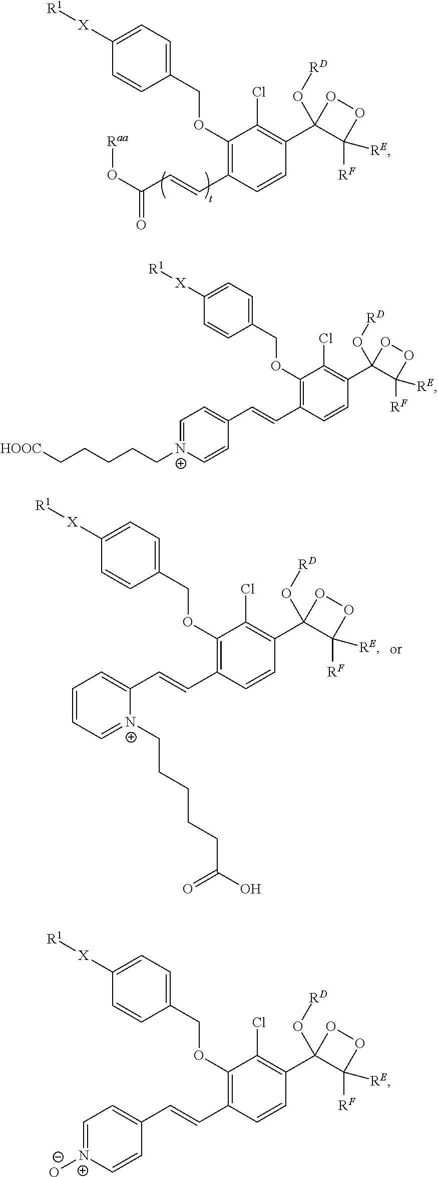

preferably

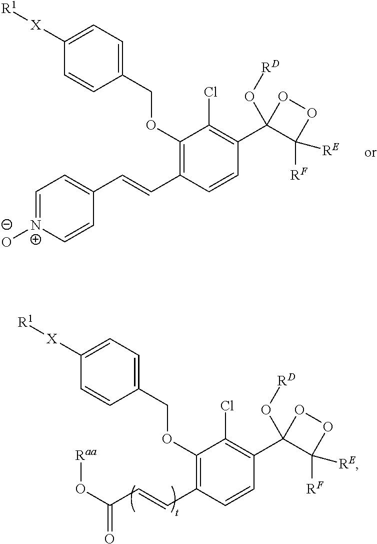

and the compound of Formula Ib has the structure

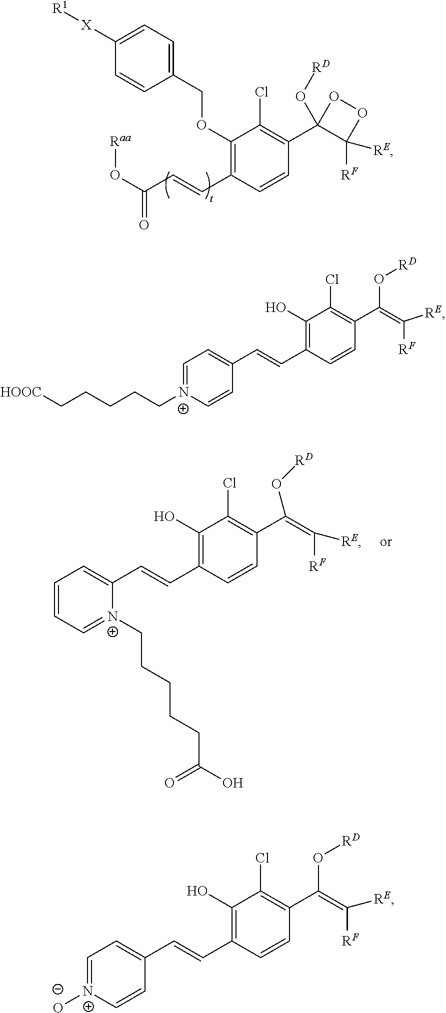

preferably

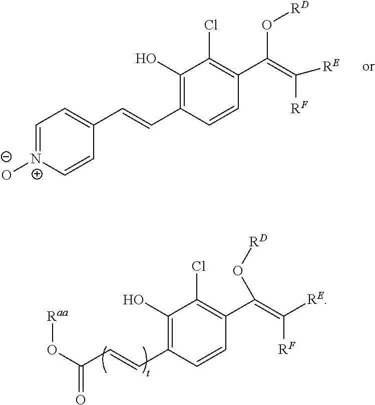

Item 43. The compound according to any one of the preceding items, wherein the compound of Formula Ia has the structure

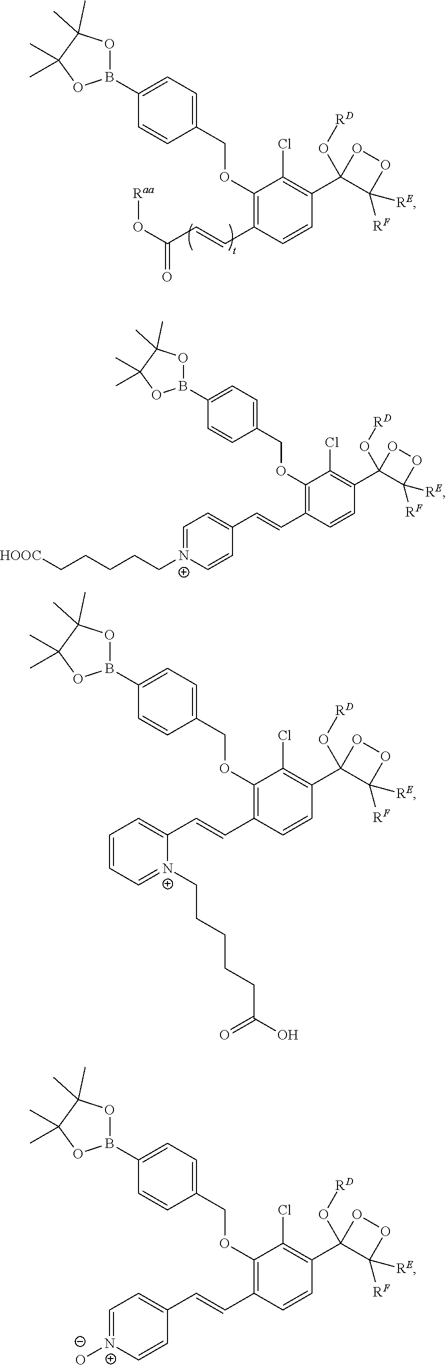

preferably

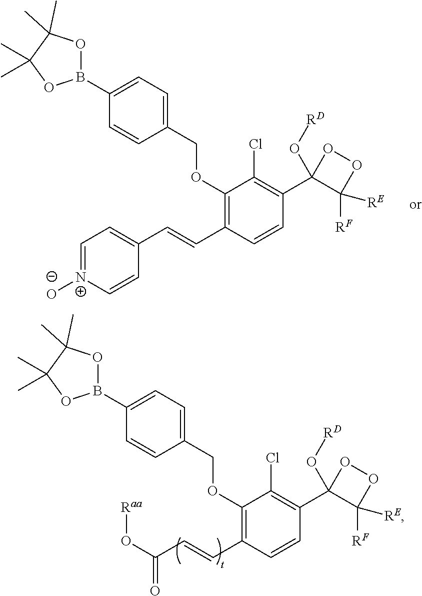

and the compound of Formula Ib has the structure

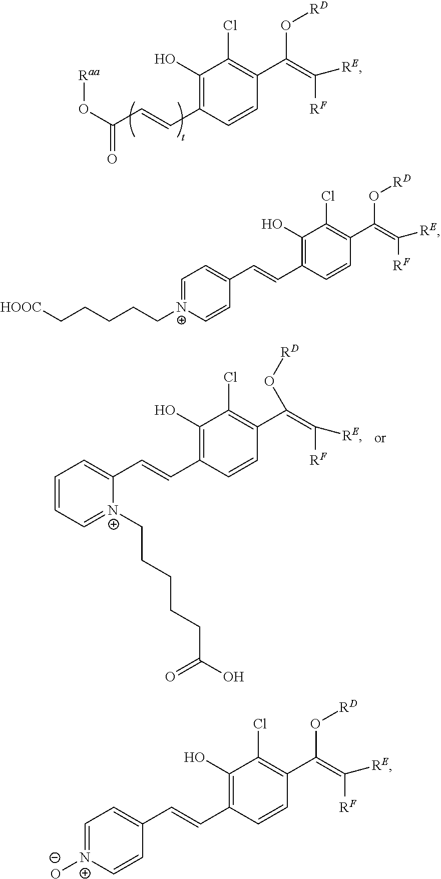

preferably

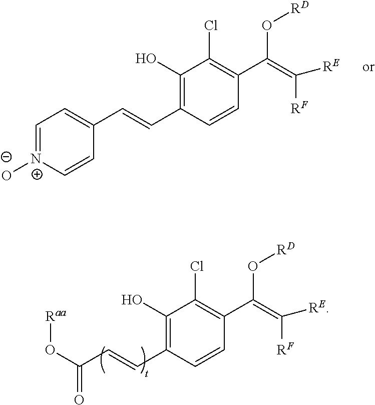

Item 44: The compound according to any one of the preceding items, wherein the compound of Formula Ia has the structure

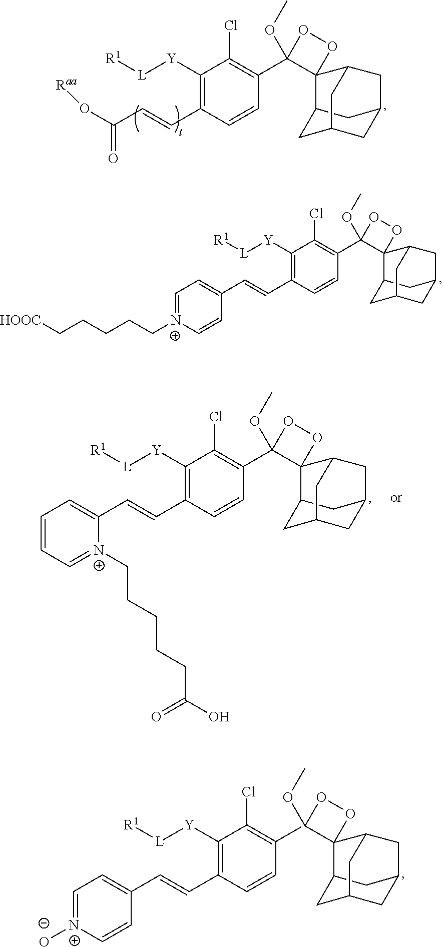

preferably

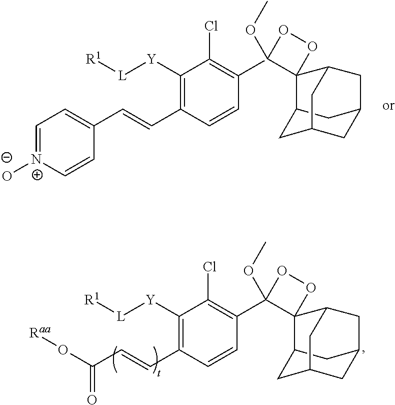

and the compound of Formula Ib has the structure

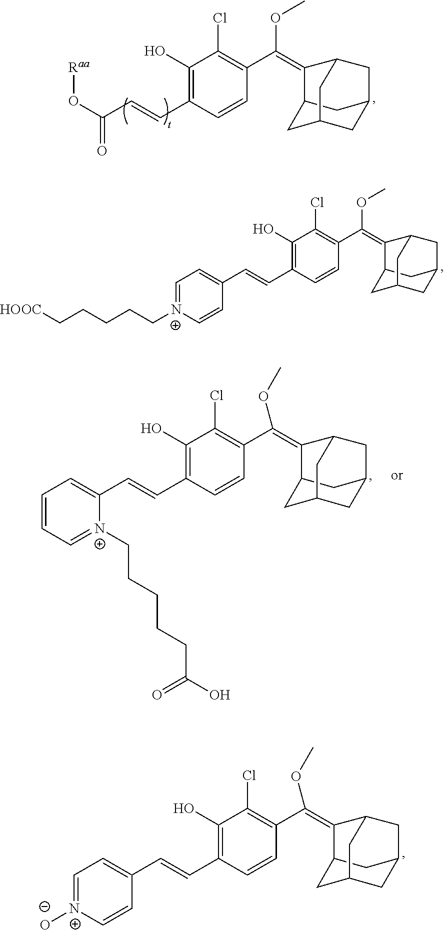

preferably

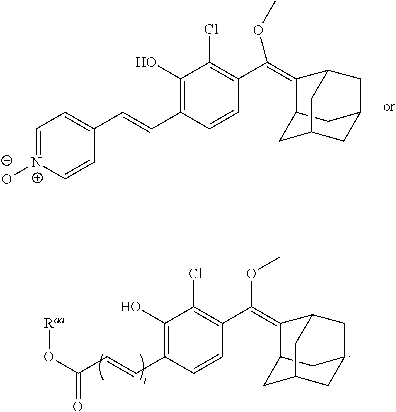

Item 45: The compound according to any one of the preceding items, wherein the compound of Formula Ia has the structure

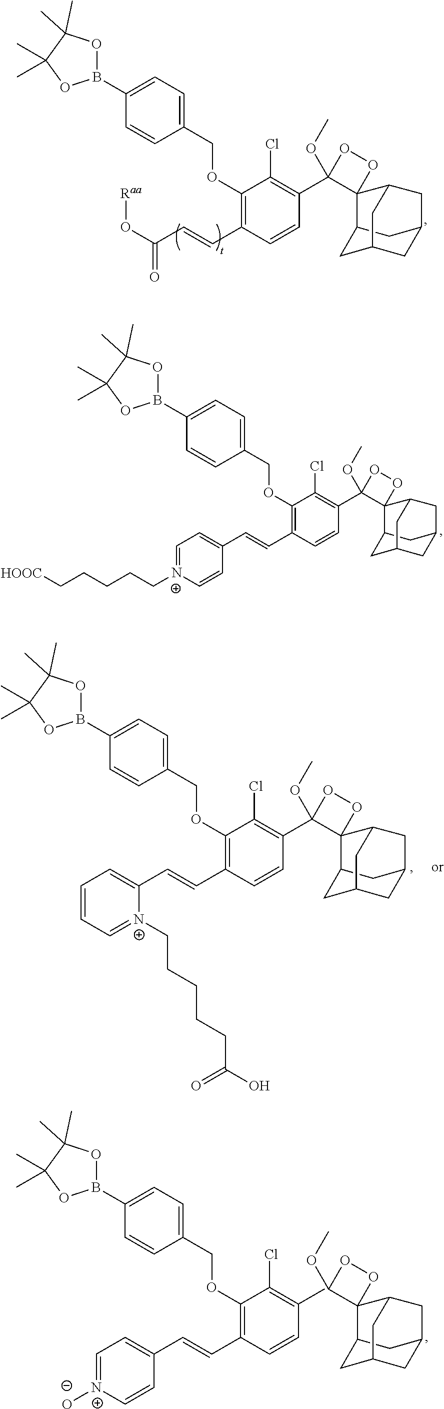

preferably

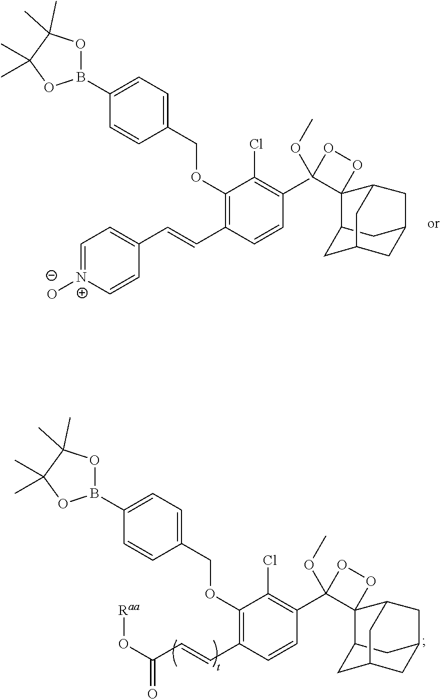

and the compound of Formula Ib has the structure

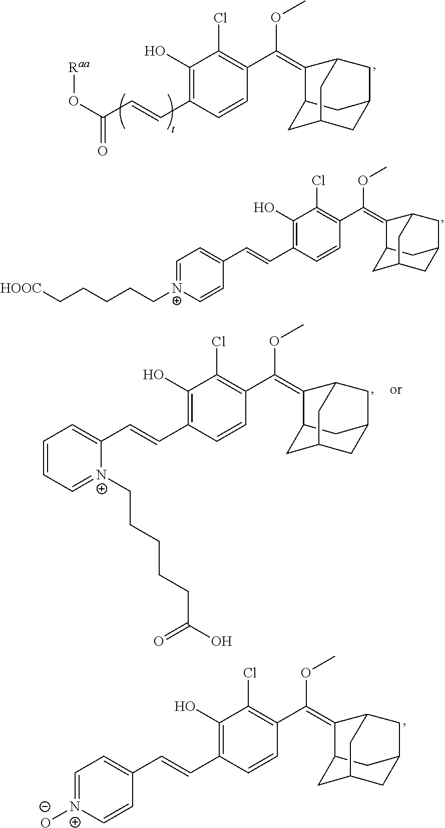

preferably

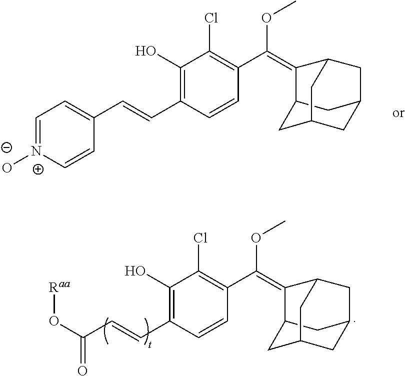

Item 46: A compound of Formula II

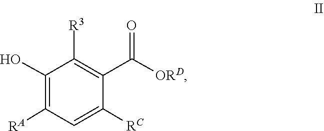

wherein R³, R^{A}, R^{C}, and R^{D} are as defined in the preceding items.

Item 47: The compound of Formula II according to item 46, wherein R³ is —Cl.

Item 48: The compound of Formula II according to item 46 or 47, wherein R^{C} is H and R^{A} is R².

Item 49: The compound of Formula II according to any one of items 46 to 48, wherein R^{D} is methyl.

Item 50: The compound of Formula II according to any one of items 46 to 49 having the structure:

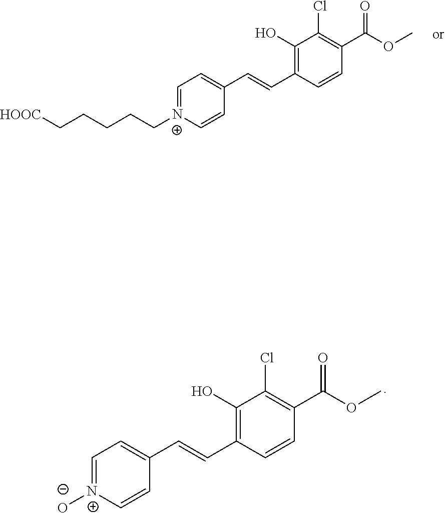 [SMILES: COC(=O)c1ccc(/C=C/c2cc[n+](CCCCCC(=O)O)cc2)c(O)c1Cl ; COC(=O)c1ccc(/C=C/c2cc[n+](O)cc2)c(O)c1Cl]

Item 51: A composition comprising a compound according to any one of items 1-45 and a carrier.

Item 52: A ready-to-use injectable solution comprising a compound according to any one of items 1-45.

Item 53: A compound according to any one of items 1-45, a composition according to item 51 or a ready-to-use injectable solution according to item 52 for use in in vivo diagnostics or in vivo imaging.

Item 54: Use of a compound according to any one of items 1-45 for in vitro imaging.

Item 55: Use of a compound of Formula Ib in an in vitro assay for the detection of singlet oxygen.

Item 56: Use of a compound of Formula Ia in an in vitro assay for the detection of a peroxide, preferably hydrogen peroxide, reactive oxygen species, reactive nitrogen species, or of an enzyme.

Item 57: A method for determining the presence, or measuring the level, of an analyte in a sample, the method comprising the following steps:

(a) contacting the sample with a compound according to any one of items 1-45 thereby converting said compound into an emissive species; and
(b) detecting the emission of said emissive species.

Item 58: The method of item 57, wherein the analyte is an enzyme and R¹ is a group cleavable by said enzyme.

Item 59: The method of item 57, wherein

(i) the analyte is hydrogen peroxide and R¹ is —B(Z″)3⁻Kat⁺ or —B(Z)(Z′), preferably —B(Z)(Z′), more preferably —B(OH)₂ or

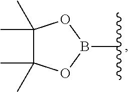 [SMILES: *B1OC(C)(C)C(C)(C)O1]

(ii) the analyte is singlet oxygen and the compound is a compound of Formula Ib, or
(iii) the analyte is reactive oxygen species or reactive nitrogen species and R¹ is selected from the group consisting of

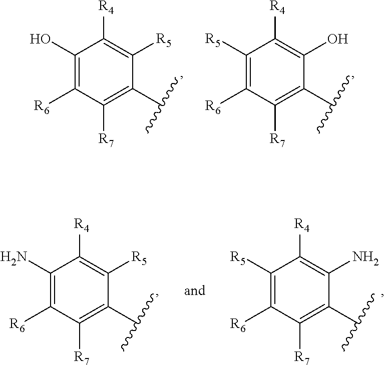

or
(iv) the analyte is a reductase, e.g. a nitroreductase, and R¹ is —NO₂, or azide, or
(v) the analyte is a peptidase and R¹ is selected from the group consisting of

| | | 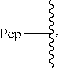 |
|---|---|---|
| amino acidyl having the formula 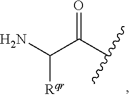 | di-peptidyl having the formula 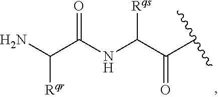 | tri-peptidyl having the formula 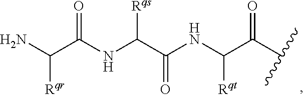 |
| wherein R^{qr} is a side group | wherein R^{qr} and R^{qs} are | wherein R^{qr}, R^{qs}, and R^{qt} |
| depending on the respective | side groups depending on | are side groups depending |
| amino acid, wherein said | the respective amino acids | on the respective amino |
| amino acidyl is preferably | of which the di-peptidyl | acids of which the tri- |
| selected from L-alaninyl, L- | group is composed of, | peptidyl group is |
| leucinyl, and β-alanyl, and | wherein X is preferably | composed of, wherein X is |
| wherein X is preferably | —NH— if L | preferably —NH— if L is |
| —NH— if L is present, | is present, | present, |
| SucOMe-Arg-Pro-Tyrosinyl | L-alanyl (A-), | L-leucinyl (L-), and |
| (SucOMe-RPY-), | | |
| β-alanyl, | | |

or
(vi) the analyte is LacZ and R¹ is beta-D-galactopyranoside.

Item 60: The method of any one of items 57-59, wherein the sample is a biological sample.

Item 61: The method of item 60, wherein the biological sample is a bodily fluid, a bodily fluid-based solution, or a tissue biopsy sample.

Item 62: The method of any one of items 57 to 61, wherein the method is an in vitro method.

Item 63: Use of a compound of any one of items 1 to 45 as a label for a biomolecule, preferably an antibody, a nucleic acid, or a protein.

Item 64: A biomolecule, preferably an antibody, a nucleic acid, or a protein, characterized in that it is bound to a compound of any one of items 1 to 45 as a label.

Item 65: A biomolecule of item 64, preferably an antibody, for use in cancer diagnosis.

The present invention will now be further illustrated by the following, non-limiting example.

### EXAMPLES

General Methods:

All reactions were carried out at room temperature unless stated otherwise. Chemicals and solvents were either A.R. grade or purified by standard techniques. Thin layer chromatography (TLC): silica gel plates Merck 60 F254: compounds were visualized by irradiation with UV light. Column chromatography (FC): silica gel Merck 60 (particle size 0.040-0.063 mm), eluent given in parentheses. Reverse-phase high pressure liquid chromatography (RP-HPLC): C18 5u, 250×4.6 mm, eluent given in parentheses. Preparative RP-HPLC: C18 5u, 250×21 mm, eluent given in parentheses. Fluorescence and chemiluminescence were recorded on Molecular Devices Spectramax i3x.

If not stated otherwise, all chemicals were purchased from Merck and Biosynth AG and used as received.

Abbreviations. AcOH—Acetic acid, MeCN—Acetonitrile, DCM—Dichloromethane, DMF—N,N′-Dimethylformamide, EtOAc—Ethylacetate, Hex—Hexanes, MeOH—Methanol, TFA—Trifluoroacetic acid, THF—Tetrahydrofuran. TIPSCI—Triisopropylsilyl chloride.

### Synthesis Example 1: Synthesis of an Exemplary Compound

Synthesis was carried out according to the following general scheme:

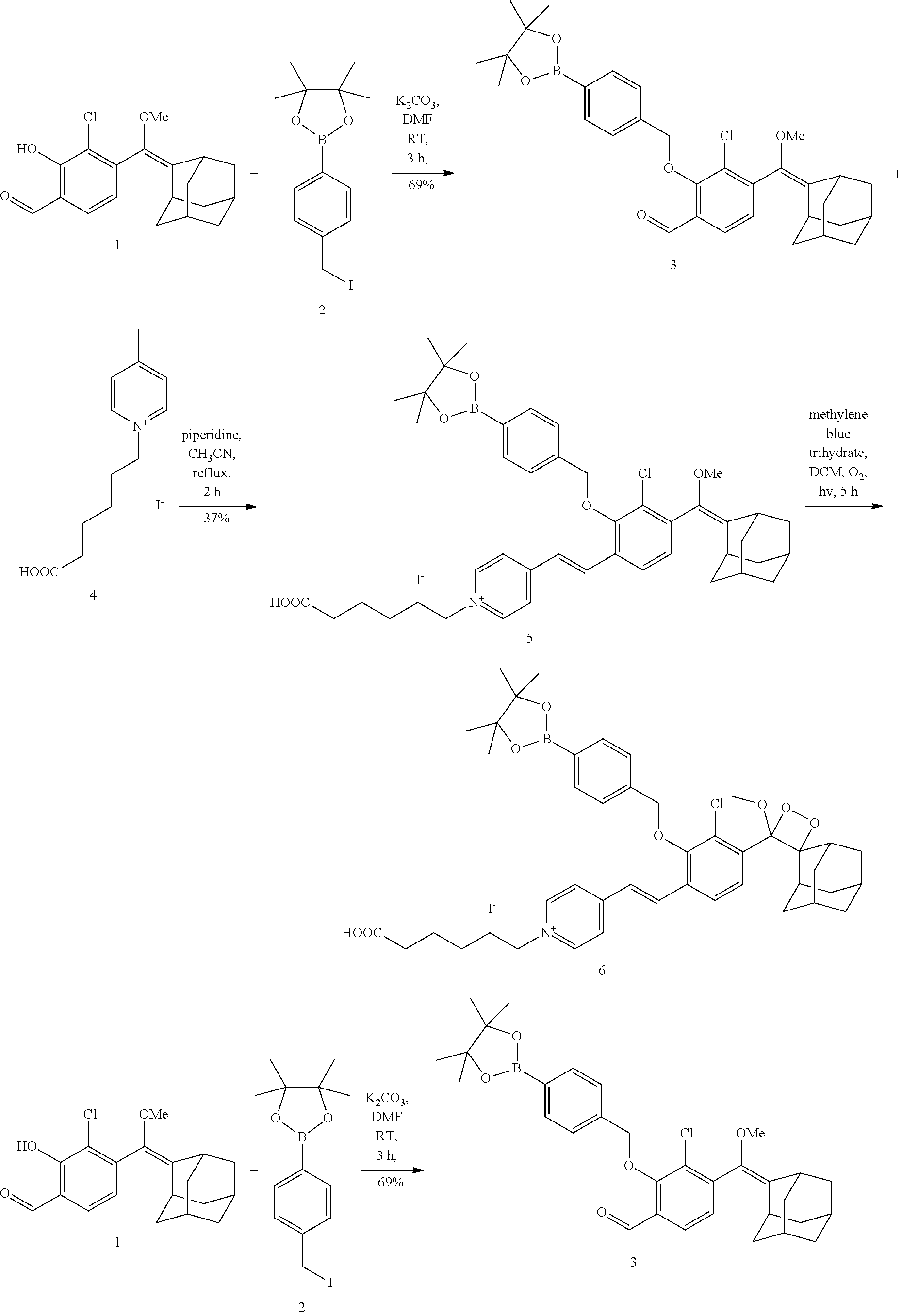

Aldehyde 1 (which was prepared as described in “Chemiluminescent Probes for Activity-Based Sensing of Formaldehyde Released from Folate Degradation in Living Mice”, Angew. Chem. Int. Ed., 2018, vol. 130, issue 25, pages 7630-7634; see Supporting Information) (0.66 mmol, 220 mg) was dissolved in DMF (6.6 mL) and the solution was cooled to 0° C. K₂CO₃ (1.3 eq., 0.86 mmol, 120 mg) was added afterward and the reaction mixture was stirred at RT. Iodide 2 (cf., Karton-Lifshin N., Albertazzi L., Bendikov M., Baran P S., Shabat D., J Am Chem Soc., 2012, 134(50), 20412-20) (1.3 eq., 0.86 mmol, 296 mg) was added and the reaction mixture was stirred at RT for 3 h. The reaction was monitored by TLC (Hex/Et₂O=9:1). After completion, the mixture was cooled to 0° C., diluted with Et₂O (15 mL) and precooled saturated NH₄Cl solution (15 mL) was added. The layers were separated in extraction funnel and the aqueous phase was extracted with Et₂O (2×10 mL). The combined organic layers were washed with brine (20 mL), dried with MgSO₄ and concentrated in vacuo. The crude product was purified by column chromatography (SiO₂, eluent Hex/Et₂O=95:5 to 9:1) to give aldehyde 3 as a yellowish solid (250 mg, 69% yield).

¹H NMR (300 MHz, CDCl₃) δ ppm 1.35 (s, 12H), 1.67-2.08 (m, 13H), 3.28 (bs, 1H), 3.34 (s, 3H) 5.21 (m, 2H), 7.16 (dd, J=7.9, 0.8 Hz, 1H), 7.42 (br d, J=8.0 Hz, 2H), 7.71 (d, J=7.9 Hz, 1H), 7.82 (br d, J=8.1 Hz, 2H), 10.19 (d, J=0.8 Hz, 1H).

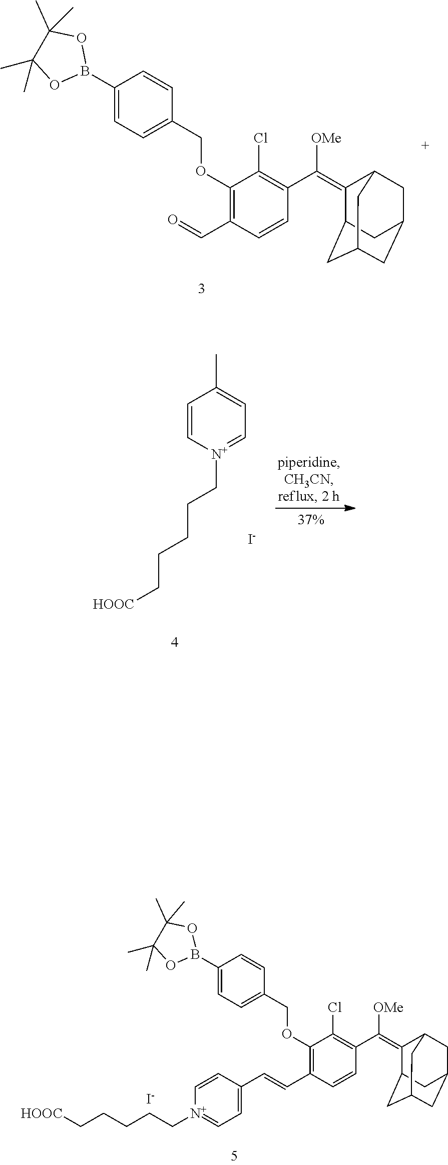 [SMILES: COC(=C1C2CC3CC(C2)CC1C3)c1ccc(C=O)c(OCc2ccc(B3OC(C)(C)C(C)(C)O3)cc2)c1Cl ; Cc1cc[n+](CCCCCC(=O)O)cc1 ; [I-] ; COC(=C1C2CC3CC(C2)CC1C3)c1ccc(/C=C/c2cc[n+](CCCCCC(=O)O)cc2)c(OCc2ccc(B3OC(C)(C)C(C)(C)O3)cc2)c1Cl ; [I-]]

Aldehyde 3 (0.090 mmol, 50 mg) was dissolved in CH₃CN (1 mL) and pyridinium salt 4 (which was prepared according to a protocol described in US 2010/0228008) (1.1 eq., 0.1 mmol, 30 mg) was added, followed by addition of one drop of piperidine (0.3 eq., 0.03 mmol, 2.3 mg, 0.003 mL). This solution was stirred at reflux. After 2h the mixture was concentrated in vacuo. Purification by column chromatography (SiO₂, eluent DCM/MeOH=9:1 to 85:15) gave compound 5 as a yellow solid (0.034 mmol, 25 mg, 37% yield).

¹H NMR (300 MHz, CDCl₃) δ ppm 1.35 (s, 12H), 1.44-2.53 (m, 19H), 3.20-3.37 (m, 6H), 4.77-4.88 (m, 2H), 5.04-5.17 (m, 2H), 7.17 (d, J=8.1 Hz, 1H), 7.29 (d, J=16.5 Hz, 1H), 7.46 (d, J=8.0 Hz, 2H), 7.64 (d, J=8.0 Hz, 1H), 7.78 (d, J=16.5 Hz, 1H), 7.81-7.89 (m, 4H), 9.05 (d, J=6.3 Hz, 2H).

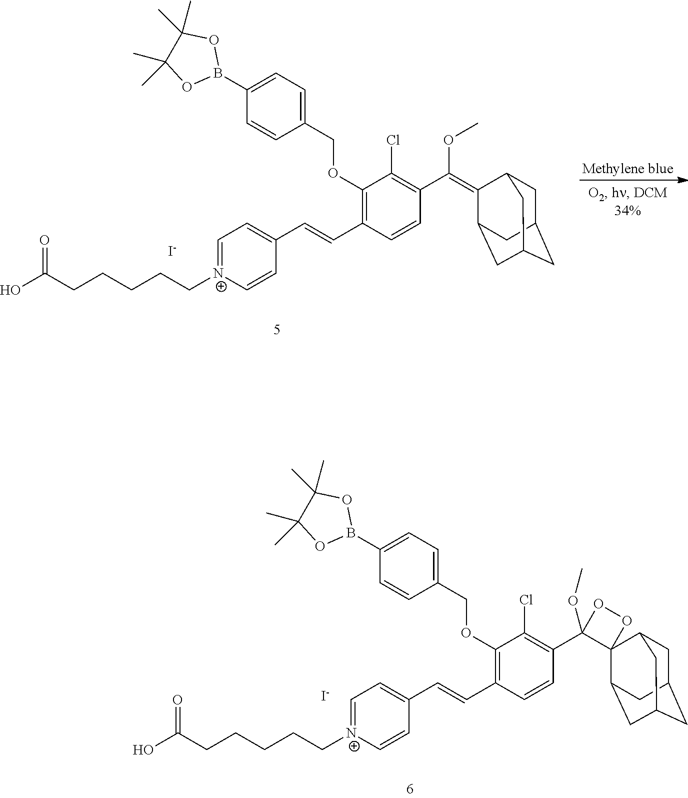 [SMILES: COC(=C1C2CC3CC(C2)CC1C3)c1ccc(/C=C/c2cc[n+](CCCCCC(=O)O)cc2)c(OCc2ccc(B3OC(C)(C)C(C)(C)O3)cc2)c1Cl ; [I-] ; COC1(c2ccc(/C=C/c3cc[n+](CCCCCC(=O)O)cc3)c(OCc3ccc(B4OC(C)(C)C(C)(C)O4)cc3)c2Cl)OOC12C1CC3CC(C1)CC2C3 ; [I-]]

Compound 5 and few milligrams of methylene blue were dissolved in 5 ml of DCM. Oxygen was bubbled through the solution while irradiating with yellow light for 7 minutes. The reaction was monitored by RP-HPLC. After completion, the reaction mixture was concentrated by evaporation under reduced pressure. The crude product was purified by preparative RP-HPLC (gradient of ACN in water (70-100%)).

### Synthesis Example 2: Synthesis of an Exemplary Compound

Synthesis was carried out according to the following general scheme:

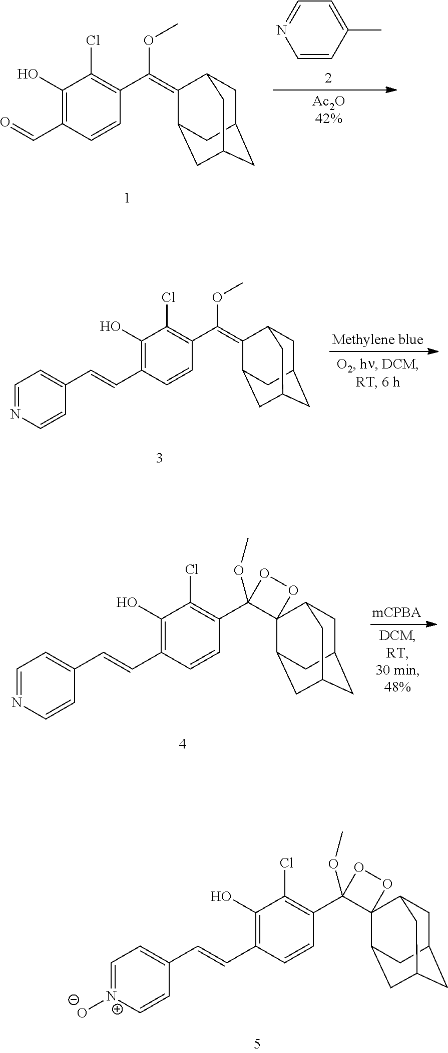

Compound 1 (1.5 mmol, 0.50 g) and 4-methylpyridine (2) (1.53 mmol, 0.143 g) were placed in acetic anhydride and the mixture was stirred at reflux. After 6.5 h the mixture was cooled to RT, MeOH (10 mL) and K₂CO₃ were added and the resulting mixture was stirred at RT. After 2 h the mixture was concentrated in vacuo and saturated aqueous solution of NH₄Cl (20 ml) was added. The resulting mixture was extracted with EtOAc (3 times 20 mL), the combined organics dried and concentrated in vacuo. The crude mixture was purified by column chromatography yielding compound 3 (0.256 g, 42%) as a yellow solid.

¹H NMR (300 MHz, CDCl₃) δ ppm 1.20-2.40 (m, 13H), 3.28 (bs, 1H), 3.33 (s, 3H), 6.88 (d, J=8.0 Hz, 1H), 7.14 (d, J=16.5 Hz, 1H), 7.40 (bd, J=5.4 Hz, 2H), 7.47 (dd, J=8.0 Hz, 0.4 Hz, 1H), 7.62 (d, J=16.5 Hz, 1H), 8.59 (br s, 2H).

Compound 3 (0.049 mmol, 20 mg) was dissolved in CH₂Cl₂ (10 mL) and methylene blue (4 mg) was added. The resulting mixture was stirred at RT while irradiating with Na-light (λ=589 nm) in oxygen atmosphere. After 3 h the mixture was concentrated in vacuo and the residue was purified by preparative TLC (eluent hexane:EtOAc 4:1) yielding dioxetane 4 as a solid (11 mg, 51%).

¹H NMR (300 MHz, CDCl₃) δ ppm 1.20-2.40 (m, 13H), 3.02 (bs, 1H), 3.24 (s, 3H), 7.19 (d, J=16.6 Hz, 1H), 7.41 (d, J=6.0 Hz, 2H), 7.52-7.73 (m, 3H), 8.6 (bd, J=5.3 Hz, 2H).

Compound 4 (0.023 mmol, 10 mg) was dissolved in CH₂Cl₂ (0.5 mL), MCPBA (0.045 mmol, 10 mg) was added and the mixture was stirred at RT. After 30 min the mixture was concentrated in vacuo and the residue was purified by preparative TLC (eluent CH₂Cl₂:MeOH 98:2) yielding N-oxide 5 (5 mg, 48%) as a yellow oil.

¹H NMR (300 MHz, CDCl₃) δ ppm 1.20-2.40 (m, 13H), 3.07 (bs, 1H), 3.28 (s, 3H), 7.20 (d, J=16.6 Hz, 1H), 7.47-7.74 (m, 5H), 8.28 (d, J=7.1 Hz, 2H).

### Synthesis Example 3: Synthesis of an Exemplary Compound

Another exemplary compound was prepared according to the following reaction scheme, wherein the steps are generally performed as set out above.

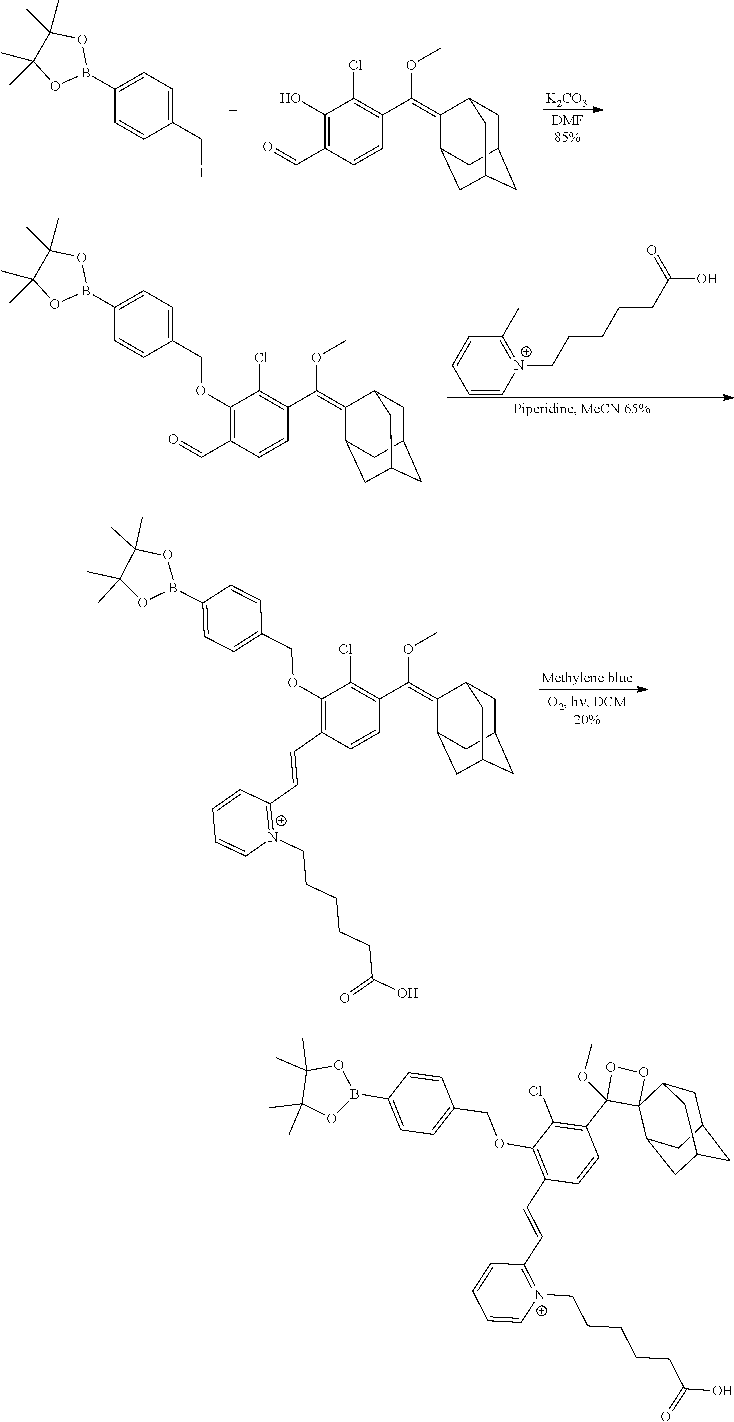

### Synthesis Example 4: Synthesis of CLHP-555

CLHP-555 was synthesized according to the following general scheme:

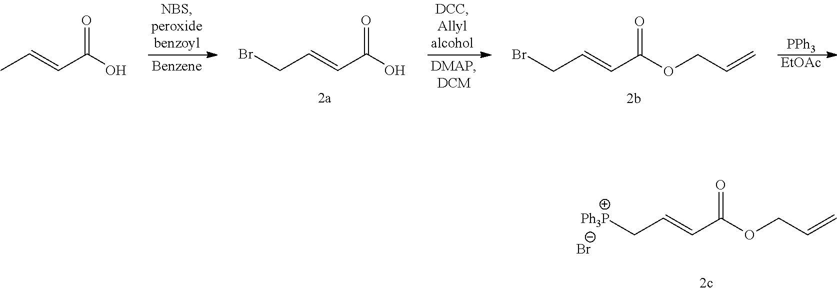

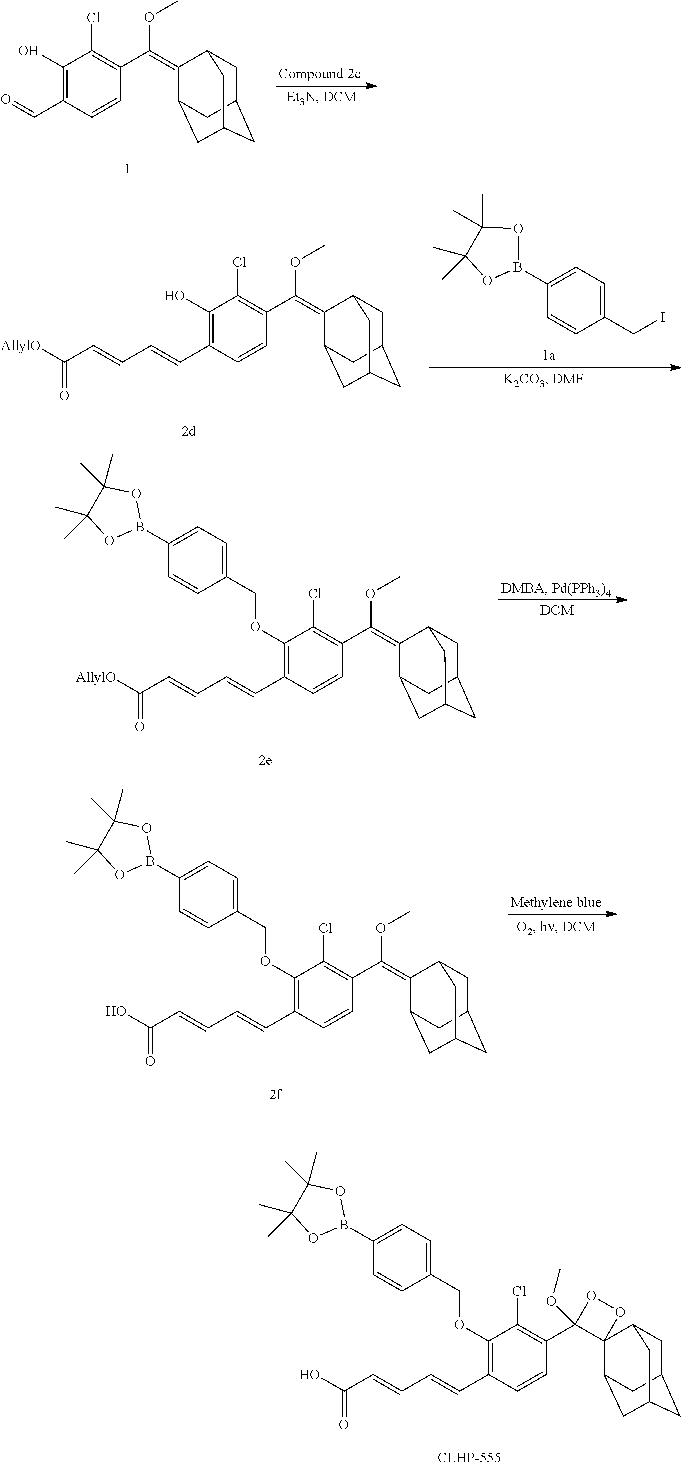

### Compound 2a

A mixture of crotonic acid (1 g, 11.6 mmol), Benzoylperoxide (2.32 mmol) and N-bromosuccinimide (12.76 mmol) in benzene (20 mL) was heated to reflux overnight. The reaction was monitored by TLC (30:70 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (20:80 EtOAc:Hex). The product was obtained as a pale yellow oil (1.16 g, 61% yield). ¹H-NMR (400 MHz, CDCl₃) δ 7.16-7.07 (m, 1H), 6.05 (d, J=15.3 Hz, 1H), 4.03 (dd, J=7.3, 1.2 Hz, 2H). Synthesized according to known procedure: JOC, 76(11), 4467-4481; 2011

### Compound 2b

A mixture of the compound 2a (1 g, 6.1 mmol), DCC (6.7 mmol), 4-DMAP (3.1 mmol) and allyl alcohol (18.3 mmol) in DCM (10 mL) was stirred at room temperature for 1 hour. The reaction was monitored by TLC (5:95 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (5:95 EtOAc:Hex). The product was obtained as a pale yellow oil (1.1 g, 86% yield).

### Compound 2c

A solution of compound 2b (500 mg, 2.5 mmol) in EtOAc (2 mL) was added dropwise to a solution of PPh₃ (2.5 mmol) in ethyl acetate (3 mL). The reaction mixture was stirred at room temperature overnight. The precipitate that formed was removed by filtration, washed with cold ethyl acetate and dried under reduced pressure. The product was obtained as a white solid (640 mg, 55% yield).

### Compound 2d

To a solution of compound 2c (140 mg, 0.3 mmol) and Compound 1 (100 mg, 0.3 mmol) in DCM (5 mL) was added Et₃N (0.6 mmol). The reaction mixture was stirred at room temperature for 15 minutes and monitored by TLC (10:90 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (10:90 EtOAc:Hex). The product was obtained as a pale yellow solid (110 mg, 84% yield).

### Compound 2e

Compound 2d (100 mg, 0.23 mmol) and K₂CO₃ (0.69 mmol) were dissolved in DMF (2 mL) and stirred for 5 minutes at room temperature. Then, Compound Ia (80 mg, 0.23 mmol) was added and the reaction mixture was stirred at room temperature for 1 hour and monitored by TLC (10:90 EtOAc:Hex). Upon completion, the reaction mixture was diluted with EtOAc, and washed with 1M HCl and brine. The organic layer was then dried over Na₂SO₄, filtered, and concentrated under reduced pressure. The crude residue was purified by silica column chromatography (10:90 EtOAc:Hex). The product was obtained as a pale yellow solid (140 mg, 91% yield).

### Compound 2f

A mixture of compound 2e (100 mg, 0.15 mmol), Dimethylbarbituric acid (0.3 mmol) and Pd(PPh₃)₄ (0.02 mmol) in DCM (3 mL) was stirred at room temperature for 1 hour and monitored by TLC (30:70 EtOAc:Hex). Upon full consumption of starting material, the solvent was removed under reduced pressure and the crude residue was filtered using silica column (50:50 EtOAc:Hex). The crude product was reacted without further purification.

### CLHP-555

To a solution of compound 2f (50 mg, 0.08 mmol) in DCM (10 mL) was added a catalytic amount of methylene blue (˜1 mg). Oxygen was bubbled through the solution while irradiating with yellow light for 10 minutes. The reaction was monitored by RP-HPLC (gradient of ACN in water). After completion, the reaction mixture was concentrated under reduced pressure and the crude product was purified by preparative RP-HPLC (gradient of ACN in water). The product was obtained as a pale yellow solid (13 mg, 27% yield).

### Synthesis Example 5: Synthesis of CLHP-595

CLHP-595 was synthesized according to the following general scheme:

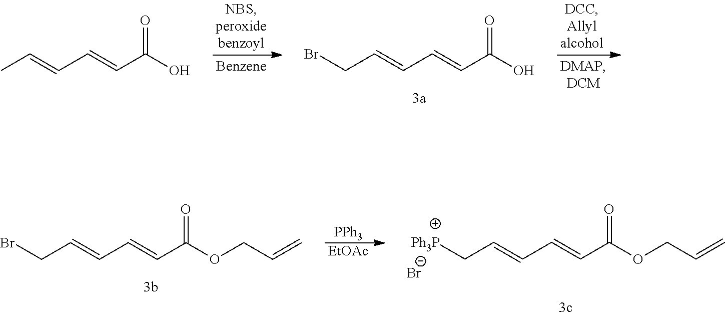

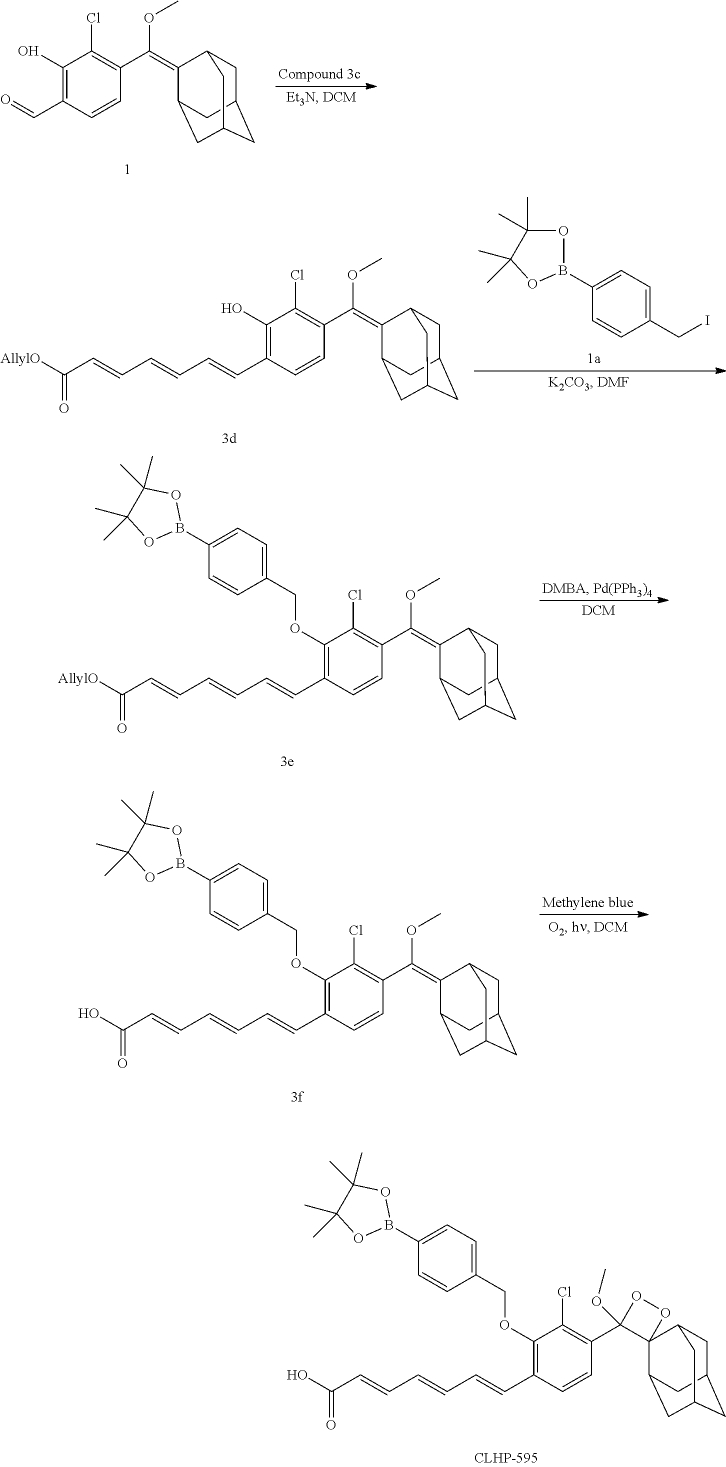

### Compound 3a

A mixture of sorbic acid (1 g, 8.9 mmol), Benzoylperoxide (1.78 mmol) and N-bromosuccinimide (9.79 mmol) in benzene (20 mL) was heated to reflux overnight. The reaction was monitored by TLC (30:70 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (20:80 EtOAc:Hex). The product was obtained as a pale yellow oil (1.18 g, 77% yield). ¹H-NMR (400 MHz, CDCl₃) δ 7.33 (dd, J=15.4, 10.8 Hz, 1H), 6.41 (dd, J=15.1, 10.9 Hz, 1H), 6.29 (dd, J=15.0, 7.4 Hz, 1H), 5.93 (d, J=15.4 Hz, 1H), 4.03 (d, J=7.5 Hz, 2H).

### Compound 3b

A mixture of the compound 3a (1 g, 5.3 mmol), DCC (5.8 mmol), 4-DMAP (2.7 mmol) and allyl alcohol (15.9 mmol) in DCM (10 mL) was stirred at room temperature for 1 hour. The reaction was monitored by TLC (5:95 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (5:95 EtOAc:Hex). The product was obtained as a pale yellow oil (1.0 g, 85% yield). ¹H-NMR (400 MHz, CDCl₃) δ 7.28 (dd, J=15.4, 10.9 Hz, 1H), 6.39 (dd, J=15.0, 10.9 Hz, 1H), 6.25 (dt, J=15.1, 7.6 Hz, 1H), 6.01-5.89 (m, 1H), 5.34 (dd, J=17.2, 1.5 Hz, 1H), 5.25 (dd, J=10.4, 1.2 Hz, 1H), 4.66 (dt, J=5.5, 1.2 Hz, 2H), 4.03 (d, J=7.6 Hz, 2H).

### Compound 3c

A solution of compound 3b (500 mg, 2.2 mmol) in EtOAc (2 mL) was added dropwise to a solution of PPh₃ (2.2 mmol) in ethyl acetate (3 mL). The reaction mixture was stirred at room temperature overnight. The precipitate that formed was removed by filtration, washed with cold ethyl acetate and dried under reduced pressure. The product was obtained as a white solid (650 mg, 60% yield).

### Compound 3d

To a solution of compound 3c (150 mg, 0.3 mmol) and Compound 1 (100 mg, 0.3 mmol) in DCM (5 mL) was added Et₃N (0.6 mmol). The reaction mixture was stirred at room temperature for 15 minutes and monitored by TLC (10:90 EtOAc:Hex). Upon completion, the reaction mixture was diluted with EtOAc, and washed with brine. The organic layer was then dried over Na₂SO₄, filtered, and concentrated under reduced pressure. The crude residue was purified by silica column chromatography (10:90 EtOAc:Hex). The product was obtained as a pale yellow solid (113 mg, 81% yield).

### Compound 3e

Compound 3d (100 mg, 0.21 mmol) and K₂CO₃ (0.69 mmol) were dissolved in DMF (2 mL) and stirred for 5 minutes at room temperature. Then, Compound Ia (75 mg, 0.21 mmol) was added and the reaction mixture was stirred at room temperature for 1 hour and monitored by TLC (10:90 EtOAc:Hex). Upon completion, the reaction mixture was diluted with EtOAc, and washed with 1M HCl and brine. The organic layer was then dried over Na₂SO₄, filtered, and concentrated under reduced pressure. The crude residue was purified by silica column chromatography (10:90 EtOAc:Hex). The product was obtained as a pale yellow solid (110 mg, 77% yield).

### Compound 3f

A mixture of compound 3e (100 mg, 0.15 mmol), Dimethylbarbituric acid (0.3 mmol) and Pd(PPh₃)₄ (0.02 mmol) in DCM (3 mL) was stirred at room temperature for 1 hour and monitored by TLC (30:70 EtOAc:Hex). Upon full consumption of starting material, the solvent was removed under reduced pressure and the crude residue was filtered using silica column (50:50 EtOAc:Hex). The crude product was reacted without further purification.

### CLHP-595

To a solution of compound 3f (50 mg, 0.08 mmol) in DCM (10 mL) was added a catalytic amount of methylene blue (˜1 mg). Oxygen was bubbled through the solution while irradiating with yellow light for 10 minutes. The reaction was monitored by RP-HPLC (gradient of ACN in water). After completion, the reaction mixture was concentrated under reduced pressure and the crude product was purified by preparative RP-HPLC (gradient of ACN in water). The product was obtained as a pale yellow solid (25 mg, 46% yield). ¹H-NMR (400 MHz, CDCl₃) δ 7.85 (m, 3H), 7.58-7.37 (m, 4H), 6.93 (m, 2H), 6.75-6.62 (m, 1H), 6.46 (dd, J=14.5, 11.5 Hz, 1H), 5.94 (d, J=15.2 Hz, 1H), 4.94 (s, 2H), 3.22 (s, 3H), 3.02 (s, 1H), 2.32 (d, J=11.9 Hz, 1H), 2.03 (s, 1H), 1.93-1.43 (m, 10H), 1.36 (s, 12H). ¹³C-NMR (101 MHz, CDCl₃) δ 171.91, 153.34, 146.23, 141.46, 139.08, 135.04, 133.45, 133.11, 131.38, 131.15, 130.38, 128.82, 127.53, 124.25, 120.79, 111.89, 96.42, 83.94, 75.68, 49.69, 36.62, 33.89, 33.59, 32.62, 32.28, 31.60, 26.19, 25.83, 24.86. MS (ES−): m/z calc. for C₃₈H₄₄BClO₈: 674.3; found: 673.6 [M-H]⁻.

### Example 1

### Chemiluminescent Properties of Compound Ia1

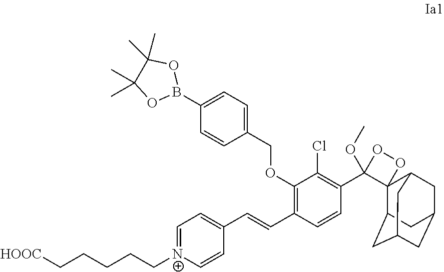 [SMILES: COC1(c2ccc(/C=C/c3cc[n+](CCCCCC(=O)O)cc3)c(OCc3ccc(B4OC(C)(C)C(C)(C)O4)cc3)c2Cl)OOC12C1CC3CC(C1)CC2C3]

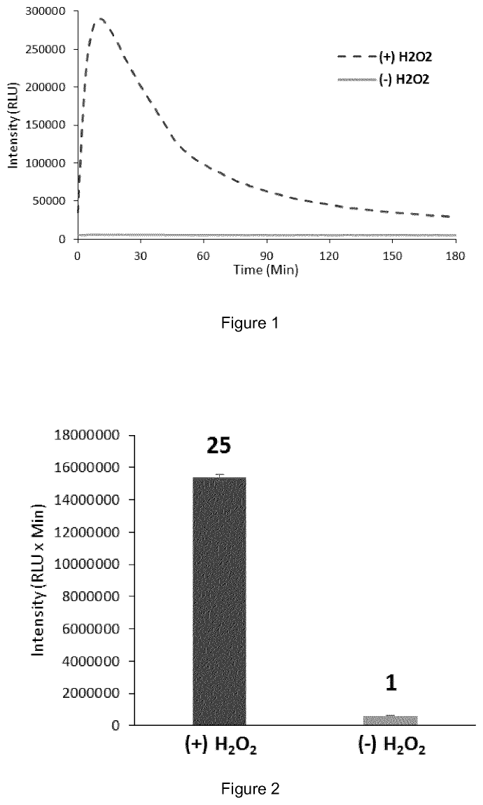

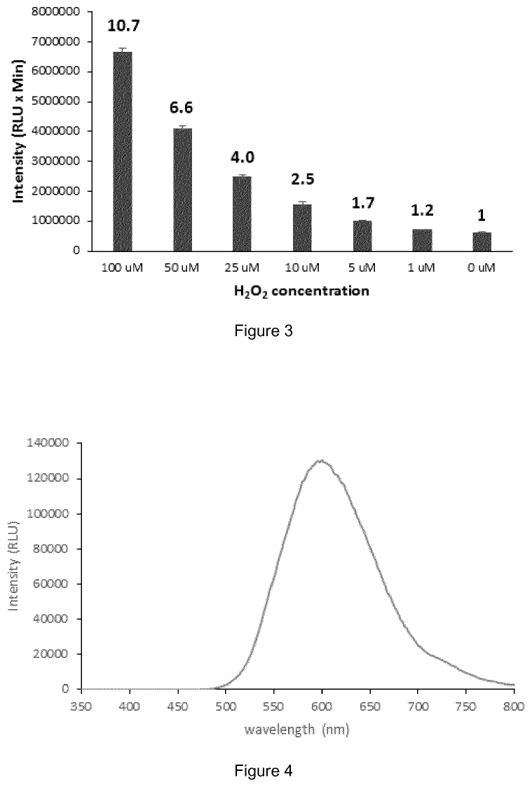

Upon reaction with H₂O₂ [1 mM] in PBS (pH 7.4) (10% DMSO) at 37° C., compound Ia1 (100 μM) shows a red emission with an emission maximum at 660 nm. The respective chemiluminescent kinetic profile is shown in FIG. 1 and the total light emission with [1 mM] or without the presence of H₂O₂ is shown in FIG. 2. The chemiluminescent response to various H₂O₂ concentrations in PBS (pH 7.4) (1% DMSO) is shown in FIG. 3.

### Example 2

### Chemiluminescent Properties of Compound Ia2

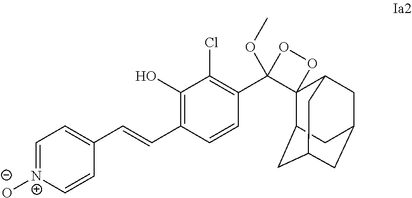 [SMILES: COC1(c2ccc(/C=C/c3cc[n+]([O-])cc3)c(O)c2Cl)OOC12C1CC3CC(C1)CC2C3]

Chemiluminescent emission spectrum of compound Ia2 [100 μM] in PBS (pH 7.4) (10% DMSO) is shown in FIG. 4. Figure shows that compound Ia2 shows an emission maximum at about 590 to 600 nm. The emission was so intense that it was visible by the naked eye.

### Example 3

### Chemiluminescent Properties of Compound Ia3

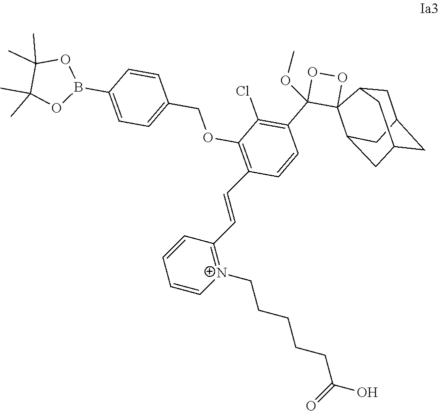 [SMILES: COC1(c2ccc(/C=C/c3cccc[n+]3CCCCCC(=O)O)c(OCc3ccc(B4OC(C)(C)C(C)(C)O4)cc3)c2Cl)OOC12C1CC3CC(C1)CC2C3]

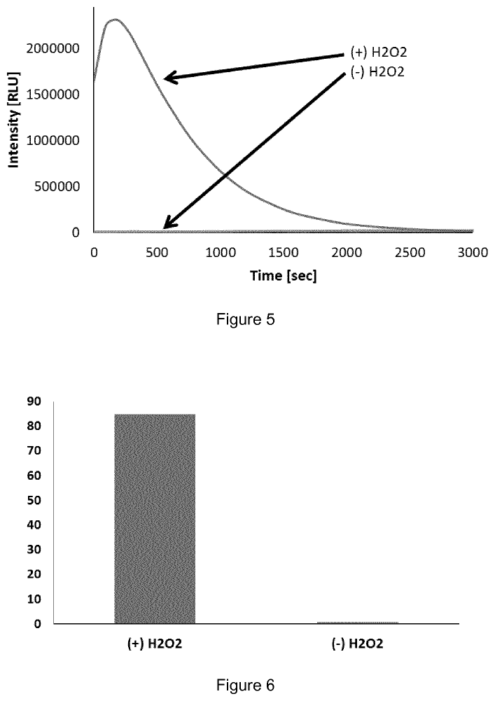

Upon reaction with H₂O₂ [1 mM] in PBS (pH 7.4) (10% DMSO) at 26° C., compound Ia3 (100 μM) shows an emission with a maximum at 650 nm. The chemiluminescent kinetic profile is shown in FIG. 5 and the total light emission with and without the presence of H₂O₂ is shown in FIG. 6.

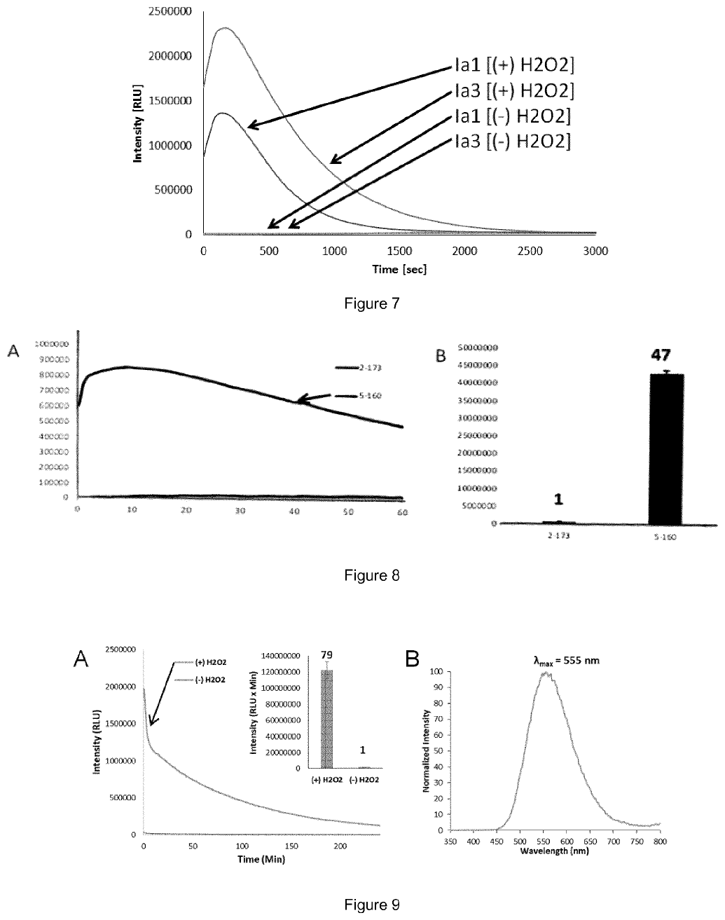

FIG. 7 shows a comparison of the chemiluminescent kinetic profiles of compounds Ia1 and Ia3 ([100 μM] with and without H₂O₂ [1 mM] in PBS, pH=7.4, (10% DMSO) at 26° C.).

### Example 4

### Comparison of Luminescence Properties of Compounds SAG 2-173 and OG 5-160

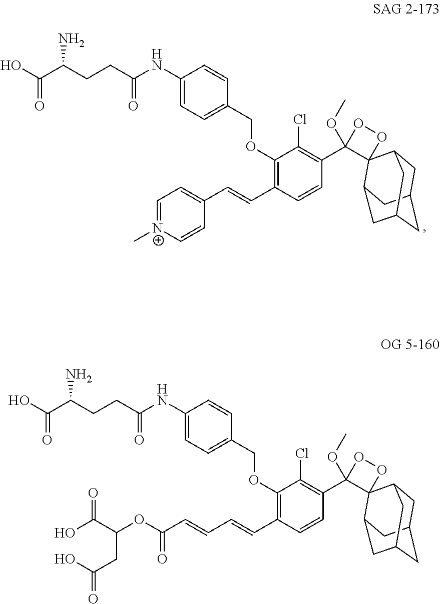 [SMILES: COC1(c2ccc(/C=C/c3cc[n+](C)cc3)c(OCc3ccc(NC(=O)CC[C@@H](N)C(=O)O)cc3)c2Cl)OOC12C1CC3CC(C1)CC2C3 ; COC1(c2ccc(/C=C/C=C/C(=O)OC(CC(=O)O)C(=O)O)c(OCc3ccc(NC(=O)CC[C@@H](N)C(=O)O)cc3)c2Cl)OOC12C1CC3CC(C1)CC2C3]

Luminescence properties of compounds SAG 2-173 and OG 5-160 [100 μM] were recorded in PBS buffer, pH 7.4, 10% DMSO in the presence of gamma-glutamyltransferase (GGT) (1U/mL) at 37° C.

The chemiluminescence kinetic profile is shown in FIG. 8A and the total light emission of both compounds id shown in FIG. 8B.

It was surprisingly found that compound OG 5-160 is 47 times more emissive than compound SAG 2-173.

### Example 5

### Luminescence Properties of Compounds CLHP-555 and CLHP-595

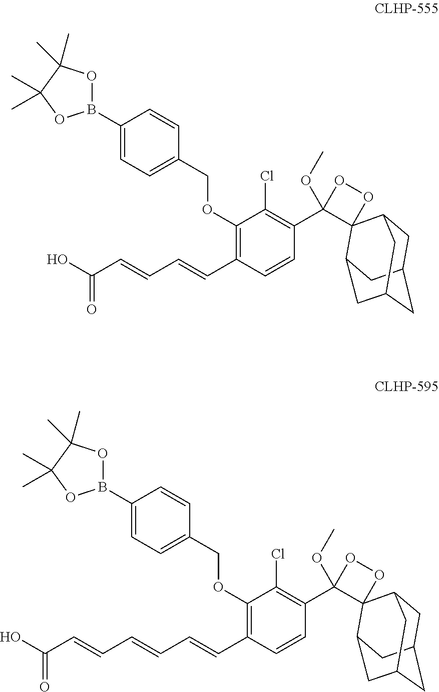 [SMILES: COC1(c2ccc(/C=C/C=C/C(=O)O)c(OCc3ccc(B4OC(C)(C)C(C)(C)O4)cc3)c2Cl)OOC12C1CC3CC(C1)CC2C3 ; COC1(c2ccc(/C=C/C=C/C=C/C(=O)O)c(OCc3ccc(B4OC(C)(C)C(C)(C)O4)cc3)c2Cl)OOC12C1CC3CC(C1)CC2C3]

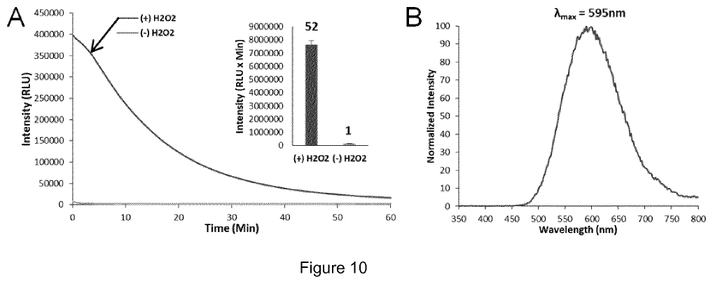

The chemiluminescent properties are shown in FIGS. 9 and 10.

FIG. 9A shows the chemiluminescent kinetic profile of CLHP-555 [10 μM] with and without H₂O₂ [100 μM] in PBS, pH=7.4, (10% DMSO). The inset show S/N ratio of total light emission.

FIG. 9B shows the chemiluminescence emission spectrum of CLHP-555 [100 μM] with H₂O₂ [1000 μM] in PBS, pH=7.4, (10% DMSO).

FIG. 10A shows the chemiluminescent kinetic profile of CLHP-595 [10 μM] with and without H₂O₂ [100 μM] in PBS, pH=7.4, (10% DMSO). The inset show S/N ratio of total light emission.

FIG. 10B shows the chemiluminescence emission spectrum of CLHP-595 [100 μM] with H₂O₂ [1000 μM] in PBS, pH=7.4, (10% DMSO).

1.-21. (canceled)

22. A compound of the Formula Ia or Ib 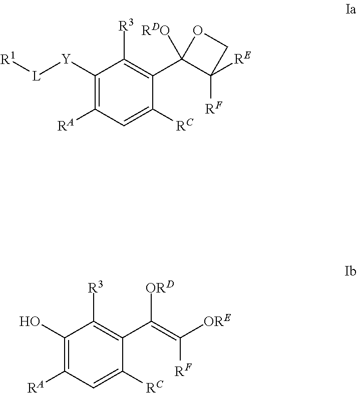
wherein
R^{D} is selected from the group consisting of a linear or branched C1-C18 alkyl and C3-C7 cycloalkyl, preferably R^{D} is methyl or ethyl, more preferably methyl;
R^{E} and R^{F} are independently selected from the group consisting of a branched C3-C18 alkyl and C3-C7 cycloalkyl, or R^{E} and R^{F} together with the carbon atom to which they are attached form a fused, spiro or bridged cyclic or polycyclic ring, preferably adamantyl, wherein the adamantyl may be substituted;
R³ is —H, —F, —Cl, —Br, —I, —CF₃, —NO₂, —CN, —COOR^{XX}, —C(O)R^{XX}, —SO₂R^{XX} or R², preferably R³ is —Cl;
R^{A} and R^{C} are independently selected from the group consisting of —H, —F, —Cl, —Br, —I, —CF₃, —NO₂, —CN, —R^{x}COOR^{XX}, —COOR^{XX}, —C(O)R^{XX}, —SO₂R^{XX} and R²;
R^{X} is linear or branched C1-C6 alkylene or linear or branched C1-C6 alkenylene, preferably —CH═CH—;
R^{XX} is linear or branched C1-18 alkyl, C2 to C8 alkenyl or C2 to C8 alkynyl chain or —H;
provided that at least one, preferably one, of R³, R^{A} and R^{C} is R², and more preferably that R³ is as defined above and R^{A} is R² and R^{C} is H, or that R³ is as defined above and R^{A} is H and R^{C} is R²;
R² is selected from the group consisting of 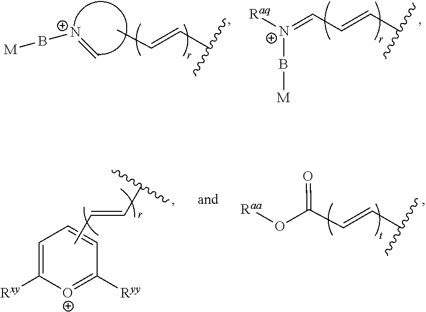
wherein 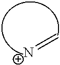
denotes a mono- or polycyclic, aromatic or nonaromatic ring system comprising the moiety 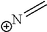
as a ring member,
wherein the moiety 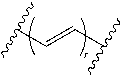
is connected to 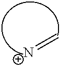
via an atom, which is a member of said mono- or polycyclic, aromatic or nonaromatic ring system,
provided that a delocalized π-system extends from the positively charged nitrogen atom of 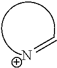
via moiety 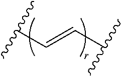
to the central aromatic ring of the compound of Formula Ia or Ib,
wherein each ring of said mono- or polycyclic, aromatic or nonaromatic ring system may be substituted with one or more groups selected from the group consisting of —OH, —CN, —SO₃⁻, linear or branched C1-C6 alkyl, linear or branched C2-C6 alkenyl, linear or branched C2-C6 alkynyl, a polyethylene glycol chain, and a polypropylene glycol chain,
wherein 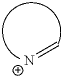
is optionally substituted with one or two negatively charged substituent(s), preferably selected from the group consisting of —COO⁻ and —SO₃⁻, in ortho position to the positively charged nitrogen atom,
r is selected from the group consisting of 1, 2, 3, 4, 5, and 6, preferably r is 1,
R^{xy} is selected from the group consisting of H, linear or branched C1-C6 alkyl, linear or branched C2-C6 alkenyl, linear or branched C2-C6 alkynyl, and C3-C7 cycloalkyl, preferably from methyl, ethyl, propyl, isopropyl, butyl, sec-butyl, and tert-butyl,
R^{yy} is selected from the group consisting of H, methyl, ethyl, n-propyl, isopropyl, n-butyl, sec-butyl, n-pentyl, linear or branched C2-C6 alkenyl, linear or branched C2-C6 alkynyl, and C3-C7 cycloalkyl groups, preferably from methyl, ethyl, propyl, isopropyl, butyl, and sec-butyl,
R^{aq} is a linear or branched C1 to C8 alkyl, preferably C2 to C6 alkyl, a linear or branched C2 to C8 alkenyl, a linear or branched C2 to C8 alkynyl, or a linear or branched C4 to C12 heteroalkyl, wherein the linear or branched C1 to C8 alkyl, C2 to C8 alkenyl, C2 to C8 alkynyl, or C4 to C12 heteroalkyl may be substituted with one or more groups selected from the group consisting of —OH, —COOH, halogen, preferably —Cl or —F, and —NH₂, and wherein the linear or branched C1 to C8 alkyl, C2 to C8 alkenyl or C2 to C8 alkynyl chain may comprise one or more —O— or —CO— groups within the chain,
M is an optionally present group,
wherein, if M is absent, B is —O^{⊖}, H, a linear or branched C2 to C8 alkyl, preferably a linear or branched C2 to C6 alkyl, a linear or branched C2 to C8 alkenyl or a linear or branched C2 to C8 alkynyl chain,
wherein the linear or branched C2 to C8 alkyl, C2 to C8 alkenyl or C2 to C8 alkynyl chain may be substituted with one or more groups selected from the group consisting of —OH, —COOH, halogen, preferably —Cl or —F, —NH₂, and a group capable of binding to an amino, carboxy, or mercapto group of a peptide, endolysine, or protein, thus allowing for binding said peptide, endolysine, or protein to B; and
wherein the linear or branched C2 to C8 alkyl, C2 to C8 alkenyl or C2 to C8 alkynyl chain may comprise one or more —O— or —CO— groups within the chain,
preferably B is —O^{⊕}, H, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —CH═CH₂, —CH═CHCH₃, —CH₂CH═CH₃, or a linear or branched C4-C8 alkenyl group,
preferably, if M is absent and B is H, 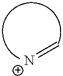
is substituted with one or two, preferably two, —COO⁻ groups in ortho position to the positively charged nitrogen atom,
or wherein, if M is present, B is a linear or branched C1 to C8 alkylene, preferably C2 to C6 alkylene, a linear or branched C2 to C8 alkenylene or linear or branched C2 to C8 alkynylene chain,
wherein the linear or branched C1 to C8 alkylene, C2 to C8 alkenylene or C2 to C8 alkynylene chain may be substituted with one or more groups selected from the group consisting of —OH, —COOH, halogen, preferably —Cl or —F, —NH₂, and a group capable of binding to an amino, carboxy, or mercapto group of a peptide, endolysine, or protein, thus allowing for binding said peptide, endolysine, or protein to B; and
wherein the linear or branched C1 to C8 alkylene, C2 to C8 alkenylene or C2 to C8 alkynylene chain may comprise one or more —O— or —CO— groups within the chain,
preferably B is —CH₂—, —(CH₂)₂—, —(CH₂)₃—, —(CH₂)₄—, —(CH₂)₅—, —(CH₂)₆—, —(CH₂)₇—, —(CH₂)₈—, —CH═CH—, —CH₂CH═CHCH₂—, a linear or branched C6 alkenylene group with one or two double bonds or a linear or branched C8 alkenylene group with one, two or three double bonds, and
M is selected from the group consisting of cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, tetrazole, aryl, alkenyl, 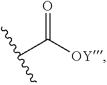
carbonyl having the structure 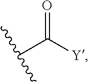
amide, and an amide having the
structure 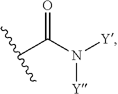
or M is a moiety including one or more groups selected from the group consisting of cyano, nitro, sulfoxide, sulfon, sulfonic acid, phosphonic acid, amine (primary, secondary, tertiary), imine, hydrazine, amidine, guanidine, hydroxyl, carboxyl, β-dicarbonyl, sulfonamide, sulfonylurea, imide, and tetrazole, aryl, alkenyl, 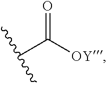
carbonyl having the structure 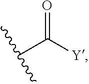
amide, and an amide having the structure 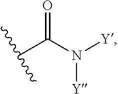
wherein Y′″ is —H, an C1-C8 alkyl, C2-C8 alkenyl, C2-C8 alkynyl, an alkali metal ion or a negative charge,
wherein Y′ and Y″ are independently selected from the group consisting of —H, an C1-C8 alkyl, C2-C8 alkenyl, and an C2-C8 alkynyl,
or Y′ and Y″ together with the nitrogen atom to which they are attached form a heterocyclic structure, preferably a maleimide group;
preferably M is —COOH, —SO₃⁻, a moiety derived from an amino acid, a moiety derived from a monosaccharide or a disaccharide, a moiety derived from a polycarboxylic acid, a moiety derived from polyethylene glycol or polypropylene glycol, or a moiety derived from a polyol, more preferably —COOH or —SO₃⁻,
t is 2, 3, or 4;
R^{aa} is —H, a linear or branched C1-C6 alkyl, preferably methyl or ethyl, more preferably methyl, a moiety derived from an amino acid, a moiety derived from a monosaccharide or a disaccharide, a moiety derived from a polycarboxylic acid, a moiety derived from polyethylene glycol or polypropylene glycol, a moiety derived from a polyol, or a cell membrane-permeable group selected from the group consisting of penetratin, transportan, HIV1-Tat-Peptide₄₈₋₆₀, HIV1-Rev-Peptide₃₄₋₅₀, antennapedia₄₃₋₅₈, octaarginine, choline, a moiety bound to choline, (CH₃)₃N⁺(CH₂)₂—, acetoxymethyl ester derivative of a carboxylic acid, and a moiety comprising one or more acetoxymethyl ester derivatives of a carboxylic acid;
Y is absent or is —O—, provided that Y is absent if R¹ is —B(Z)(Z′) or —B(Z″)₃⁻Kat⁺ and L is absent,
wherein Z and Z′ are independently selected from the group consisting of R^{ab} and OR^{ac},
wherein R^{ab} is selected from the group consisting of —OH, —O⁻Kat+, C1-C4 alkyl, C2-C4 heteroalkyl, C2-C4 alkenyl, C2-C4 heteroalkenyl, C2-C4 alkynyl, C2-C4 heteroalkynyl, C5-C6 aryl, C5-C6 heteroaryl, C6-C10 aralykl, and C6-C10 heteroaralkyl,
and R^{ac} is selected from the group consisting of —H, C1-C4 alkyl, C2-C4 heteroalkyl, C2-C4 alkenyl, C2-C4 heteroalkenyl, C2-C4 alkynyl, C2-C4 heteroalkynyl, C5-C6 aryl, C5-C6 heteroaryl, C6-C10 aralykl, and C6-C10 heteroaralkyl, or
wherein two R^{ab}, two R^{ac} or one R^{ab} and one R^{ac} together with their intervening atoms form a 5- to 7-membered heterocyclic ring, preferably a saturated heterocyclic ring;
Z″ is selected from the group consisting of —F, —Cl, —Br, and —I, preferably Z″ is —F;
Kat⁺ is an organic or inorganic cation, preferably an alkali metal cation;
L is absent or is a linker selected from the group consisting of moieties L1 to L8 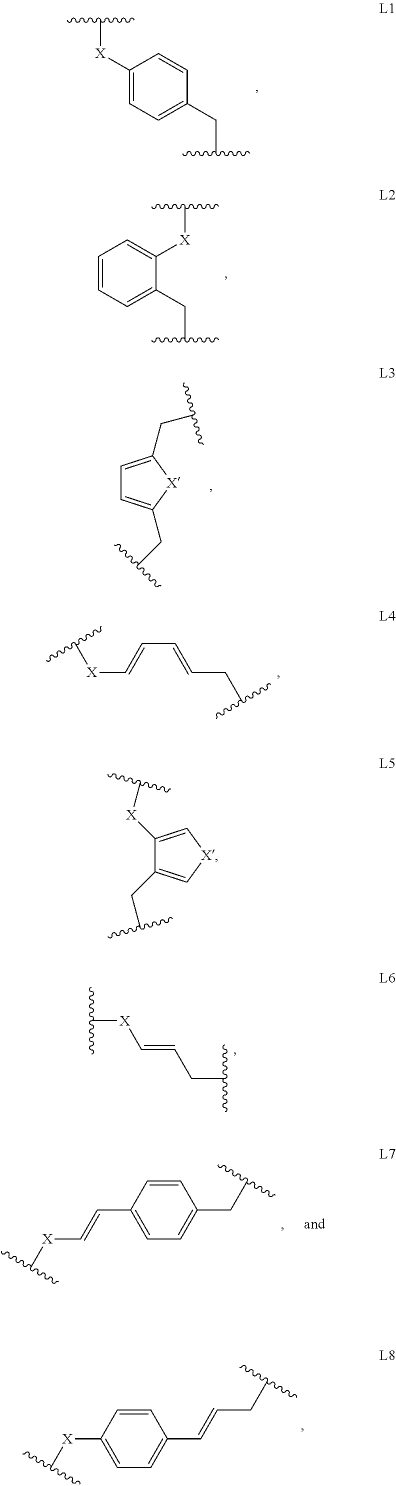
wherein
X is absent or is —O—, —NH—, —NR^{G}—, —S—, or —NH—COO— wherein the COO-moiety is bound to R¹, wherein R^{G} is selected from a substituted or unsubstituted C1-C12 alkyl, preferably X is absent or is —O— or —NH—,
provided that X is absent if R¹ is —B(Z)(Z′), —B(Z″)₃⁻Kat+, —NO₂ or an azide group,
X′ is selected from the group consisting of S, O, NH, and —NR^{G}—, wherein R^{G} is selected from a substituted or unsubstituted C1-C12 alkyl,
X is connected to R¹,
wherein each of L1 to L8 is optionally functionalized with a group capable of binding to an amino, carboxy, or mercapto group of a peptide, endolysine, or protein, or a cell membrane-permeable group selected from the group consisting of penetratin, transportan, HIV1-Tat-Peptide₄₈₋₆₀, HIV1-Rev-Peptide₃₄₋₅₀, antennapedia₄₃₋₅₈, octaarginine, choline, a moiety bound to choline, (CH₃)₃N⁺(CH₂)₂—, acetoxymethyl ester derivative of a carboxylic acid, and a moiety comprising one or more acetoxymethyl ester derivatives of a carboxylic acid, thus allowing for binding said peptide, endolysine, or protein to L,
provided that L is absent and R¹ is —B(Z)(Z′), —B(Z″)₃⁻Kat⁺ if Y is absent, and
provided that Y is —O— if L is present,
R¹ is an analyte-responsive group capable of reacting with an analyte, wherein
if L is present and X is present, then X—R¹ is converted into a XH group upon reaction of R¹ with said analyte, or
if L is present and X is absent, then R¹ is converted into a π-donor group upon reaction of R¹ with said analyte, or
if L and Y are absent and R¹ is —B(Z)(Z′) or —B(Z″)₃⁻Kat⁺, then R¹ is converted into a —OH group, or
if L is absent and Y is —O—, then the —O—R¹ moiety is converted into a —OH group.

23. The compound according to claim 22, wherein R² is selected from the group consisting of 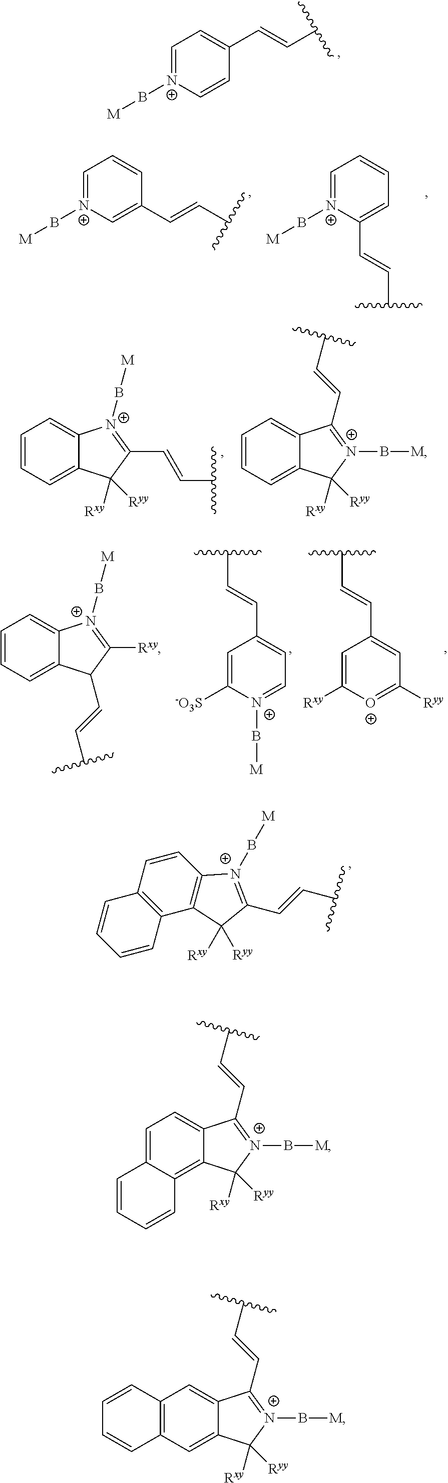 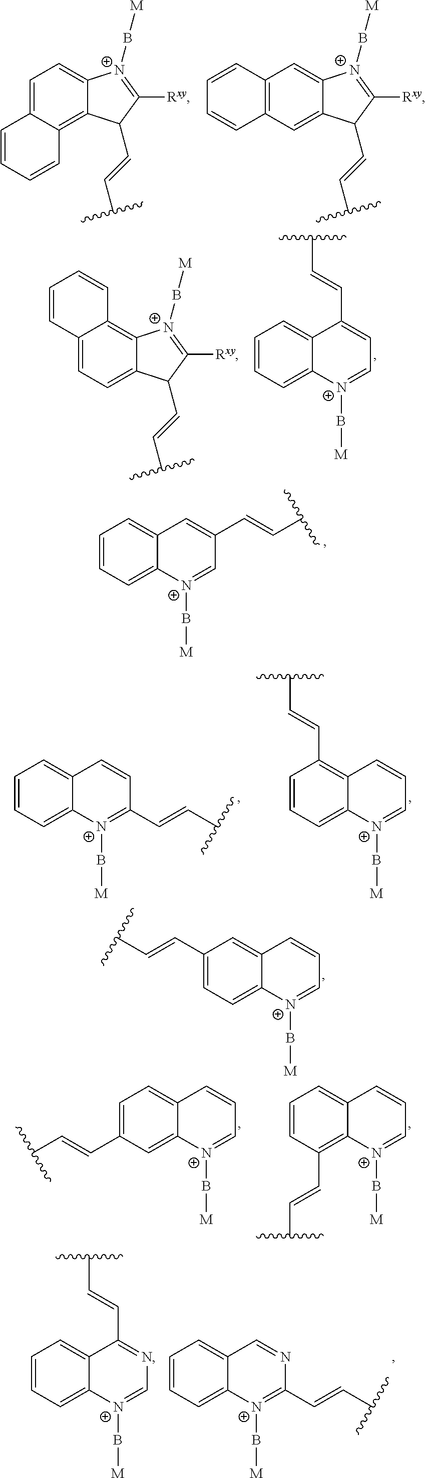 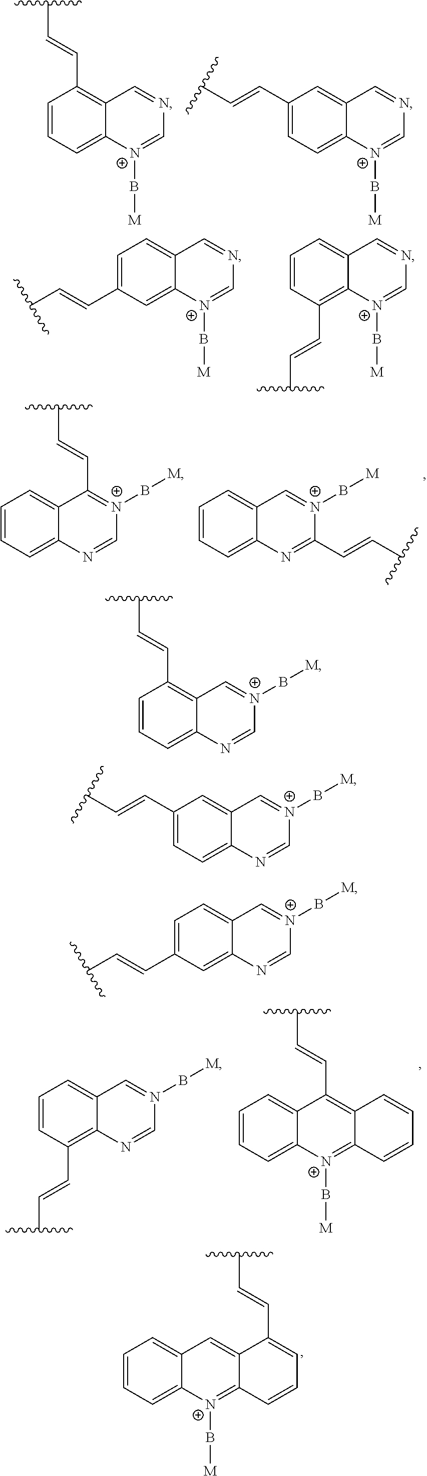 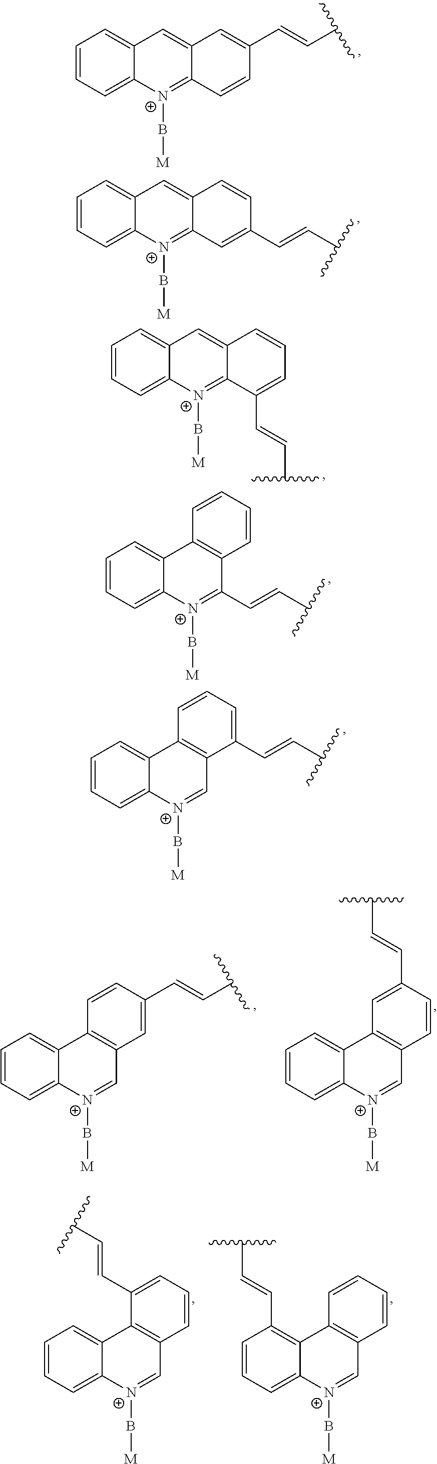 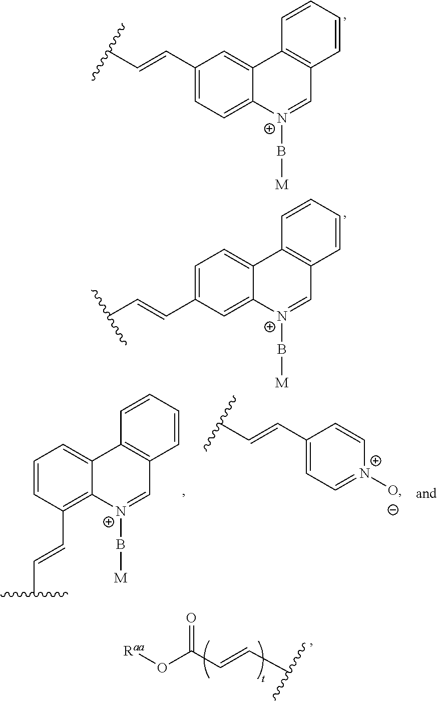
wherein the aromatic ring(s) of R² may be substituted with one or more groups selected from the group consisting of —OH, —CN, —SO₃⁻, linear or branched C1-C6 alkyl, linear or branched C2-C6 alkenyl, and linear or branched C2-C6 alkynyl, a polyethylene glycol chain, and a polypropylene glycol chain,
wherein, if the respective position is available for substitution, the aromatic ring is optionally substituted with one or two negatively charged substituent(s), preferably selected from the group consisting of —COO⁻ and —SO₃⁻, in ortho position to the positively charged nitrogen atom,
and t, R^{aa}, R^{xy}, R^{yy}, M and B are as defined in claim 22.

24. The compound according to claim 22, wherein R² is selected from the group consisting of 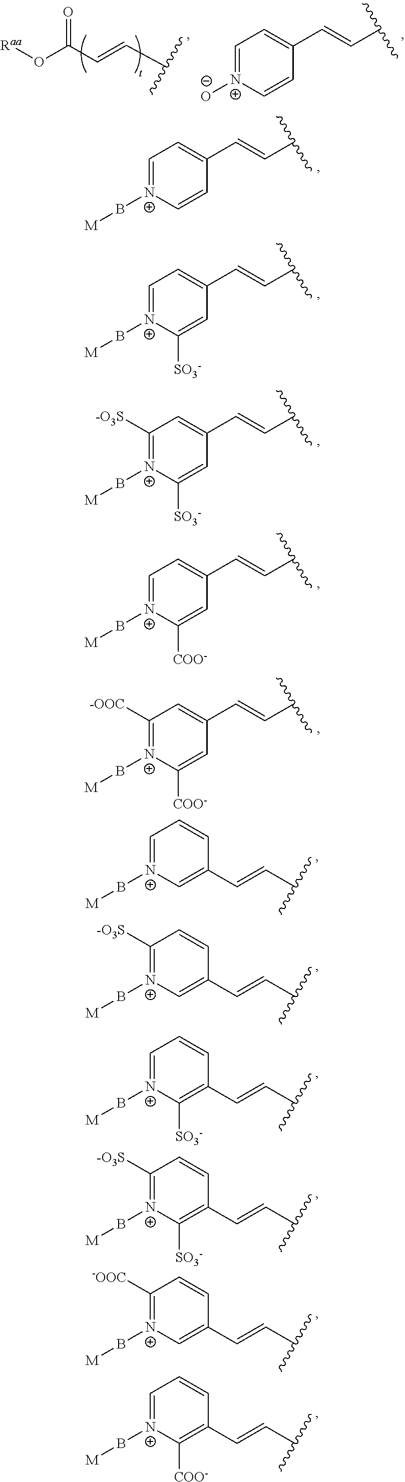 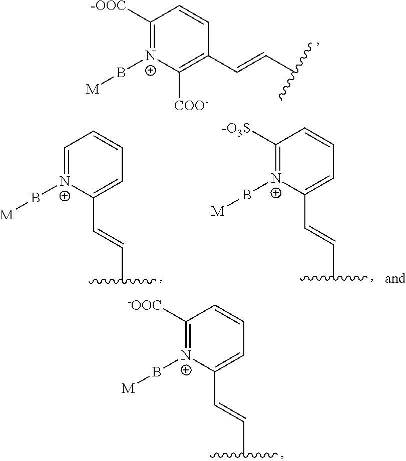
wherein t, R^{aa}, M and B are as defined above, preferably R² is

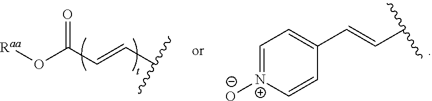

25. The compound according to claim 24, wherein R² is selected from the group consisting of 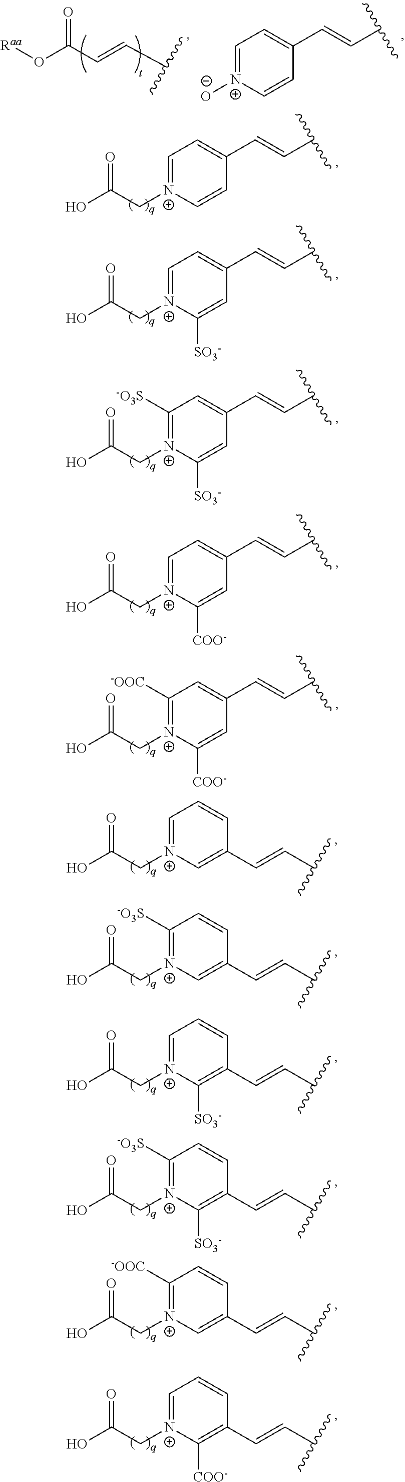 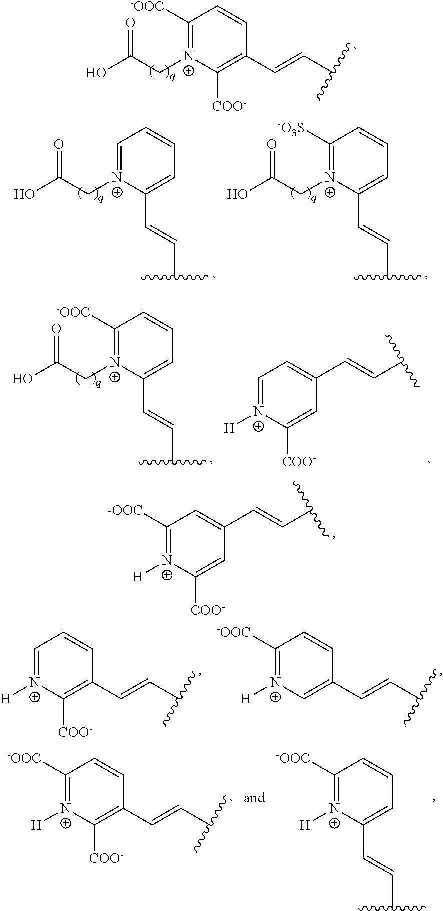
wherein t and R^{aa} are as defined in claim 22, q is 2, 3, 4, 5, 6, 7, or 8, preferably 3, 4, 5, 6, or 7, more preferably 5,
preferably R² is

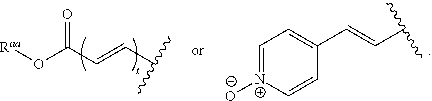

26. The compound according to claim 22, wherein R¹ is selected from the group consisting of
sulfate, i.e. 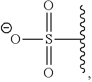 [SMILES: *S(=O)(=O)[O-]]
wherein preferably X is —O— if L is present, and Y is —O— if L is absent;
pyrophosphate diester disodium salt, i.e. 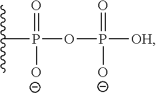 [SMILES: *P(=O)([O-])OP(=O)([O-])O]
wherein preferably X is —O— if L is present, and Y is —O— if L is absent;
phosphoethanolamine, i.e. 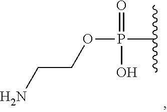 [SMILES: *P(=O)(O)OCCN]
wherein preferably X is —O— if L is present, and Y is —O— if L is absent;
elaidate, i.e. 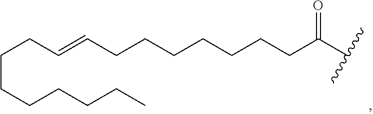 [SMILES: *C(=O)CCCCCCC/C=C/CCCCCCCC]
wherein preferably X is —O— if L is present, and Y is —O— if L is absent;
oleate, i.e. 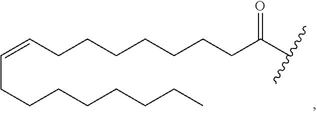 [SMILES: *C(=O)CCCCCCC/C=C\CCCCCCCC]
wherein preferably X is —O— if L is present, and Y is —O— if L is absent;
methyl ether;
ethyl ether;
benzyl ether;
2-deoxy-2-sulfamino-beta-D-glucopyranoside, i.e., 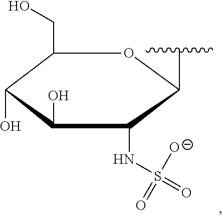
wherein preferably X is —O— if L is present, and Y is —O— if L is absent;
beta-D-glucoside-6-phosphoethanolamine, i.e. 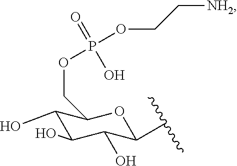 [SMILES: *C1OC(COP(=O)(O)OCCN)C(O)C(O)C1O]
wherein preferably X is —O— if L is present, and Y is —O— if L is absent;
N-acetyl-beta-D-glucosamine, i.e. 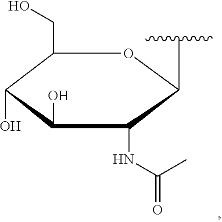
wherein preferably X is —O— if L is present, and Y is —O— if L is absent;
2-acetamido-2-deoxy-b-D-glucopyranoside-6-phosphocholine, i.e., 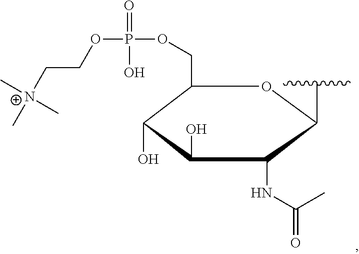
wherein preferably X is —O— if L is present, and Y is —O— if L is absent;
2-acetamido-2-deoxy-alpha-D-glucopyranoside-6-sulfate, i.e. 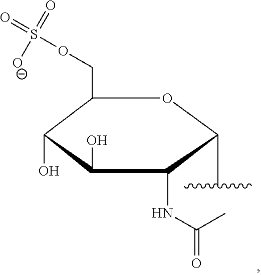
wherein preferably X is —O— if L is present, and Y is —O— if L is absent;
2-acetamido-2-deoxy-4-O-(alpha-L-fucopyranosyl)-beta-D-glucopyranoside, i.e. 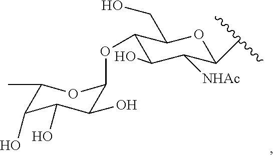 [SMILES: *C1OC(CO)C(OC2OC(C)C(O)C(O)C2O)C(O)C1NC(C)=O]
wherein preferably X is —O— if L is present, and Y is —O— if L is absent;
(6-thio-palmitoy1)-beta-D-glucopyranoside, i.e., 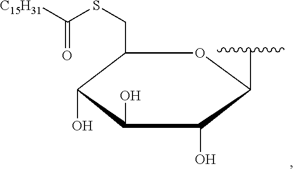
wherein preferably X is —O— if L is present, and Y is —O— if L is absent;
beta-D-lactoside, i.e. 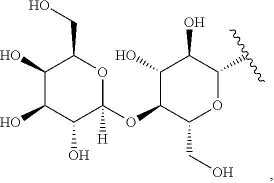 [SMILES: *[C@@H]1O[C@H](CO)[C@@H](O[C@@H]2O[C@H](CO)[C@H](O)[C@H](O)[C@H]2O)[C@H](O)[C@H]1O]
wherein preferably X is —O— if L is present, and Y is —O— if L is absent;
beta-D-galactopyranoside-6-sulfate, i.e. 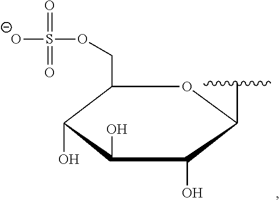
wherein preferably X is —O— if L is present, and Y is —O— if L is absent;
3-O-(alpha-L-fucopyranosyl)-beta-D-galactopyranoside, i.e. 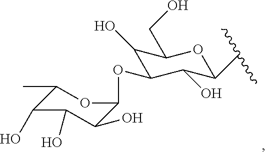 [SMILES: *C1OC(CO)C(O)C(OC2OC(C)C(O)C(O)C2O)C1O]
wherein preferably X is —O— if L is present, and Y is —O— if L is absent;
4-O-(alpha-L-fucopyranosyl)-beta-D-galactopyranoside, i.e. 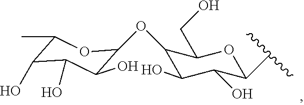 [SMILES: *C1OC(CO)C(OC2OC(C)C(O)C(O)C2O)C(O)C1O]
wherein preferably X is —O— if L is present, and Y is —O— if L is absent;
2-acetamido-2-deoxy-3,6-di-O-pivaloyl-beta-D-galactopyranoside, i.e. 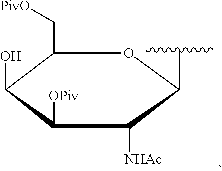
wherein preferably X is —O— if L is present, and Y is —O— if L is absent;
2-acetamido-2-deoxy-beta-D-galactopyranoside-4-sulfate, i.e. 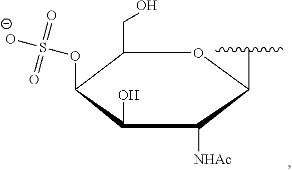
wherein preferably X is —O— if L is present, and Y is —O— if L is absent;
alpha-D-mannopyranoside, i.e. 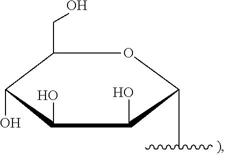
wherein preferably X is —O— if L is present, and Y is —O— if L is absent;
beta-D-mannopyranoside, i.e. 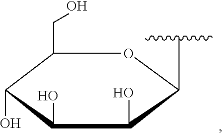
wherein preferably X is —O— if L is present, and Y is —O— if L is absent;
alpha-D-mannopyranoside 6-sulfate, i.e. 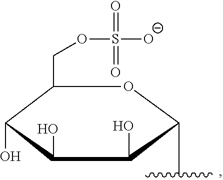
wherein preferably X is —O— if L is present, and Y is —O— if L is absent;
alpha-D-mannopyranoside-2-phosphoethanolamine, i.e. 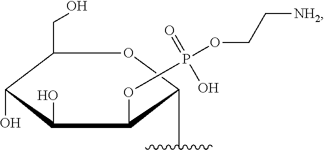
wherein preferably X is —O— if L is present, and Y is —O— if L is absent;
alpha-D-mannopyranoside-6-phosphoethanolamine, i.e. 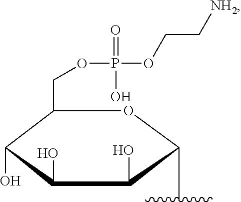
wherein preferably X is —O— if L is present, and Y is —O— if L is absent;
alpha-L-idopyranoside, i.e. 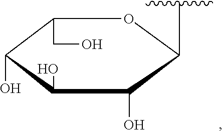
wherein preferably X is —O— if L is present, and Y is —O— if L is absent;
alpha-L-idopyranosiduronic acid, i.e. 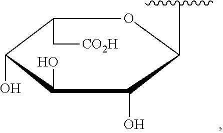
wherein preferably X is —O— if L is present, and Y is —O— if L is absent;
alpha-L-idopyranosiduronic acid 2-sulphate disodium salt, i.e. 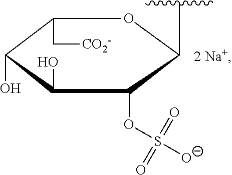
wherein preferably X is —O— if L is present, and Y —O— if L is absent;
alpha-L-rhamnopyranoside, i.e. 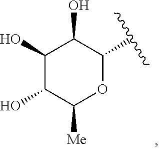 [SMILES: *[C@@H]1O[C@@H](C)[C@H](O)[C@@H](O)[C@H]1O]
wherein preferably X is —O— if L is present, and Y is —O— if L is absent;
a-D-N-glycolylneuraminic acid, i.e. 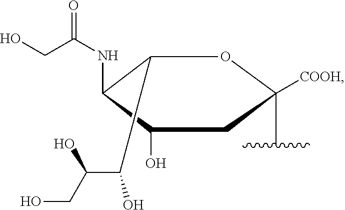
wherein preferably X is —O— if L is present, and Y is —O— if L is absent.
3-deoxy-D-glycero-a-D-galacto-2-nonulosonic acid, i.e. 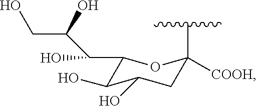 [SMILES: *C1(C(=O)O)CC(O)C(O)C([C@H](O)[C@H](O)CO)O1]
wherein preferably X is —O— if L is present, and Y is —O— if L is absent;
a-L-fucopyranoside, i.e. 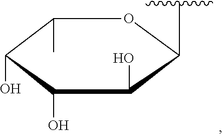
wherein preferably X is —O— if L is present, and Y is —O— if L is absent;
b-L-fucopyranoside, i.e. 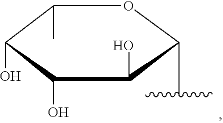
wherein preferably X is —O— if L is present, and Y is —O— if L is absent;
b-D-fucopyranoside, i.e. 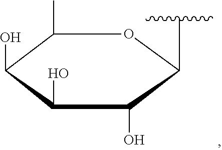
wherein preferably X is —O— if L is present, and Y is —O— if L is absent;
a-L-arabinofuranoside, i.e. 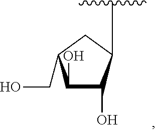
wherein preferably X is —O— if L is present, and Y is —O— if L is absent;
a-L-arabinopyranoside, i.e. 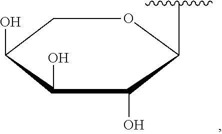
wherein preferably X is —O— if L is present, and Y is —O— if L is absent;
a-D-ribofuranoside, i.e. 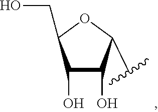
wherein preferably X is —O— if L is present, and Y is —O— if L is absent;
b-D-ribofuranoside, i.e. 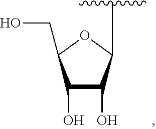
wherein preferably X is —O— if L is present, and Y is —O— if L is absent;
a-D-xylopyranoside, i.e. 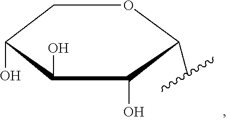
wherein preferably X is —O— if L is present, and Y is —O— if L is absent;
b-D-xylopyranoside, i.e. 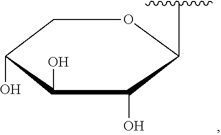
wherein preferably X is —O— if L is present, and Y is —O— if L is absent;
b-D-chitobioside, i.e. 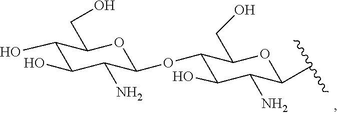 [SMILES: *C1OC(CO)C(OC2OC(CO)C(O)C(O)C2N)C(O)C1N]
wherein preferably X is —O— if L is present, and Y is —O— if L is absent;
4-deoxy-b-D-chitobioside, i.e. 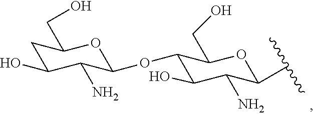 [SMILES: *C1OC(CO)C(OC2OC(CO)CC(O)C2N)C(O)C1N]
wherein preferably X is —O— if L is present, and Y is —O— if L is absent;
N,N-diacetyl-b-D-chitobioside, i.e. 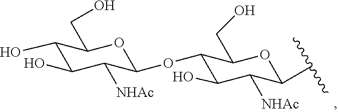 [SMILES: *C1OC(CO)C(OC2OC(CO)C(O)C(O)C2NC(C)=O)C(O)C1NC(C)=O]
wherein preferably X is —O— if L is present, and Y is —O— if L is absent;
N,N′,N″-triacetyl-b-D-chitotrioside, i.e. 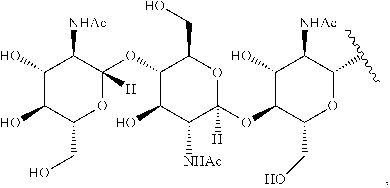 [SMILES: *[C@@H]1O[C@H](CO)[C@@H](O[C@@H]2O[C@H](CO)[C@@H](O[C@@H]3O[C@H](CO)[C@@H](O)[C@H](O)[C@H]3NC(C)=O)[C@H](O)[C@H]2NC(C)=O)[C@H](O)[C@H]1NC(C)=O]
wherein preferably X is —O— if L is present, and Y is —O— if L is absent;
N,N′,N″,N′′′-tetraacetyl-b-D-chitotetraoside, i.e. 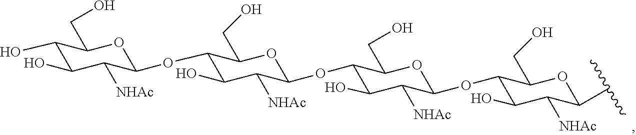 [SMILES: *C1OC(CO)C(OC2OC(CO)C(OC3OC(CO)C(OC4OC(CO)C(O)C(O)C4NC(C)=O)C(O)C3NC(C)=O)C(O)C2NC(C)=O)C(O)C1NC(C)=O]
wherein preferably X is —O— if L is present, and Y is —O— if L is absent;
b-D-cellotrioside, i.e. 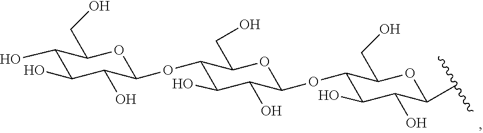 [SMILES: *C1OC(CO)C(OC2OC(CO)C(OC3OC(CO)C(O)C(O)C3O)C(O)C2O)C(O)C1O]
wherein preferably X is —O— if L is present, and Y is —O— if L is absent;
b-D-cellotetraoside, i.e. 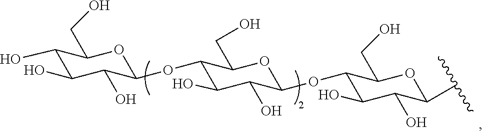
wherein preferably X is —O— if L is present, and Y is —O— if L is absent;
b-D-cellopentoside, i.e. 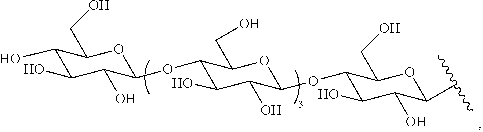
wherein preferably X is —O— if L is present, and Y is —O— if L is absent;
b-D-cellohexaoside, i.e. 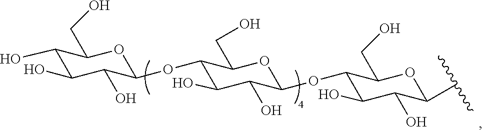
wherein preferably X is —O— if L is present, and Y is —O— if L is absent;
b-D-celloheptaoside, i.e. 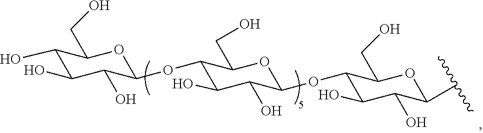
wherein preferably X is —O— if L is present, and Y is —O— if L is absent;
b-D-cellopolyoside, i.e. 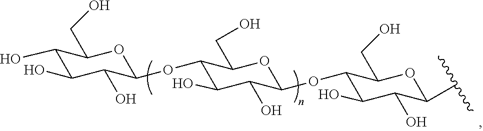
wherein n is 6, 7, 8, 9, 10, 11, 12, 13, 14, 15, or 16, and wherein preferably X
is —O— if L is present, and Y is —O— if L is absent;
b-D-gentiobioside, i.e. 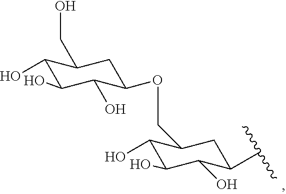 [SMILES: *C1CC(COC2CC(CO)C(O)C(O)C2O)C(O)C(O)C1O]
wherein preferably X is —O— if L is present, and Y is —O— if L is absent;
b-D-gentiotrioside, i.e. 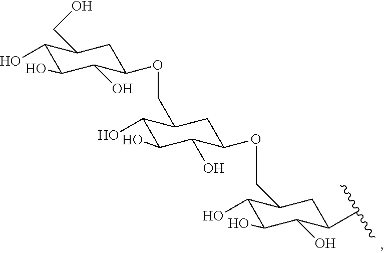 [SMILES: *C1CC(COC2CC(COC3CC(CO)C(O)C(O)C3O)C(O)C(O)C2O)C(O)C(O)C1O]
wherein preferably X is —O— if L is present, and Y is —O— if L is absent;
Maltobioside, i.e. 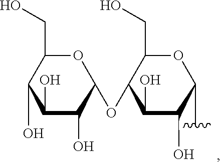
wherein preferably X is —O— if L is present, and Y is —O— if L is absent;
Maltotrioside, i.e. 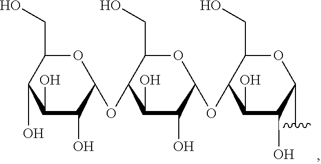
wherein preferably X is —O— if L is present, and Y is —O— if L is absent;
Maltotetraoside, i.e. 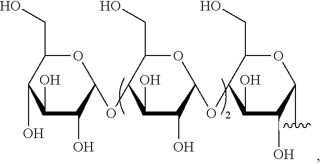
wherein preferably X is —O— if L is present, and Y is —O— if L is absent;
Maltopentaoside, i.e. 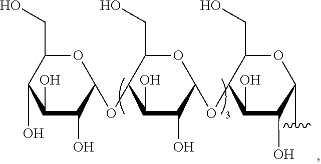
wherein preferably X is —O— if L is present, and Y is —O— if L is absent;
Maltohexaoside, i.e. 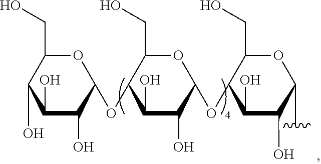
wherein preferably X is —O— if L is present, and Y is —O— if L is absent;
Maltoheptaoside, i.e. 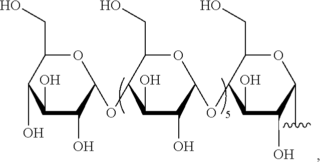
wherein preferably X is —O— if L is present, and Y is —O— if L is absent;
Maltopolyoside, i.e. 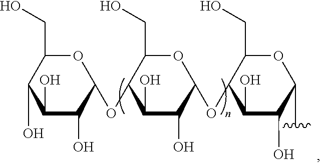
wherein n is 6, 7, 8, 9, 10, 11, 12, 13, 14, 15, or 16, and wherein preferably X
is —O— if L is present, and Y is —O— if L is absent;
b-D-xylobioside, i.e. 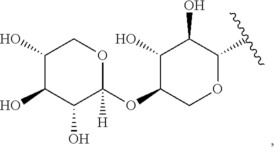 [SMILES: *[C@@H]1OC[C@@H](O[C@@H]2OC[C@@H](O)[C@H](O)[C@H]2O)[C@H](O)[C@H]1O]
wherein preferably X is —O— if L is present, and Y is —O— if L is absent;
b-D-xylotrioside, i.e. 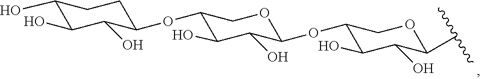 [SMILES: *C1OCC(OC2OCC(OC3CCC(O)C(O)C3O)C(O)C2O)C(O)C1O]
wherein preferably X is —O— if L is present, and Y is —O— if L is absent; 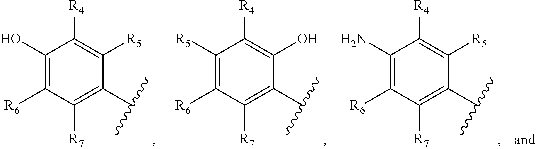 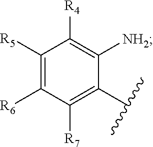
—B(Z)(Z′), —B(Z″)₃⁻ Kat⁺;
—NO₂; 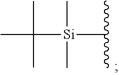 [SMILES: *[Si](C)(C)C(C)(C)C] 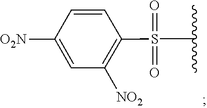 [SMILES: *S(=O)(=O)c1ccc([N+](=O)[O-])cc1[N+](=O)[O-]] 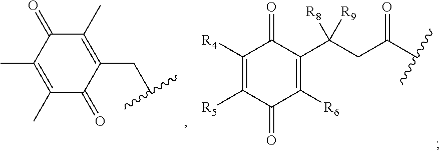 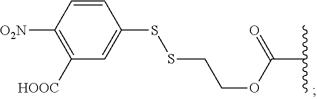 [SMILES: *C(=O)OCCSSc1ccc([N+](=O)[O-])c(C(=O)O)c1]
azide; 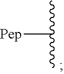
a group having the formula 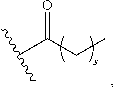
wherein s is 0 or an integer of from 1 to 18, preferably s is 0, 2, 6, 7, and wherein
preferably X is —O— if L is present, and Y is —O— if L is absent;
a group having the formula 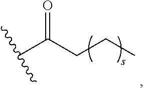
wherein s is 0 or an integer of from 1 to 18, preferably s is 1, and wherein preferably
X is —NH— if L is present;
myo-inositol phosphoryl, wherein preferably X is —O— if L is present, and Y is
—O— if L is absent;
Phosphoryl, wherein preferably X is —O— if L is present, and Y is —O— if L is
absent;
amino acidyl having the formula 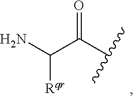
wherein R^{qr} is a side group depending on the respective amino acid,
wherein said amino acidyl is preferably selected from L-alaninyl,
L-leucinyl, and β-alanyl, and wherein X is preferably —NH— if L is present;
di-peptidyl having the formula 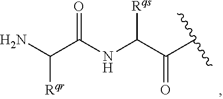
wherein R^{qr} and R^{qs} are side groups depending on the respective
amino acids of which the di-peptidyl group is composed of,
wherein X is preferably —NH— if L is present;
tri-peptidyl having the formula 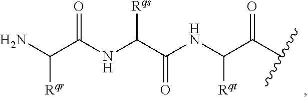
wherein R^{qr}, R^{qs}, and R^{qt} are side groups depending on the respective
amino acids of which the tri-peptidyl group is composed of,
wherein X is preferably —NH— if L is present;
L-pyroglutamic acidyl, i.e. 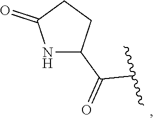 [SMILES: *C(=O)C1CCC(=O)N1]
wherein preferably X is —NH— if L is present;
glycosidyl;
di-saccharidyl;
an amino sugar moiety;
beta-D-galactopyranoside, i.e. 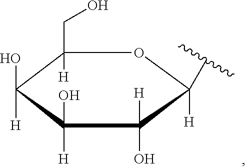
wherein preferably X is —O— if L is present, and Y is —O— if L is absent;
alpha-D-galactopyranoside, i.e. 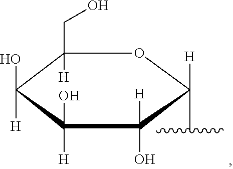
wherein preferably X is —O— if L is present, and Y is —O— if L is absent;
alpha-D-glucopyranoside, i.e. 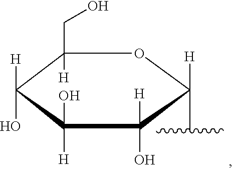
wherein preferably X is —O— if L is present, and Y is —O— if L is absent;
beta-D-glucopyranoside, i.e. 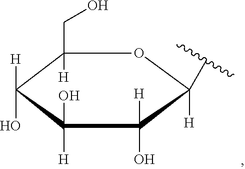
wherein preferably X is —O— if L is present, and Y is —O— if L is absent;
beta-D-glucuronyl, i.e. 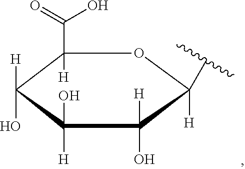
wherein preferably X is —O— if L is present, and Y is —O— if L is absent;
beta-D-glucuronyl sodium salt, i.e. 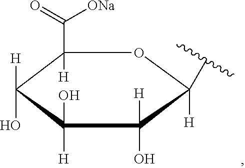
wherein preferably X is —O— if L is present, and Y is —O— if L is absent;
n-acetyl-beta-D-galactosaminidyl, i.e. 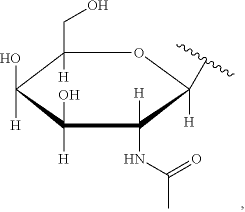
wherein preferably X is —O— if L is present, and Y is —O— if L is absent;
N-acetylneuraminidyl, i.e. 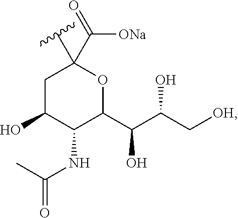
wherein preferably X is —O— if L is present, and Y is —O— if L is absent;
cellobioside, i.e. 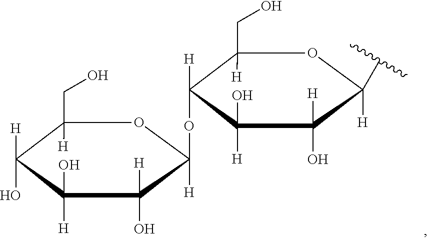
wherein preferably X is —O— if L is present;
choline phosphoryl, phosphoryl, i.e. 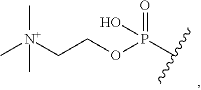 [SMILES: *P(=O)(O)OCC[N+](C)(C)C]
wherein preferably X is —O— if L is present;
oxalylester having the formula 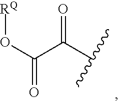
wherein R^{Q} is an optionally substituted C₁C₁₂ alkyl group, wherein X is preferably
—NH— if L is present;
Boc-Val-Pro-Argininyl;
Boc-Asp(OBzl)-Pro-Argininyl;
SucOMe-Arg-Pro-Tyrosinyl (SucOMe-RPY-);
a beta-lactamase-labile group, preferably a beta-lactam antibiotic, more
preferably a penicillin, a cephalosporin of generation 1 to 5, a cephamycin, or a
carbapenem;
Ac-QLQ-;
Ac-FQLQ-;
Ac-EFQLQ-;
Ac-DEFQLQ-;
amides of 5-substituted-o-antranilic acid methyl ester, wherein preferably X is
absent if L is present;
acrylic acid ester, wherein preferably X is —O— if L is present;
L-alanyl (A-);
L-leucinyl (L-);
β-alanyl;
wherein “Pep” is a group comprising a peptide moiety consisting of at least two amino acid residues and linked to L via a carboxylic acid group of said peptide moiety;
provided that when R¹ is 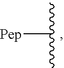
then L is present and X is —NH— or —NR^{G}—, preferably —NH—;
R₄, R₅, R₆, and R₇ are independently selected from the group consisting of hydrogen; C1-C6 alkyl, preferably methyl; halogen, preferably fluorine and chlorine; alkoxy, preferably methoxy; and cyano;
R₈ and R₉ are independently selected from the group consisting of C1-C4 alkyl, preferably methyl, and H, wherein R₈ and R₉ are preferably both methyl.

27. The compound according to claim 22, wherein the compound of Formula Ia has the structure 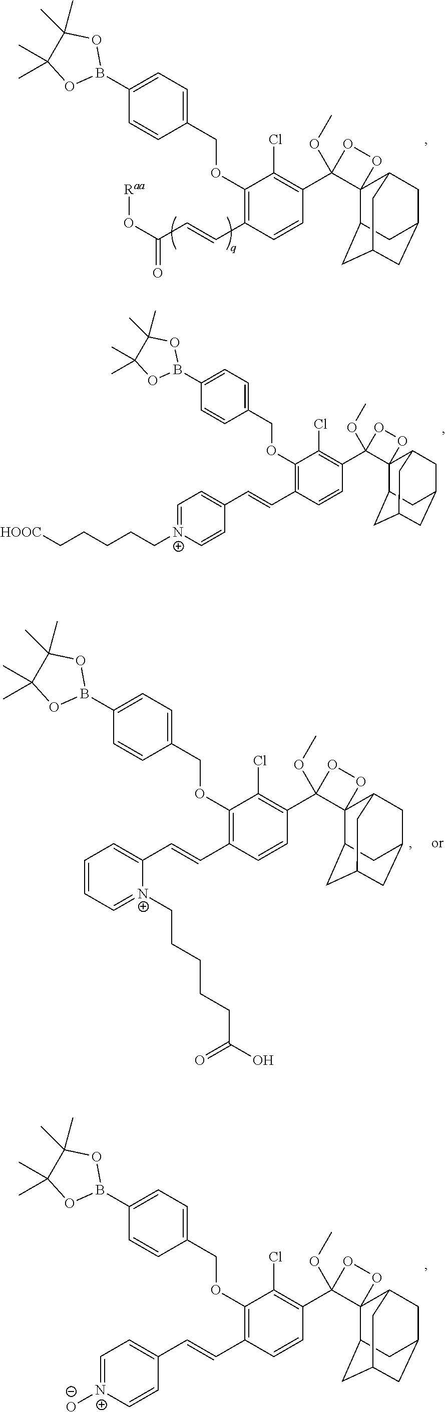
and the compound of Formula Ib has the structure

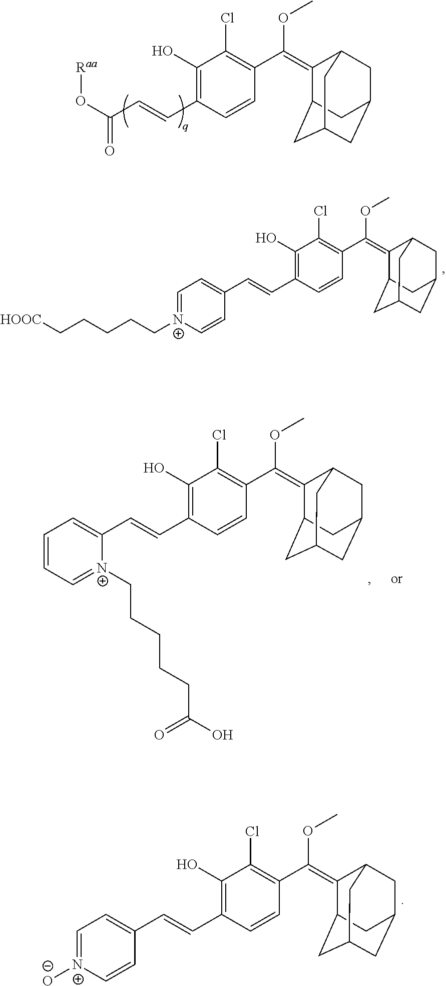

28. A compound of Formula II 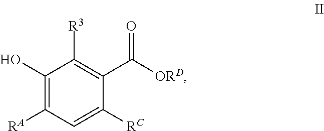
wherein R³, R^{A}, R^{C}, and R^{D} are as defined in claim 22.

29. A composition comprising a compound according to claim 22 and a carrier.

30. A ready-to-use injectable solution comprising a compound according to claim 22.

31. A method for determining the presence, or measuring the level, of an analyte in a sample, the method comprising the following steps:
(a) contacting the sample with a compound according to claim 22, wherein upon exposure to said analyte, said compound reacts with said analyte thereby converting said compound into an emissive species; and
(b) detecting the emission of said emissive species.

32. A biomolecule characterized in that it is bound to a compound of claim 22 as a label.

33. The biomolecule of claim 32, selected from the group consisting of an antibody, a nucleic acid, and a protein.

34. The method of claim 31, wherein the analyte is singlet oxygen, and the compound is a compound of the Formula Ib.

35. The method of claim 31, wherein the sample is a biological sample.

36. The method of claim 35, wherein the biological sample is a bodily fluid, a bodily-based solution, or a tissue biopsy sample.

37. The method of claim 31, wherein the method is an in vitro method.
